(19) United States
(12) Patent Application Publication
(10) Pub. No.: US 2013/0150426 A1
(43) Pub. Date: Jun. 13, 2013

(54) METHODS OF DIAGNOSING AND TREATING IDIOPATHIC PULMONARY FIBROSIS

(71) Applicant: INTERMUNE, INC., BRISBANE, CA (US)

(72) Inventors: KARL KOSSEN, BRISBANE, CA (US); SHARLENE LIM, SAN MATEO, CA (US); XIAOLI QIN, SAN MATEO, CA (US); WILLIAMSON ZIEGLER BRADFORD, ROSS, CA (US); SCOTT D. SEIWERT, PACIFICA, CA (US)

(73) Assignee: INTERMUNE, INC., BRISBANE, CA (US)

(21) Appl. No.: 13/683,099

(22) Filed: Nov. 21, 2012

Related U.S. Application Data
(60) Provisional application No. 61/562,770, filed on Nov. 22, 2011.
(60) Provisional application No. 61/648,548, filed on May 17, 2012.

(51) Int. Cl. C12Q 1/68

(52) U.S. Cl. CPC C12Q 1/6876
USPC 514/44 A; 506/9; 536/24.31; 536/24.33; 514/345

(57) ABSTRACT

Described herein are materials and methods for the diagnosis of idiopathic pulmonary fibrosis.

### CROSS-REFERENCE TO RELATED APPLICATIONS

The present application claims the benefit of priority to U.S. Provisional Application No. 61/562,770, filed Nov. 22, 2011, and U.S. Provisional Application No. 61/648,548, filed May 17, 2012. The disclosure of each priority application is incorporated herein by reference in its entirety.

### FIELD OF THE INVENTION

The present application is directed to methods of diagnosing and treating idiopathic pulmonary fibrosis.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: ASCII (text) file named “46562B_SeqListing.txt,” 37,589 bytes, created on Nov. 20, 2012.

### BACKGROUND

Idiopathic pulmonary fibrosis (IPF), a chronic interstitial lung disease characterized by the unregulated deposition of extracellular matrix leading to unremitting destruction of normal lung. Patients diagnosed with IPF typically experience progressive pulmonary insufficiency, and most die of respiratory failure. The estimated median survival upon diagnosis is approximately 3 years (ATS/ERS. Am J Respir Crit. Care Med 2002:165(2): 277-304). The ratio of the estimated prevalence (90,000 individuals) and incidence (30,000 individuals) of IPF in the United States reflects this poor prognosis (Raghu G, Weycker D, Edelsberg J, Bradford W Z, Oster G. Incidence and prevalence of idiopathic pulmonary fibrosis. Am J Respir Crit. Care Med 2006:174(7): 810-816).

Idiopathic pulmonary fibrosis (IPF) is the most common form of idiopathic interstitial pneumonia and is characterized by the UIP pattern on histology. IPF has an insidious onset, but once symptoms appear, there is a relentless deterioration of pulmonary function and death within 3-5 years after diagnosis.

miRNAs are a class of small non-coding RNAs of about 19-25 nucleotides that function as post-transcriptional gene regulators; and can regulate the entire set of genes (Lim, et al. Nature 2005. 433:769-73). miRNAs provide important regulatory functions in a variety of biological processes, including development, cell proliferation, differentiation, and apoptosis.

### SUMMARY OF THE INVENTION

In one aspect, described herein is a method of diagnosing idiopathic pulmonary fibrosis (IPF) in a human subject comprising detecting in a blood sample from the subject the level of one, two, three, four, five, six, seven or more microRNAs, wherein a differential expression level (increased or decreased) of the one or more microRNA relative to a predetermined criterion or range is indicative of a diagnosis of IPF. For example, the level of the microRNA may be increased or decreased relative to the level in samples of patients without IPF. The method optionally further comprises the step of comparing the level of the microRNA (preferably a normalized level of microRNA) to a predetermined criterion or range. In related aspects, described herein is a method of diagnosing IPF in a human subject comprising detecting in a blood sample from the subject the level of one, two, three, four, five, six, seven or more microRNAs, wherein detection of a level within a predetermined range correlated to IPF is indicative of a diagnosis of IPF. Alternatively, the detection of a level outside of a predetermined range, correlated to patients without IPF, is indicative of a diagnosis of IPF.

In another aspect, described herein is a method of treating a human subject diagnosed with idiopathic pulmonary fibrosis (IPF) according to any of the diagnostic methods described herein comprising administering a therapeutic agent to the subject to treat IPF.

In yet another aspect, described herein is a method of treating a human subject identified as having idiopathic pulmonary fibrosis (IPF) or at risk of IPF based on an abnormal level of one or more IPF-associated microRNAs in a blood sample of the subject, comprising administering a therapeutic agent to the subject to treat IPF.

In any of the embodiments described herein, the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1π (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g# (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d# (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a# (SEQ ID NO:155), miR-106a (SEQ ID NO:156), miR-1227 (SEQ ID NO: 157), miR-128 (SEQ ID NO:158), miR-132 (SEQ ID NO:159), miR-140-5p (SEQ ID NO: 160), miR-141 (SEQ ID NO:161), miR-17 (SEQ ID NO:162), miR-185 (SEQ ID NO:163), miR-30a-5p (SEQ ID NO:164), miR-30d (SEQ ID NO:165), miR-34a (SEQ ID NO:166), miR-378 (SEQ ID NO:167), miR-425 (SEQ ID NO:168), miR-429 (SEQ ID NO:169), miR-579 (SEQ ID NO:170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO:179), miR-27b# (SEQ ID NO:180), miR-331-3p (SEQ ID NO: 181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO: 193), miR-668 (SEQ ID NO:194), miR-1256 (SEQ ID NO:195), miR-299-5p (SEQ ID NO:196), miR-1183 (SEQ ID NO:197), miR-1233 (SEQ ID NO:198), miR-1247 (SEQ ID NO:199), miR-1249 (SEQ ID NO:200), miR-1270 (SEQ ID NO:201), miR-1274A (SEQ ID NO:202), miR-1275 (SEQ ID NO:203), miR-1298 (SEQ ID NO:204), miR-135b (SEQ ID NO:205), miR-138 (SEQ ID NO:206), miR-145 (SEQ ID NO:207), miR-15a (SEQ ID NO:208), miR-181c (SEQ ID NO:209), miR-186 (SEQ ID NO:210), miR-18a# (SEQ ID NO:211), miR-193a-3p (SEQ ID NO:212), miR-199b-5p (SEQ ID NO:213), miR-200a (SEQ ID NO:214), miR-205 (SEQ ID NO:215), miR-20b (SEQ ID NO:216), miR-214 (SEQ ID NO:217), miR-214# (SEQ ID NO:218), miR-218 (SEQ ID NO:219), miR-220b (SEQ ID NO:220), miR-223 (SEQ ID NO:221), miR-23b (SEQ ID NO:222), miR-30a-3p (SEQ ID NO:223), miR-30e-3p (SEQ ID NO:224), miR-326 (SEQ ID NO:225), miR-346 (SEQ ID NO:226), miR-431 (SEQ ID NO:227), miR-450a (SEQ ID NO:228), miR-450b-5p (SEQ ID NO:229), miR-455-5p (SEQ ID NO:230), miR-487a (SEQ ID NO:231), miR-493 (SEQ ID NO:232), miR-494 (SEQ ID NO:233), miR-501-5p (SEQ ID NO:234), miR-505# (SEQ ID NO:235), miR-511 (SEQ ID NO:236), miR-518d-3p (SEQ ID NO:237), miR-518e (SEQ ID NO:238), miR-518f (SEQ ID NO:239), miR-548c-3p (SEQ ID NO: 240), miR-570 (SEQ ID NO:241), miR-571 (SEQ ID NO:242), miR-577 (SEQ ID NO: 243), miR-618 (SEQ ID NO:244), miR-744# (SEQ ID NO:245), miR-769-5p (SEQ ID NO: 246), miR-885-5p (SEQ ID NO:247), miR-892b (SEQ ID NO:248), miR-9# (SEQ ID NO: 249), miR-95 (SEQ ID NO:250) and combinations thereof, and the levels relative to a predetermined criterion or range are increased or decreased as disclosed herein.

In any of the embodiments described herein, the one, two, three, four, five, six, seven or more microRNA are selected from the group consisting of miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-197 (SEQ ID NO:10), miR-663B (SEQ ID NO:11), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), let-7d (SEQ ID NO:45), miR-361-5p (SEQ ID NO:48), miR-520e (SEQ ID NO:49), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-17# (SEQ ID NO:58), miR-32 (SEQ ID NO:60), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144 (SEQ ID NO:69), miR-26a (SEQ ID NO:70), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-20a (SEQ ID NO:89), miR-28-5p (SEQ ID NO:90), miR-148a (SEQ ID NO:91), let-7e (SEQ ID NO:93), miR-656 (SEQ ID NO:95), miR-362-3p (SEQ ID NO:96), miR-423-5p (SEQ ID NO:99), miR-652 (SEQ ID NO:100), miR-127-3p (SEQ ID NO:101), miR-495 (SEQ ID NO:102), miR-590-5p (SEQ ID NO:104), miR-103 (SEQ ID NO:105), miR-324-3p (SEQ ID NO:107), miR-598 (SEQ ID NO:110), miR-130a (SEQ ID NO:112), miR-502-3p (SEQ ID NO:113), miR-194 (SEQ ID NO:115), miR-221 (SEQ ID NO:116), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-411 (SEQ ID NO:124), miR-29b (SEQ ID NO:125), miR-340# (SEQ ID NO:127), miR-148b# (SEQ ID NO:128), miR-212 (SEQ ID NO:129), miR-152 (SEQ ID NO:130), miR-7 (SEQ ID NO:132), miR-543 (SEQ ID NO:133), miR-30d# (SEQ ID NO:134), miR-213 (SEQ ID NO:135), miR-1197 (SEQ ID NO:137), miR-1255B (SEQ ID NO:138), miR-154 (SEQ ID NO:139), miR-196b (SEQ ID NO:140), miR-21# (SEQ ID NO:141), miR-335 (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), let-7a# (SEQ ID NO:155), miR-106a (SEQ ID NO:156), miR-1227 (SEQ ID NO:157), miR-128 (SEQ ID NO:158), miR-132 (SEQ ID NO:159), miR-140-5p (SEQ ID NO:160), miR-141 (SEQ ID NO:161), miR-17 (SEQ ID NO:162), miR-185 (SEQ ID NO:163), miR-30a-5p (SEQ ID NO:164), miR-30d (SEQ ID NO:165), miR-34a (SEQ ID NO:166), miR-378 (SEQ ID NO:167), miR-425 (SEQ ID NO: 168), miR-429 (SEQ ID NO:169), miR-579 (SEQ ID NO:170), miR-523 (SEQ ID NO: 171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO: 177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof, and the levels relative to a predetermined criterion or range are increased or decreased as disclosed herein.

In any of the embodiments described herein, the one, two, three, four, five, six, seven or more microRNA are selected from the group consisting of miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-197 (SEQ ID NO:10), miR-663B (SEQ ID NO:11), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-572 (SEQ ID NO:35), miR-886-3p (SEQ ID NO:36), miR-26b (SEQ ID NO:40), miR-142-5p (SEQ ID NO:43), let-7d (SEQ ID NO:45), miR-361-5p (SEQ ID NO:48), miR-520e (SEQ ID NO:49), miR-148b (SEQ ID NO:53), miR-15b# (SEQ ID NO:55), miR-15b (SEQ ID NO:56), miR-17# (SEQ ID NO:58), miR-190 (SEQ ID NO:63), miR-15a# (SEQ ID NO:64), miR-181a (SEQ ID NO:66), miR-301a (SEQ ID NO:67), miR-374a (SEQ ID NO:68), miR-144# (SEQ ID NO:69), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-28-5p (SEQ ID NO:90), miR-148a (SEQ ID NO:91), let-7e (SEQ ID NO:93), miR-656 (SEQ ID NO:95), miR-362-3p (SEQ ID NO:96), miR-652 (SEQ ID NO:100), miR-127-3p (SEQ ID NO:101), miR-495 (SEQ ID NO:102), miR-590-5p (SEQ ID NO:104), miR-598 (SEQ ID NO:110), miR-130a (SEQ ID NO:112), miR-502-3p (SEQ ID NO:113), miR-194 (SEQ ID NO:115), miR-221 (SEQ ID NO:116), miR-335 (SEQ ID NO:119), miR-24-2# (SEQ ID NO:120), miR-130b (SEQ ID NO:121), miR-99b (SEQ ID NO:122), miR-411 (SEQ ID NO:124), miR-340# (SEQ ID NO:127), miR-148b# (SEQ ID NO:128), miR-212 (SEQ ID NO:129), miR-152 (SEQ ID NO:130), miR-7 (SEQ ID NO:132), miR-543 (SEQ ID NO:133), miR-30d (SEQ ID NO:134), miR-213 (SEQ ID NO:135), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), let-7a# (SEQ ID NO:155), miR-106a (SEQ ID NO:156), miR-1227 (SEQ ID NO: 157), miR-140-5p (SEQ ID NO:160), miR-141 (SEQ ID NO:161), miR-17 (SEQ ID NO: 162), miR-185 (SEQ ID NO:163), miR-30a-5p (SEQ ID NO:164), miR-30d (SEQ ID NO: 165), miR-34a (SEQ ID NO:166), miR-378 (SEQ ID NO:167), miR-425 (SEQ ID NO: 168), miR-429 (SEQ ID NO:169), miR-579 (SEQ ID NO:170), miR-523 (SEQ ID NO: 171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO: 177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof, and the levels relative to a predetermined criterion or range are increased or decreased as disclosed herein.

In any of the embodiments described herein, the one, two, three, four, five, six, seven or more microRNA are selected from the group consisting of let-7a (SEQ ID NO:173), let-7a# (SEQ ID NO:155), let-7d (SEQ ID NO:45), miR-106a (SEQ ID NO:156), let-7e (SEQ ID NO:93), miR-122 (SEQ ID NO:22), let-7f (SEQ ID NO:174), miR-1227 (SEQ ID NO:157), let-7g (SEQ ID NO:72), miR-128 (SEQ ID NO:158), miR-103(SEQ ID NO:105), miR-130a (SEQ ID NO:112), miR-107 (SEQ ID NO:175), miR-132 (SEQ ID NO:159), miR-1244(SEQ ID NO:20), miR-140-5p (SEQ ID NO:160), miR-1256 (SEQ ID NO:195), miR-141 (SEQ ID NO:161), miR-125a-5p (SEQ ID NO:176), miR-142-5p (SEQ ID NO: 43), miR-127-3p (SEQ ID NO:101), miR-146a (SEQ ID NO:21), miR-142-3p (SEQ ID NO:38), miR-146b-5p (SEQ ID NO:24), miR-144# (SEQ ID NO:69), miR-148a (SEQ ID NO:91), miR-148b# (SEQ ID NO:128), miR-150 (SEQ ID NO:27), miR-154# (SEQ ID NO:139), miR-152 (SEQ ID NO:130), miR-15b (SEQ ID NO:56), miR-15a# (SEQ ID NO: 64), miR-181a-2# (SEQ ID NO:177), miR-17 (SEQ ID NO:162), miR-190 (SEQ ID NO: 63), miR-185 (SEQ ID NO:163), miR-196b (SEQ ID NO:140), miR-19a (SEQ ID NO:74), miR-19b-1# (SEQ ID NO:178), miR-21 (SEQ ID NO:51), miR-200c (SEQ ID NO:179), miR-21# (SEQ ID NO:141), miR-20a# (SEQ ID NO:75), miR-222 (SEQ ID NO:16), miR-24-2# (SEQ ID NO:120), miR-26a-2# (SEQ ID NO:82), miR-26a (SEQ ID NO:70), miR-30a-5p (SEQ ID NO:164), miR-27b# (SEQ ID NO:180), miR-30d (SEQ ID NO:165), miR-28-5p (SEQ ID NO:90), miR-324-3p (SEQ ID NO:107), miR-299-5p (SEQ ID NO:196), miR-335 (SEQ ID NO:119), miR-29b (SEQ ID NO:125), miR-345 (SEQ ID NO:18), miR-301a (SEQ ID NO:67), miR-34a (SEQ ID NO:166), miR-30b (SEQ ID NO:42), miR-362-3p (SEQ ID NO:96), miR-30c (SEQ ID NO:52), miR-378 (SEQ ID NO:167), miR-331-3p (SEQ ID NO:181), miR-425 (SEQ ID NO:168), miR-339-5p (SEQ ID NO:182), miR-429 (SEQ ID NO:169), miR-340# (SEQ ID NO:127), miR-523 (SEQ ID NO:171), miR-362-5p (SEQ ID NO:183), miR-551b# (SEQ ID NO:172), miR-370 (SEQ ID NO:184), miR-579 (SEQ ID NO:170), miR-374a (SEQ ID NO:68), miR-590-5p (SEQ ID NO:104), miR-374b (SEQ ID NO:185), miR-598 (SEQ ID NO:110), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO:190), miR-766 (SEQ ID NO:191), miR-9 (SEQ ID NO:192), miR-98 (SEQ ID NO:193), miR-99b (SEQ ID NO:122), and combinations thereof, and the levels relative to a predetermined criterion or range are increased or decreased as disclosed herein.

In any of the embodiments described herein, the one, two, three, four, five, six, seven or more microRNA are selected from the group consisting of let-7a (SEQ ID NO:173), let-7a# (SEQ ID NO:155), miR-106a (SEQ ID NO:156), let-7e (SEQ ID NO:93), miR-122 (SEQ ID NO:22), let-7f (SEQ ID NO:174), miR-1227 (SEQ ID NO:157), let-7g (SEQ ID NO:72), miR-130a (SEQ ID NO:112), miR-107 (SEQ ID NO:175), miR-1244(SEQ ID NO:20), miR-140-5p (SEQ ID NO:160), miR-1256 (SEQ ID NO:195), miR-141 (SEQ ID NO:161), miR-125a-5p (SEQ ID NO:176), miR-142-5p (SEQ ID NO:43), miR-127-3p (SEQ ID NO:101), miR-146a (SEQ ID NO:21), miR-146b-5p (SEQ ID NO:24), miR-144 (SEQ ID NO:69), miR-148a (SEQ ID NO:91), miR-148b# (SEQ ID NO:128), miR-154 (SEQ ID NO:139), miR-152 (SEQ ID NO:130), miR-15b (SEQ ID NO:56), miR-15a# (SEQ ID NO:64), miR-181a-2# (SEQ ID NO:177), miR-17 (SEQ ID NO:162), miR-190 (SEQ ID NO:63), miR-185 (SEQ ID NO:163), miR-196b (SEQ ID NO:140), miR-19a (SEQ ID NO:74), miR-19b-1# (SEQ ID NO:178), miR-200c (SEQ ID NO:179), miR-21# (SEQ ID NO:141), miR-20a# (SEQ ID NO:75), miR-222 (SEQ ID NO:16), miR-24-2# (SEQ ID NO:120), miR-26a-2# (SEQ ID NO:82), miR-30a-5p (SEQ ID NO:164), miR-27b# (SEQ ID NO:180), miR-30d (SEQ ID NO:165), miR-28-5p (SEQ ID NO:90), miR-299-5p (SEQ ID NO:196), miR-335 (SEQ ID NO:119), miR-345 (SEQ ID NO:18), miR-301a (SEQ ID NO:67), miR-34a (SEQ ID NO:166), miR-362-3p (SEQ ID NO:96), miR-378 (SEQ ID NO:167), miR-331-3p (SEQ ID NO:181), miR-425 (SEQ ID NO:168), miR-339-5p (SEQ ID NO:182), miR-429 (SEQ ID NO:169), miR-340# (SEQ ID NO:127), miR-523 (SEQ ID NO:171), miR-362-5p (SEQ ID NO:183), miR-551b# (SEQ ID NO:172), miR-370 (SEQ ID NO:184), miR-579 (SEQ ID NO:170), miR-374a (SEQ ID NO:68), miR-590-5p (SEQ ID NO:104), miR-374b (SEQ ID NO:185), miR-598 (SEQ ID NO:110), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO:190), miR-766 (SEQ ID NO:191), miR-9 (SEQ ID NO: 192), miR-98 (SEQ ID NO:193), miR-99b (SEQ ID NO:122), and combinations thereof, and the levels relative to a predetermined criterion or range are increased or decreased as disclosed herein.

In any of the embodiments described herein, the one, two, three, four, five, six, seven or more microRNA are selected from the group consisting of let-7a (SEQ ID NO:173), miR-132 (SEQ ID NO:159), let-7d (SEQ ID NO:45), miR-148a (SEQ ID NO:91), let-7e (SEQ ID NO:93), miR-152 (SEQ ID NO:130), let-7f (SEQ ID NO:174), miR-17 (SEQ ID NO:162), miR-103 (SEQ ID NO:105), miR-185 (SEQ ID NO:163), miR-1256 (SEQ ID NO:195), miR-21 (SEQ ID NO:51), miR-142-3p (SEQ ID NO:38), miR-222 (SEQ ID NO: 16), miR-144# (SEQ ID NO:69), miR-345 (SEQ ID NO:18), miR-148b# (SEQ ID NO: 128), miR-34a (SEQ ID NO:166), miR-154# (SEQ ID NO:139), miR-523 (SEQ ID NO: 171), miR-15b (SEQ ID NO:56), miR-551b# (SEQ ID NO:172), miR-181a-2# (SEQ ID NO:177), miR-590-5p (SEQ ID NO:104), miR-190 (SEQ ID NO:63), miR-20a# (SEQ ID NO:75), miR-24-2# (SEQ ID NO:120), miR-26a (SEQ ID NO:70), miR-28-5p (SEQ ID NO:90), miR-299-5p (SEQ ID NO:196), miR-30b (SEQ ID NO:42), miR-30c (SEQ ID NO:52), miR-331-3p (SEQ ID NO:181), miR-340# (SEQ ID NO:127), miR-362-5p (SEQ ID NO:183), miR-374a (SEQ ID NO:68), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-668 (SEQ ID NO:194), miR-758 (SEQ ID NO:190), and combinations thereof, and the levels relative to a predetermined criterion or range are increased or decreased as disclosed herein.

In any of the embodiments described herein, the one, two, three, four, five, six, seven or more microRNA are selected from the group consisting of let-7a (SEQ ID NO:173), miR-148a (SEQ ID NO:91), let-7e (SEQ ID NO:93), miR-152 (SEQ ID NO:130), let-7f (SEQ ID NO:174), miR-17 (SEQ ID NO:162), miR-185 (SEQ ID NO:163), miR-1256 (SEQ ID NO:195), miR-222 (SEQ ID NO:16), miR-144# (SEQ ID NO:69), miR-345 (SEQ ID NO:18), miR-148b# (SEQ ID NO:128), miR-34a (SEQ ID NO:166), miR-154 (SEQ ID NO:139), miR-523 (SEQ ID NO:171), miR-15b (SEQ ID NO:56), miR-551b# (SEQ ID NO:172), miR-181a-2# (SEQ ID NO:177), miR-590-5p (SEQ ID NO:104), miR-190 (SEQ ID NO:63), miR-20a# (SEQ ID NO:75), miR-24-2# (SEQ ID NO:120), miR-28-5p (SEQ ID NO:90), miR-299-5p (SEQ ID NO:196), miR-331-3p (SEQ ID NO:181), miR-340# (SEQ ID NO:127), miR-362-5p (SEQ ID NO:183), miR-374a (SEQ ID NO:68), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-668 (SEQ ID NO:194), miR-758 (SEQ ID NO:190), and combinations thereof, and the levels relative to a predetermined criterion or range are increased or decreased as disclosed herein.

In any of the embodiments described herein, the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), and combinations thereof, and the levels relative to a predetermined criterion or range are increased or decreased as disclosed herein.

In any of the embodiments described herein, the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), and combinations thereof, and the levels relative to a predetermined criterion or range are increased or decreased as disclosed herein.

In any of the embodiments described herein, the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), and combinations thereof, and the levels relative to a predetermined criterion or range are increased or decreased as disclosed herein.

In any of the embodiments described herein, the one, two, three, four, five, six, seven or more microRNA are selected from the group consisting of let-7a (SEQ ID NO:173), let-7e (SEQ ID NO:93), let-7f (SEQ ID NO:174), let-7g (SEQ ID NO:72), miR-107 (SEQ ID NO:175), miR-1244 (SEQ ID NO:20), miR-1256 (SEQ ID NO:195), miR-127-3p (SEQ ID NO:101), miR-144# (SEQ ID NO:69), miR-148b# (SEQ ID NO:128), miR-154# (SEQ ID NO:139), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-27b# (SEQ ID NO:180), miR-28-5p (SEQ ID NO:90), miR-299-5p (SEQ ID NO:196), miR-301a (SEQ ID NO:67), miR-331-3p (SEQ ID NO:181), miR-339-5p (SEQ ID NO:182), miR-340 (SEQ ID NO:127), miR-362-5p (SEQ ID NO:183), miR-370 (SEQ ID NO:184), miR-374a (SEQ ID NO:68), miR-374b (SEQ ID NO:185), miR-411 (SEQ ID NO:124), miR-411 (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO:190), miR-766 (SEQ ID NO:191), miR-9 (SEQ ID NO:192), miR-98 (SEQ ID NO:193), miR-99b (SEQ ID NO:122), and combinations thereof, and the levels relative to a predetermined criterion or range are increased or decreased as disclosed herein.

In any of the embodiments described herein, the one, two, three, four, five, six, seven or more microRNA are selected from the group consisting of let-7d (SEQ ID NO:45), miR-103 (SEQ ID NO:105), miR-125a-5p (SEQ ID NO:176), miR-142-3p (SEQ ID NO: 38), miR-26a (SEQ ID NO:70), miR-29b (SEQ ID NO:125), miR-30b (SEQ ID NO:42), miR-30c (SEQ ID NO:52), miR-379 (SEQ ID NO:186), and combinations thereof, and the levels relative to a predetermined criterion or range are increased or decreased as disclosed herein.

In any of the embodiments described herein, the one, two, three, four, five, six, seven or more microRNA are selected from the group consisting of miR-18a (SEQ ID NO: 39), miR-26b (SEQ ID NO:40), miR-106b (SEQ ID NO:41), miR-29c (SEQ ID NO:44), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-148b (SEQ ID NO:53), miR-27b (SEQ ID NO:54), miR-15b# (SEQ ID NO:55), miR-16-1# (SEQ ID NO:57), miR-17 (SEQ ID NO:58), miR-22 (SEQ ID NO:59), miR-32 (SEQ ID NO:60), miR-532-5p (SEQ ID NO:61), miR-101 (SEQ ID NO:62), miR-27a (SEQ ID NO:65), miR-181a (SEQ ID NO: 66), miR-320B (SEQ ID NO:71), miR-324-5p (SEQ ID NO:73), let-7b (SEQ ID NO:76), miR-422a (SEQ ID NO:77), let-7f-2# (SEQ ID NO:78), let-7g# (SEQ ID NO:79), miR-128a (SEQ ID NO:80), miR-199a-5p (SEQ ID NO:81), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-106b# (SEQ ID NO:92), miR-25 (SEQ ID NO:94), miR-656 (SEQ ID NO:95), miR-340 (SEQ ID NO:97), miR-451 (SEQ ID NO:98), miR-423-5p (SEQ ID NO:99), miR-652 (SEQ ID NO:100), miR-495 (SEQ ID NO:102), miR-328 (SEQ ID NO:103), miR-19b (SEQ ID NO:106), miR-145# (SEQ ID NO:108), miR-199a-3p (SEQ ID NO:109), miR-151-5P (SEQ ID NO:111), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-130b (SEQ ID NO:121), miR-195 (SEQ ID NO:123), miR-576-3p (SEQ ID NO:126), miR-212 (SEQ ID NO:129), miR-143 (SEQ ID NO:131), dme-miR-7 (SEQ ID NO:132), miR-30d# (SEQ ID NO:134), miR-213 (SEQ ID NO:135), miR-126# (SEQ ID NO:136), miR-1197 (SEQ ID NO:137), miR-1255B (SEQ ID NO:138), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-551b (SEQ ID NO:149), miR-616 (SEQ ID NO:150), miR-638 (SEQ ID NO:151), miR-889 (SEQ ID NO:153), rno-miR-29c# (SEQ ID NO:154), and combinations thereof, and the levels relative to a predetermined criterion or range are increased or decreased as disclosed herein.

In any of the embodiments described herein, the one, two, three, four, five, six, seven or more microRNA are selected from the group consisting of let-7a (SEQ ID NO:173), let-7d (SEQ ID NO:45), let-7e (SEQ ID NO:93), let-7f (SEQ ID NO:174), miR-103 (SEQ ID NO:105), miR-142-3p (SEQ ID NO:38), miR-144# (SEQ ID NO:69), miR-148b# (SEQ ID NO:128), miR-154# (SEQ ID NO:139), miR-15b (SEQ ID NO:56), miR-181a-2# (SEQ ID NO:177), miR-190 (SEQ ID NO:63), miR-20a# (SEQ ID NO:75), miR-24-2# (SEQ ID NO:120), miR-26a (SEQ ID NO:70), miR-28-5p (SEQ ID NO:90), miR-30b (SEQ ID NO: 42), miR-30c (SEQ ID NO:52), miR-331-3p (SEQ ID NO:181), miR-340# (SEQ ID NO: 127), miR-362-5p (SEQ ID NO:183), miR-374a (SEQ ID NO:68), miR-379 (SEQ ID NO: 186), miR-411 (SEQ ID NO:124), miR-411# (SEQ ID NO:147), miR-454 (SEQ ID NO: 187), miR-520a-3p (SEQ ID NO:188), miR-758 (SEQ ID NO:190), miR-1256 (SEQ ID NO:195), miR-299-5p (SEQ ID NO:196), miR-668 (SEQ ID NO:194), and combinations thereof, and the levels relative to a predetermined criterion or range are increased or decreased as disclosed herein.

In any of the embodiments described herein, the one, two, three, four, five, six, seven or more microRNA are selected from the group consisting of let-7g (SEQ ID NO:72), miR-107 (SEQ ID NO:175), miR-1244 (SEQ ID NO:20), miR-125a-5p (SEQ ID NO:176), miR-127-3p (SEQ ID NO:101), miR-196b (SEQ ID NO:140), miR-19b-1# (SEQ ID NO: 178), miR-200c (SEQ ID NO:179), miR-27b# (SEQ ID NO:180), miR-29b (SEQ ID NO: 125), miR-301a (SEQ ID NO:67), miR-339-5p (SEQ ID NO:182), miR-370 (SEQ ID NO: 184), miR-374b (SEQ ID NO:185), miR-539 (SEQ ID NO:189), miR-543 (SEQ ID NO: 133), miR-548J (SEQ ID NO:148), miR-664 (SEQ ID NO:152), miR-766 (SEQ ID NO: 191), miR-9 (SEQ ID NO:192), miR-98 (SEQ ID NO:193), miR-99b (SEQ ID NO:122), and combinations thereof, and the levels relative to a predetermined criterion or range are increased or decreased as disclosed herein.

In any of the embodiments described herein, the one, two, three, four, five, six, seven or more microRNA are selected from the group consisting of let-7a (SEQ ID NO:173), let-7d (SEQ ID NO:45), let-7e (SEQ ID NO:93), let-7f (SEQ ID NO:174), let-7g (SEQ ID NO:72); miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-379 (SEQ ID NO:186), miR-411 (SEQ ID NO:124), miR-454 (SEQ ID NO:187), miR-539 (SEQ ID NO:189), miR-543 (SEQ ID NO:133), miR-548J (SEQ ID NO:148), miR-664 (SEQ ID NO:152), miR-758 (SEQ ID NO:190), miR-766 (SEQ ID NO:191), miR-9 (SEQ ID NO:192), miR-98 (SEQ ID NO:193), miR-99b (SEQ ID NO:122); miR-668 (SEQ ID NO:194), miR-106a (SEQ ID NO:156), miR-122 (SEQ ID NO:22), miR-1227 (SEQ ID NO:157), miR-132 (SEQ ID NO:159), miR-146a (SEQ ID NO:21), miR-17 (SEQ ID NO:162), miR-222 (SEQ ID NO:16), miR-26a-2# (SEQ ID NO: 82), miR-345 (SEQ ID NO:18), miR-34a (SEQ ID NO:166), miR-429 (SEQ ID NO:169), miR-590-5p (SEQ ID NO:104), miR-598 (SEQ ID NO:110), miR-551b# (SEQ ID NO: 172), miR-1183 (SEQ ID NO:197) and miR-892b, (SEQ ID NO:248) and combinations thereof, and the levels relative to a predetermined criterion or range are increased or decreased as disclosed herein.

In any of the embodiments described herein, the one, two, three, four, five, six, seven or more microRNA are selected from the group consisting of let-7a (SEQ ID NO:173), let-7d (SEQ ID NO:45), let-7e (SEQ ID NO:93), let-7f (SEQ ID NO:174), miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-20a# (SEQ ID NO:75), miR-24-2# (SEQ ID NO:120), miR-26a (SEQ ID NO:70), miR-28-5p (SEQ ID NO:90), miR-30b (SEQ ID NO:42), miR-30c (SEQ ID NO:52), miR-331-3p (SEQ ID NO: 181), miR-339-5p (SEQ ID NO:182), miR-340# (SEQ ID NO:127), miR-362-5p (SEQ ID NO:183), miR-370 (SEQ ID NO:184), miR-379 (SEQ ID NO:186), miR-411 (SEQ ID NO:124), miR-539 (SEQ ID NO:189), miR-543 (SEQ ID NO:133), miR-664 (SEQ ID NO: 152), miR-758 (SEQ ID NO:190), miR-9 (SEQ ID NO:192), miR-98 (SEQ ID NO:193), miR-99b (SEQ ID NO:122); miR-668 (SEQ ID NO:194), miR-122 (SEQ ID NO:22), miR-1227 (SEQ ID NO:157), miR-26a-2# (SEQ ID NO:82), miR-34a (SEQ ID NO:166), miR-551b# (SEQ ID NO:172), miR-1183 (SEQ ID NO:197) and miR-892b (SEQ ID NO:248), and combinations thereof, and the levels relative to a predetermined criterion or range are increased or decreased as disclosed herein.

In any of the embodiments described herein, the one, two, three, four, five, six, seven or more microRNA are selected from the group consisting of let-7b (SEQ ID NO:76), miR-106b# (SEQ ID NO:92), miR-1249 (SEQ ID NO:200), miR-145 (SEQ ID NO:207), miR-151-5P (SEQ ID NO:111), miR-154# (SEQ ID NO:139), miR-15a (SEQ ID NO: 208), miR-181a (SEQ ID NO:66), miR-181c (SEQ ID NO:209), miR-18a# (SEQ ID NO: 211), miR-194 (SEQ ID NO:115), miR-199a-5p (SEQ ID NO:81), miR-199b-5p (SEQ ID NO:213), miR-20a (SEQ ID NO:89), miR-23b (SEQ ID NO:222), miR-30e-3p (SEQ ID NO:224), miR-324-5p (SEQ ID NO:73), miR-411# (SEQ ID NO:147), miR-431 (SEQ ID NO:227), miR-487a (SEQ ID NO:231), miR-493 (SEQ ID NO:232), miR-494 (SEQ ID NO:233), miR-495 (SEQ ID NO:102), miR-505# (SEQ ID NO:235), miR-520a-3p (SEQ ID NO:188), miR-744# (SEQ ID NO:245), miR-769-5p (SEQ ID NO:246), let-7a# (SEQ ID NO:155), miR-10b# (SEQ ID NO:5), miR-1183 (SEQ ID NO:197), miR-1233 (SEQ ID NO:198), miR-1247 (SEQ ID NO:199), miR-1260 (SEQ ID NO:47), miR-1270 (SEQ ID NO:201), miR-1274A (SEQ ID NO:202), miR-1275 (SEQ ID NO:203), miR-128 (SEQ ID NO:158), miR-1290 (SEQ ID NO:34), miR-1298 (SEQ ID NO:204), miR-1303 (SEQ ID NO:3), miR-130a (SEQ ID NO:112), miR-135b (SEQ ID NO:205), miR-138 (SEQ ID NO: 206), miR-140-5p (SEQ ID NO:160), miR-141 (SEQ ID NO:161), miR-142-5p (SEQ ID NO:43), miR-146b-5p (SEQ ID NO:24), miR-148a (SEQ ID NO:91), miR-150 (SEQ ID NO:27), miR-152 (SEQ ID NO:130), miR-15a# (SEQ ID NO:64), miR-185 (SEQ ID NO:163), miR-186 (SEQ ID NO:210), miR-193a-3p (SEQ ID NO:212), miR-193b (SEQ ID NO:12), miR-197 (SEQ ID NO:10), miR-19a (SEQ ID NO:74), miR-200a (SEQ ID NO:214), miR-205 (SEQ ID NO:215), miR-206 (SEQ ID NO:23), miR-20b (SEQ ID NO:216), miR-21 (SEQ ID NO:51), miR-21# (SEQ ID NO:141), miR-214 (SEQ ID NO:217), miR-214# (SEQ ID NO:218), miR-218 (SEQ ID NO:219), miR-220b (SEQ ID NO:220), miR-223 (SEQ ID NO:221), miR-30a-3p (SEQ ID NO:223), miR-30a-5p (SEQ ID NO:164), miR-30d (SEQ ID NO:165), miR-320 (SEQ ID NO:37), miR-324-3p (SEQ ID NO:107), miR-326 (SEQ ID NO:225), miR-335 (SEQ ID NO:119), miR-338-5P (SEQ ID NO:15), miR-346 (SEQ ID NO:226), miR-34a# (SEQ ID NO:13), miR-362-3p (SEQ ID NO:96), miR-375 (SEQ ID NO:8), miR-378 (SEQ ID NO:167), miR-425 (SEQ ID NO: 168), miR-450a (SEQ ID NO:228), miR-450b-5p (SEQ ID NO:229), miR-455-5p (SEQ ID NO:230), miR-501-5p (SEQ ID NO:234) miR-511 (SEQ ID NO:236), miR-518d-3p (SEQ ID NO:237), miR-518e (SEQ ID NO:238), miR-518f (SEQ ID NO:239), miR-548a-3p (SEQ ID NO:14), miR-548c-3p (SEQ ID NO:240), miR-570 (SEQ ID NO:241), miR-571 (SEQ ID NO:242), miR-574-3p (SEQ ID NO:4), miR-577 (SEQ ID NO:243), miR-579 (SEQ ID NO:170), miR-618 (SEQ ID NO:244), miR-885-5p (SEQ ID NO:247), miR-9 (SEQ ID NO:249), miR-95 (SEQ ID NO:250), let-7a (SEQ ID NO:173), let-7d (SEQ ID NO:45), let-7e (SEQ ID NO:93), let-7f (SEQ ID NO:174), let-7g (SEQ ID NO:72); miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-379 (SEQ ID NO:186), miR-411 (SEQ ID NO:124), miR-454 (SEQ ID NO:187), miR-539 (SEQ ID NO:189), miR-543 (SEQ ID NO:133), miR-548J (SEQ ID NO:148), miR-664 (SEQ ID NO:152), miR-758 (SEQ ID NO:190), miR-766 (SEQ ID NO:191), miR-9 (SEQ ID NO:192), miR-98 (SEQ ID NO:193), miR-99b (SEQ ID NO:122); miR-668 (SEQ ID NO:194), miR-106a (SEQ ID NO:156), miR-122 (SEQ ID NO:22), miR-1227 (SEQ ID NO:157), miR-132 (SEQ ID NO:159), miR-146a (SEQ ID NO:21), miR-17 (SEQ ID NO: 162), miR-222 (SEQ ID NO:16), miR-26a-2# (SEQ ID NO:82), miR-345 (SEQ ID NO: 18), miR-34a (SEQ ID NO:166), miR-429 (SEQ ID NO:169), miR-590-5p (SEQ ID NO: 104), miR-598 (SEQ ID NO:110), miR-551b# (SEQ ID NO:172), miR-1183 (SEQ ID NO: 197) and miR-892b, (SEQ ID NO:248) and combinations thereof, and the levels relative to a predetermined criterion or range are increased or decreased as disclosed herein.

In any of the embodiments described herein, the one, two, three, four, five, six, seven or more microRNA are selected from the group consisting of miR-106b# (SEQ ID NO: 92), miR-1249 (SEQ ID NO:200), miR-145 (SEQ ID NO:207), miR-199a-5p (SEQ ID NO: 81), miR-23b (SEQ ID NO:222), miR-29b (SEQ ID NO:125), miR-431 (SEQ ID NO:227), miR-487a (SEQ ID NO:231), miR-493 (SEQ ID NO:232), miR-494 (SEQ ID NO:233), miR-495 (SEQ ID NO:102), miR-744# (SEQ ID NO:245), miR-154# (SEQ ID NO:139), miR-27b# (SEQ ID NO:180), miR-374a (SEQ ID NO:68), miR-411# (SEQ ID NO:147), miR-454 (SEQ ID NO:187), miR-520a-3p (SEQ ID NO:188), miR-548J (SEQ ID NO:148), miR-10b# (SEQ ID NO:5), miR-1183 (SEQ ID NO:197), miR-1233 (SEQ ID NO:198), miR-1247 (SEQ ID NO:199), miR-1260 (SEQ ID NO:47), miR-1270 (SEQ ID NO:201), miR-1274A (SEQ ID NO:202), miR-1275 (SEQ ID NO:203), miR-1290 (SEQ ID NO:34), miR-1298 (SEQ ID NO:204), miR-1303 (SEQ ID NO:3), miR-135b (SEQ ID NO:205), miR-138 (SEQ ID NO:206), miR-193a-3p (SEQ ID NO:212), miR-193b (SEQ ID NO:12), miR-197 (SEQ ID NO:10), miR-205 (SEQ ID NO:215), miR-206 (SEQ ID NO:23), miR-214# (SEQ ID NO:218), miR-214 (SEQ ID NO:217), miR-218 (SEQ ID NO:219), miR-220b (SEQ ID NO:220), miR-223 (SEQ ID NO:221), miR-338-5P (SEQ ID NO:15), miR-346 (SEQ ID NO:226), miR-34a# (SEQ ID NO:13), miR-375 (SEQ ID NO:8), miR-429 (SEQ ID NO:169), miR-450a (SEQ ID NO:228), miR-450b-5p (SEQ ID NO:229), miR-455-5p (SEQ ID NO:230), miR-501-5p (SEQ ID NO:234), miR-511 (SEQ ID NO:236), miR-518d-3p (SEQ ID NO:237), miR-518e (SEQ ID NO:238), miR-518f (SEQ ID NO: 239), miR-548a-3p (SEQ ID NO:14), miR-548c-3p (SEQ ID NO:240), miR-570 (SEQ ID NO:241), miR-571 (SEQ ID NO:242), miR-577 (SEQ ID NO:243), miR-618 (SEQ ID NO: 244), miR-885-5p (SEQ ID NO:247), miR-95 (SEQ ID NO:250), let-7a# (SEQ ID NO:155), miR-130a (SEQ ID NO:112), miR-132 (SEQ ID NO:159), miR-141 (SEQ ID NO:161), miR-148a (SEQ ID NO:91), miR-150 (SEQ ID NO:27), miR-345 (SEQ ID NO:18), miR-362-3p (SEQ ID NO:96), miR-378 (SEQ ID NO:167), miR-579 (SEQ ID NO:170), miR-598 (SEQ ID NO:110), let-7a (SEQ ID NO:173), let-7d (SEQ ID NO:45), let-7e (SEQ ID NO:93), let-7f (SEQ ID NO:174), let-7g (SEQ ID NO:72); miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO: 69), miR-148b# (SEQ ID NO:128), miR-15b (SEQ ID NO:56), miR-181a-2# (SEQ ID NO: 177), miR-190 (SEQ ID NO:63), miR-196b (SEQ ID NO:140), miR-19b-1# (SEQ ID NO: 178), miR-200c (SEQ ID NO:179), miR-20a# (SEQ ID NO:75), miR-24-2# (SEQ ID NO: 120), miR-26a (SEQ ID NO:70), miR-27b# (SEQ ID NO:180), miR-28-5p (SEQ ID NO: 90), miR-29b (SEQ ID NO:125), miR-301a (SEQ ID NO:67), miR-30b (SEQ ID NO:42), miR-30c (SEQ ID NO:52), miR-331-3p (SEQ ID NO:181), miR-339-5p (SEQ ID NO: 182), miR-340# (SEQ ID NO:127), miR-362-5p (SEQ ID NO:183), miR-370 (SEQ ID NO: 184), miR-374a (SEQ ID NO:68), miR-374b (SEQ ID NO:185), miR-379 (SEQ ID NO: 186), miR-411 (SEQ ID NO:124), miR-454 (SEQ ID NO:187), miR-539 (SEQ ID NO:189), miR-543 (SEQ ID NO:133), miR-548J (SEQ ID NO:148), miR-664 (SEQ ID NO:152), miR-758 (SEQ ID NO:190), miR-766 (SEQ ID NO:191), miR-9 (SEQ ID NO:192), miR-98 (SEQ ID NO:193), miR-99b (SEQ ID NO:122); miR-668 (SEQ ID NO:194), miR-106a (SEQ ID NO:156), miR-122 (SEQ ID NO:22), miR-1227 (SEQ ID NO:157), miR-132 (SEQ ID NO:159), miR-146a (SEQ ID NO:21), miR-17 (SEQ ID NO:162), miR-222 (SEQ ID NO:16), miR-26a-2# (SEQ ID NO:82), miR-345 (SEQ ID NO:18), miR-34a (SEQ ID NO:166), miR-429 (SEQ ID NO:169), miR-590-5p (SEQ ID NO:104), miR-598 (SEQ ID NO:110), miR-551b# (SEQ ID NO:172), miR-1183 (SEQ ID NO:197) and miR-892b, (SEQ ID NO:248) and combinations thereof, and the levels relative to a predetermined criterion or range are increased or decreased as disclosed herein.

In any of the embodiments described herein, the levels of 1, 2, 3, 4, 5, 6, 7, 8, 9, 10, 11, 12, 13, 14, 15, 16, 17, 18, 19, 20 21, 22, 23, 24, 25, 26, 27, 28, 29, 30, 35, 40 or more different microRNAs is detected. When levels of multiple different microRNAs are detected, some of which may be increased or decreased relative to a predetermined criterion or range, the various increased or decreased levels form an expression pattern.

In one variation, the expression pattern or increased level or presence of one, two, three, four, five, six, seven or more microRNA is detected, relative to a predetermined criterion, and is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-130a (SEQ ID NO:112), miR-142-5p (SEQ ID NO:43), miR-148a (SEQ ID NO:91), miR-152 (SEQ ID NO:130), miR-15a# (SEQ ID NO:64), miR-19a (SEQ ID NO: 74), miR-21 (SEQ ID NO:51), miR-21# (SEQ ID NO:141), miR-26a-2# (SEQ ID NO:82), miR-324-3p (SEQ ID NO:107), miR-335 (SEQ ID NO:119), miR-362-3p (SEQ ID NO:96, miR-590-5p (SEQ ID NO:104), miR-598 (SEQ ID NO:110), let-7a# (SEQ ID NO:155), miR-106a (SEQ ID NO:156), miR-1227 (SEQ ID NO:157), miR-128 (SEQ ID NO:158), miR-132 (SEQ ID NO:159), miR-140-5p (SEQ ID NO:160), miR-141 (SEQ ID NO:161), miR-17 (SEQ ID NO:162), miR-185 (SEQ ID NO:163), miR-30a-5p (SEQ ID NO:164), miR-30d (SEQ ID NO:165), miR-34a (SEQ ID NO:166), miR-378 (SEQ ID NO:167), miR-425 (SEQ ID NO:168), miR-429 (SEQ ID NO:169), miR-579 (SEQ ID NO:170), miR-523 (SEQ ID NO:171), miR-551b# (SEQ ID NO:172), and combinations thereof.

In one variation, the expression pattern or increased level or presence of one, two, three, four, five, six, seven or more microRNA is detected, relative to a predetermined criterion, and is indicative of a diagnosis of IPF, and the one or more microRNAs is selected from the group consisting of miR-222 (SEQ ID NO:16), miR-345 (SEQ ID NO:18), miR-146a (SEQ ID NO:21), miR-122 (SEQ ID NO:22), miR-146b-5p (SEQ ID NO:24), miR-1300 (SEQ ID NO:25), miR-150 (SEQ ID NO:27), miR-130a (SEQ ID NO:112), miR-142-5p (SEQ ID NO:43), miR-148a (SEQ ID NO:91), miR-152 (SEQ ID NO:130), miR-15a# (SEQ ID NO:64), miR-19a (SEQ ID NO:74), miR-21 (SEQ ID NO:51), miR-21# (SEQ ID NO:141), miR-26a-2# (SEQ ID NO:82), miR-324-3p (SEQ ID NO:107), miR-335 (SEQ ID NO:119), miR-362-3p (SEQ ID NO:96, miR-590-5p (SEQ ID NO:104), miR-598 (SEQ ID NO:110), let-7a# (SEQ ID NO:155), miR-106a (SEQ ID NO:156), miR-1227 (SEQ ID NO: 157), miR-128 (SEQ ID NO:158), miR-132 (SEQ ID NO:159), miR-140-5p (SEQ ID NO: 160), miR-141 (SEQ ID NO:161), miR-17 (SEQ ID NO:162), miR-185 (SEQ ID NO:163), miR-30a-5p (SEQ ID NO:164), miR-30d (SEQ ID NO:165), miR-34a (SEQ ID NO:166), miR-378 (SEQ ID NO:167), miR-425 (SEQ ID NO:168), miR-429 (SEQ ID NO:169), miR-579 (SEQ ID NO:170), miR-523 (SEQ ID NO:171), miR-551b# (SEQ ID NO:172), and combinations thereof.

In another variation, the expression pattern or increased level or presence of one, two, three, four, five, six, seven or more microRNA is detected, relative to a predetermined criterion, and is indicative of a diagnosis of IPF, and the one or more microRNAs is selected from the group consisting of let-7b (SEQ ID NO:76), miR-106a (SEQ ID NO:156), miR-10b# (SEQ ID NO:5), miR-1183 (SEQ ID NO:197), miR-122 (SEQ ID NO:22), miR-1227 (SEQ ID NO:157), miR-1233 (SEQ ID NO:198), miR-1247 (SEQ ID NO:199), miR-1270 (SEQ ID NO:201), miR-1274A (SEQ ID NO:202), miR-1275 (SEQ ID NO:203), miR-1290 (SEQ ID NO:34), miR-1298 (SEQ ID NO:204), miR-1303 (SEQ ID NO:3), miR-132 (SEQ ID NO:159), miR-135b (SEQ ID NO:205), miR-138 (SEQ ID NO:206), miR-146a (SEQ ID NO:21), miR-17 (SEQ ID NO:162), miR-186 (SEQ ID NO:210), miR-193a-3p (SEQ ID NO:212), miR-193b (SEQ ID NO:12), miR-197 (SEQ ID NO:10), miR-200a (SEQ ID NO:214), miR-205 (SEQ ID NO:215), miR-206 (SEQ ID NO:23), miR-20b (SEQ ID NO:216), miR-214 (SEQ ID NO:217), miR-214# (SEQ ID NO:218), miR-218 (SEQ ID NO:219), miR-220b (SEQ ID NO:220), miR-222 (SEQ ID NO:16), miR-223 (SEQ ID NO: 221), miR-26a-2# (SEQ ID NO:82), miR-30a-3p (SEQ ID NO:223), miR-320 (SEQ ID NO: 37), miR-326 (SEQ ID NO:225), miR-338-5P (SEQ ID NO:15), miR-345 (SEQ ID NO: 18), miR-346 (SEQ ID NO:226), miR-34a (SEQ ID NO:166), miR-34a# (SEQ ID NO:13), miR-375 (SEQ ID NO:8), miR-429 (SEQ ID NO:169), miR-450a (SEQ ID NO:228), miR-450b-5p (SEQ ID NO:229), miR-455-5p (SEQ ID NO:230), miR-501-5p (SEQ ID NO: 234), miR-511 (SEQ ID NO:236), miR-518d-3p (SEQ ID NO:237), miR-518e (SEQ ID NO:238), miR-518f (SEQ ID NO:239), miR-548a-3p (SEQ ID NO:14), miR-548c-3p (SEQ ID NO:240), miR-570 (SEQ ID NO:241), miR-571 (SEQ ID NO:242), miR-574-3p (SEQ ID NO:4), miR-577 (SEQ ID NO:243), miR-590-5p (SEQ ID NO:104), miR-598 (SEQ ID NO:110), miR-618 (SEQ ID NO:244), miR-885-5p (SEQ ID NO:247), miR-9# (SEQ ID NO:249), miR-95 (SEQ ID NO:250), miR-26a-2# (SEQ ID NO:82), miR-551b# (SEQ ID NO:172), let-7a# (SEQ ID NO:155), miR-1260 (SEQ ID NO:47), miR-128 (SEQ ID NO: 158), miR-130a (SEQ ID NO:112), miR-140-5p (SEQ ID NO:160), miR-141 (SEQ ID NO: 161), miR-142-5p (SEQ ID NO:43), miR-146b-5p (SEQ ID NO:24), miR-148a (SEQ ID NO:91), miR-150 (SEQ ID NO:27), miR-152 (SEQ ID NO:130), miR-15a# (SEQ ID NO:64), miR-185 (SEQ ID NO:163), miR-19a (SEQ ID NO:74), miR-21 (SEQ ID NO:51), miR-21# (SEQ ID NO:141), miR-30a-5p (SEQ ID NO:164), miR-30d (SEQ ID NO:165), miR-324-3p (SEQ ID NO:107), miR-335 (SEQ ID NO:119), miR-362-3p (SEQ ID NO:96), miR-378 (SEQ ID NO:167), miR-425 (SEQ ID NO:168), miR-579 (SEQ ID NO:170), and combinations thereof.

In some embodiments, the expression pattern or increased level or presence of one, two, three, four, five, six, seven or more microRNA is detected, relative to a predetermined criterion, and is indicative of a diagnosis of IPF, and the one or more microRNAs is selected from the group consisting of miR-122 (SEQ ID NO:22), miR-1227 (SEQ ID NO:157), miR-26a-2# (SEQ ID NO:82), miR-34a (SEQ ID NO:166), miR-551b# (SEQ ID NO:172), and combinations thereof.

In some embodiments, the expression pattern or increased level or presence of one, two, three, four, five, six, seven or more microRNA is detected, relative to a predetermined criterion, and is indicative of a diagnosis of IPF, and the one or more microRNAs is selected from the group consisting of miR-106a (SEQ ID NO:156), miR-122 (SEQ ID NO:22), miR-1227 (SEQ ID NO:157), miR-132 (SEQ ID NO:159), miR-146a (SEQ ID NO:21), miR-17 (SEQ ID NO:162), miR-222 (SEQ ID NO:16), miR-26a-2# (SEQ ID NO:82), miR-345 (SEQ ID NO:18), miR-34a (SEQ ID NO:166), miR-429 (SEQ ID NO:169), miR-590-5p (SEQ ID NO:104), miR-598 (SEQ ID NO:110), miR-551b# (SEQ ID NO:172), and combinations thereof.

In some embodiments, the expression pattern or increased level or presence of one, two, three, four, five, six, seven or more microRNA is detected, relative to a predetermined criterion, and is indicative of a diagnosis of IPF, and the one or more microRNAs is selected from the group consisting of miR-10b# (SEQ ID NO:5), miR-1183 (SEQ ID NO:197), miR-1233 (SEQ ID NO:198), miR-1247 (SEQ ID NO:199), miR-1260 (SEQ ID NO:47), miR-1270 (SEQ ID NO:201), miR-1274A (SEQ ID NO:202), miR-1275 (SEQ ID NO:203), miR-1290 (SEQ ID NO:34), miR-1298 (SEQ ID NO:204), miR-1303 (SEQ ID NO:3), miR-135b (SEQ ID NO:205), miR-138 (SEQ ID NO:206), miR-193a-3p (SEQ ID NO: 212), miR-193b (SEQ ID NO:12), miR-197 (SEQ ID NO:10), miR-205 (SEQ ID NO:215), miR-206 (SEQ ID NO:23), miR-214# (SEQ ID NO:218), miR-214 (SEQ ID NO:217), miR-218 (SEQ ID NO:219), miR-220b (SEQ ID NO:220), miR-223 (SEQ ID NO:221), miR-338-5P (SEQ ID NO:15), miR-346 (SEQ ID NO:226), miR-34a# (SEQ ID NO:13), miR-375 (SEQ ID NO:8), miR-429 (SEQ ID NO:169), miR-450a (SEQ ID NO:228), miR-450b-5p (SEQ ID NO:229), miR-455-5p (SEQ ID NO:230), miR-501-5p (SEQ ID NO:234), miR-511 (SEQ ID NO:236), miR-518d-3p (SEQ ID NO:237), miR-518e (SEQ ID NO:238), miR-518f (SEQ ID NO:239), miR-548a-3p (SEQ ID NO:14), miR-548c-3p (SEQ ID NO: 240), miR-570 (SEQ ID NO:241), miR-571 (SEQ ID NO:242), miR-577 (SEQ ID NO:243), miR-618 (SEQ ID NO:244), miR-885-5p (SEQ ID NO:247), miR-95 (SEQ ID NO:250), let-7a# (SEQ ID NO:155), miR-130a (SEQ ID NO:112), miR-132 (SEQ ID NO:159), miR-141 (SEQ ID NO:161), miR-148a (SEQ ID NO:91), miR-150 (SEQ ID NO:27), miR-345 (SEQ ID NO:18), miR-362-3p (SEQ ID NO:96), miR-378 (SEQ ID NO:167), miR-579 (SEQ ID NO:170), miR-598 (SEQ ID NO:110), and combinations thereof.

In some embodiments, the expression pattern or increased level or presence of one, two, three, four, five, six, seven or more microRNA is detected, relative to a predetermined criterion, and is indicative of a diagnosis of IPF, and the one or more microRNAs is selected from the group consisting of let-7a# (SEQ ID NO:155), let-7b (SEQ ID NO:76), miR-10b# (SEQ ID NO:5), miR-1183 (SEQ ID NO:197), miR-1233 (SEQ ID NO:198), miR-1247 (SEQ ID NO:199), miR-1260 (SEQ ID NO:47), miR-1270 (SEQ ID NO:201), miR-1274A (SEQ ID NO:202), miR-1275 (SEQ ID NO:203), miR-128 (SEQ ID NO:158), miR-1290 (SEQ ID NO:34), miR-1298 (SEQ ID NO:204), miR-1303 (SEQ ID NO:3), miR-130a (SEQ ID NO:112), miR-135b (SEQ ID NO:205), miR-138 (SEQ ID NO:206), miR-140-5p (SEQ ID NO:160), miR-141 (SEQ ID NO:161), miR-142-5p (SEQ ID NO:43), miR-146b-5p (SEQ ID NO:24), miR-148a (SEQ ID NO:91), miR-150 (SEQ ID NO:27), miR-152 (SEQ ID NO:130), miR-15a# (SEQ ID NO:64), miR-185 (SEQ ID NO:163), miR-186 (SEQ ID NO:210), miR-193a-3p (SEQ ID NO:212), miR-193b (SEQ ID NO:12), miR-197 (SEQ ID NO:10), miR-19a (SEQ ID NO:74), miR-200a (SEQ ID NO:214), miR-205 (SEQ ID NO:215), miR-206 (SEQ ID NO:23), miR-20b (SEQ ID NO:216), miR-21 (SEQ ID NO: 51), miR-21# (SEQ ID NO:141), miR-214 (SEQ ID NO:217), miR-214# (SEQ ID NO:218), miR-218 (SEQ ID NO:219), miR-220b (SEQ ID NO:220), miR-223 (SEQ ID NO:221), miR-30a-3p (SEQ ID NO:223), miR-30a-5p (SEQ ID NO:164), miR-30d (SEQ ID NO: 165), miR-320 (SEQ ID NO:37), miR-324-3p (SEQ ID NO:107), miR-326 (SEQ ID NO:225), miR-335 (SEQ ID NO:119), miR-338-5P (SEQ ID NO:15), miR-346 (SEQ ID NO:226), miR-34a# (SEQ ID NO:13), miR-362-3p (SEQ ID NO:96), miR-375 (SEQ ID NO:8), miR-378 (SEQ ID NO:167), miR-425 (SEQ ID NO:168), miR-450a (SEQ ID NO:228), miR-450b-5p (SEQ ID NO:229), miR-455-5p (SEQ ID NO:230), miR-501-5p (SEQ ID NO:234) miR-511 (SEQ ID NO:236), miR-518d-3p (SEQ ID NO:237), miR-518e (SEQ ID NO:238), miR-518f (SEQ ID NO:239), miR-548a-3p (SEQ ID NO:14), miR-548c-3p (SEQ ID NO:240), miR-570 (SEQ ID NO:241), miR-571 (SEQ ID NO:242), miR-574-3p (SEQ ID NO:4), miR-577 (SEQ ID NO:243), miR-579 (SEQ ID NO:170), miR-618 (SEQ ID NO:244), miR-885-5p (SEQ ID NO:247), miR-9# (SEQ ID NO:249), miR-95 (SEQ ID NO:250) and combinations thereof.

In some embodiments, the one, two, three, four, five, six, seven or more microRNAs is selected from the group miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), and combinations thereof.

In some embodiments, the one, two, three, four, five, six, seven or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13) and miR-548a-3p (SEQ ID NO:14) and combinations thereof.

In some embodiments, the one, two, three, four, five, six, seven or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), and combinations thereof.

In another variation, the expression pattern, or decreased level or absence of one or more microRNA is detected, relative to a predetermined criterion, and is indicative of a diagnosis of IPF, and the one, two, three, four, five, six, seven or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2 (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNAs is selected from the group consisting of miR-1244 (SEQ ID NO:20), miR-142-3p (SEQ ID NO:38), miR-30b (SEQ ID NO:42), let-7d (SEQ ID NO:45), miR-30c (SEQ ID NO:52), miR-15b (SEQ ID NO:56), miR-190 (SEQ ID NO:63), miR-301a (SEQ ID NO:67), miR-374a (SEQ ID NO:68), miR-144# (SEQ ID NO:69), miR-26a (SEQ ID NO:70), let-7g (SEQ ID NO:72), miR-20a# (SEQ ID NO:75), miR-28-5p (SEQ ID NO:90), let-7e (SEQ ID NO:93), miR-127-3p (SEQ ID NO:101), miR-103 (SEQ ID NO:105), miR-24-2# (SEQ ID NO:120), miR-99b (SEQ ID NO:122), miR-411 (SEQ ID NO:124), miR-29b (SEQ ID NO:125), miR-340# (SEQ ID NO:127), miR-148b# (SEQ ID NO:128), miR-543 (SEQ ID NO:133), miR-154# (SEQ ID NO:139), miR-196b (SEQ ID NO:140), miR-411# (SEQ ID NO:147), miR-548J (SEQ ID NO:148), miR-664 (SEQ ID NO:152), let-7a (SEQ ID NO: 173), let-7f (SEQ ID NO:174), miR-107 (SEQ ID NO:175), miR-125a-5p (SEQ ID NO: 176), miR-181a-2# (SEQ ID NO:177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO: 194), miR-1256 (SEQ ID NO:195), miR-299-5p (SEQ ID NO:196), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNA are selected from the group consisting of let-7a (SEQ ID NO:173), let-7e (SEQ ID NO:93), let-7f (SEQ ID NO:174), let-7g (SEQ ID NO:72), miR-107 (SEQ ID NO:175), miR-1244 (SEQ ID NO:20), miR-1256 (SEQ ID NO:195), miR-127-3p (SEQ ID NO:101), miR-144 (SEQ ID NO:69), miR-148b# (SEQ ID NO:128), miR-154# (SEQ ID NO:139), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-27b# (SEQ ID NO:180), miR-28-5p (SEQ ID NO:90), miR-299-5p (SEQ ID NO:196), miR-301a (SEQ ID NO:67), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-411 (SEQ ID NO:124), miR-411# (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO:190), miR-766 (SEQ ID NO:191), miR-9 (SEQ ID NO:192), miR-98 (SEQ ID NO:193), miR-99b (SEQ ID NO:122), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNA are selected from the group consisting of let-7d (SEQ ID NO:45), miR-103 (SEQ ID NO:105), miR-125a-5p (SEQ ID NO:176), miR-142-3p (SEQ ID NO:38), miR-26a (SEQ ID NO:70), miR-29b (SEQ ID NO:125), miR-30b (SEQ ID NO:42), miR-30c (SEQ ID NO:52), miR-379 (SEQ ID NO:186), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNA are selected from the group consisting of miR-18a (SEQ ID NO:39), miR-26b (SEQ ID NO:40), miR-106b (SEQ ID NO:41), miR-29c (SEQ ID NO:44), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-148b (SEQ ID NO:53), miR-27b (SEQ ID NO:54), miR-15b# (SEQ ID NO:55), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-27a (SEQ ID NO:65), miR-181a (SEQ ID NO:66), miR-320B (SEQ ID NO: 71), miR-324-5p (SEQ ID NO:73), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-106b# (SEQ ID NO:92), miR-25 (SEQ ID NO:94), miR-656 (SEQ ID NO:95), miR-340 (SEQ ID NO:97), miR-451 (SEQ ID NO:98), miR-423-5p (SEQ ID NO:99), miR-652 (SEQ ID NO:100), miR-495 (SEQ ID NO:102), miR-328 (SEQ ID NO:103), miR-19b (SEQ ID NO:106), miR-145 (SEQ ID NO:108), miR-199a-3p (SEQ ID NO:109), miR-151-5P (SEQ ID NO:111), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-130b (SEQ ID NO:121), miR-195 (SEQ ID NO:123), miR-576-3p (SEQ ID NO:126), miR-212 (SEQ ID NO:129), miR-143 (SEQ ID NO:131), dme-miR-7 (SEQ ID NO:132), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-551b (SEQ ID NO:149), miR-616 (SEQ ID NO:150), miR-638 (SEQ ID NO:151), miR-889 (SEQ ID NO:153), rno-miR-29c# (SEQ ID NO:154), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNA are selected from the group consisting of let-7a (SEQ ID NO:173), let-7d (SEQ ID NO:45), let-7e (SEQ ID NO:93), let-7f (SEQ ID NO:174), miR-103 (SEQ ID NO:105), miR-142-3p (SEQ ID NO:38), miR-144# (SEQ ID NO:69), miR-148b# (SEQ ID NO:128), miR-154 (SEQ ID NO:139), miR-15b (SEQ ID NO:56), miR-181a-2# (SEQ ID NO:177), miR-190 (SEQ ID NO:63), miR-20a# (SEQ ID NO:75), miR-24-2# (SEQ ID NO:120), miR-26a (SEQ ID NO:70), miR-28-5p (SEQ ID NO:90), miR-30b (SEQ ID NO:42), miR-30c (SEQ ID NO:52), miR-331-3p (SEQ ID NO:181), miR-340# (SEQ ID NO:127), miR-362-5p (SEQ ID NO:183), miR-374a (SEQ ID NO:68), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-758 (SEQ ID NO:190), miR-1256 (SEQ ID NO:195), miR-299-5p (SEQ ID NO:196), miR-668 (SEQ ID NO:194), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNA are selected from the group consisting of let-7g (SEQ ID NO:72), miR-107 (SEQ ID NO:175), miR-1244 (SEQ ID NO:20), miR-125a-5p (SEQ ID NO:176), miR-127-3p (SEQ ID NO:101), miR-196b (SEQ ID NO:140), miR-19b-1# (SEQ ID NO:178), miR-200c (SEQ ID NO:179), miR-27b# (SEQ ID NO:180), miR-29b (SEQ ID NO:125), miR-301a (SEQ ID NO:67), miR-339-5p (SEQ ID NO:182), miR-370 (SEQ ID NO:184), miR-374b (SEQ ID NO:185), miR-539 (SEQ ID NO:189), miR-543 (SEQ ID NO:133), miR-548J (SEQ ID NO: 148), miR-664 (SEQ ID NO:152), miR-766 (SEQ ID NO:191), miR-9 (SEQ ID NO:192), miR-98 (SEQ ID NO:193), miR-99b (SEQ ID NO:122), and combinations thereof.

In some embodiments, the expression pattern, or decreased level or absence of one or more microRNA is detected, relative to a predetermined criterion, is indicative of a diagnosis of progressive IPF, and the one or more microRNAs is selected from the group consisting of miR-1183 (SEQ ID NO:197) and miR-892b (SEQ ID NO:248). In some embodiments, subjects identified as having progressive IPF are administered an anti-fibrotic agent described herein.

In some embodiments, the expression pattern, or decreased level or absence of one or more microRNA is detected, relative to a predetermined criterion, and is indicative of a diagnosis of IPF, and the one, two, three, four, five, six, seven or more microRNAs is selected from the group consisting of let-7a (SEQ ID NO:173), let-7d (SEQ ID NO:45), let-7e (SEQ ID NO:93), let-7f (SEQ ID NO:174), let-7g (SEQ ID NO:72), miR-103 (SEQ ID NO:105), miR-106b# (SEQ ID NO:92), miR-107 (SEQ ID NO:175), miR-1244 (SEQ ID NO:20), miR-1249 (SEQ ID NO:200), miR-125a-5p (SEQ ID NO:176), miR-1260 (SEQ ID NO:47), miR-127-3p (SEQ ID NO:101), miR-142-3p (SEQ ID NO:38), miR-144# (SEQ ID NO:69), miR-145 (SEQ ID NO:207), miR-148b# (SEQ ID NO:128), miR-151-5P (SEQ ID NO:111), miR-15a (SEQ ID NO:208), miR-15b (SEQ ID NO:56), miR-181a (SEQ ID NO:66), miR-181a-2# (SEQ ID NO:177), miR-181c (SEQ ID NO:209), miR-18a# (SEQ ID NO:211), miR-190 (SEQ ID NO:63), miR-194 (SEQ ID NO:115), miR-196b (SEQ ID NO: 140), miR-199a-5p (SEQ ID NO:81), miR-199b-5p (SEQ ID NO:213), miR-19b-1# (SEQ ID NO:178), miR-200c (SEQ ID NO:179), miR-20a (SEQ ID NO:89), miR-20a# (SEQ ID NO:75), miR-23b (SEQ ID NO:222), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO:42), miR-30c (SEQ ID NO: 52), miR-30e-3p (SEQ ID NO:224), miR-324-5p (SEQ ID NO:73), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-379 (SEQ ID NO:186), miR-411 (SEQ ID NO:124), miR-431 (SEQ ID NO: 227), miR-454 (SEQ ID NO:187), miR-487a (SEQ ID NO:231), miR-493 (SEQ ID NO: 232), miR-494 (SEQ ID NO:233), miR-495 (SEQ ID NO:102), miR-505# (SEQ ID NO: 235), miR-539 (SEQ ID NO:189), miR-543 (SEQ ID NO:133), miR-548J (SEQ ID NO: 148), miR-664 (SEQ ID NO:152), miR-744# (SEQ ID NO:245), miR-758 (SEQ ID NO: 190), miR-766 (SEQ ID NO:191), miR-769-5p (SEQ ID NO:246), miR-9 (SEQ ID NO: 192), miR-98 (SEQ ID NO:193), miR-99b (SEQ ID NO:122), miR-148b# (SEQ ID NO: 128), miR-668 (SEQ ID NO:194), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNAs is selected from the group consisting of let-7a (SEQ ID NO:173), let-7d (SEQ ID NO:45), let-7e (SEQ ID NO:93), let-7f (SEQ ID NO:174), miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-20a# (SEQ ID NO:75), miR-24-2 (SEQ ID NO:120), miR-26a (SEQ ID NO:70), miR-28-5p (SEQ ID NO:90), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-379 (SEQ ID NO:186), miR-411 (SEQ ID NO:124), miR-539 (SEQ ID NO:189), miR-543 (SEQ ID NO:133), miR-664 (SEQ ID NO:152), miR-758 (SEQ ID NO:190), miR-9 (SEQ ID NO:192), miR-98 (SEQ ID NO:193), miR-99b (SEQ ID NO:122); miR-668 (SEQ ID NO:194), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNAs is selected from the group consisting of let-7a (SEQ ID NO:173), let-7d (SEQ ID NO:45), let-7e (SEQ ID NO:93), let-7f (SEQ ID NO:174), let-7g (SEQ ID NO:72); miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO: 42), miR-30c (SEQ ID NO:52), miR-331-3p (SEQ ID NO:181), miR-339-5p (SEQ ID NO: 182), miR-340# (SEQ ID NO:127), miR-362-5p (SEQ ID NO:183), miR-370 (SEQ ID NO: 184), miR-374a (SEQ ID NO:68), miR-374b (SEQ ID NO:185), miR-379 (SEQ ID NO: 186), miR-411 (SEQ ID NO:124), miR-454 (SEQ ID NO:187), miR-539 (SEQ ID NO:189), miR-543 (SEQ ID NO:133), miR-548J (SEQ ID NO:148), miR-664 (SEQ ID NO:152), miR-758 (SEQ ID NO:190), miR-766 (SEQ ID NO:191), miR-9 (SEQ ID NO:192), miR-98 (SEQ ID NO:193), miR-99b (SEQ ID NO:122); miR-668 (SEQ ID NO:194), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNAs is selected from the group consisting of miR-106b# (SEQ ID NO:92), miR-1249 (SEQ ID NO: 200), miR-145 (SEQ ID NO:207), miR-199a-5p (SEQ ID NO:81), miR-23b (SEQ ID NO: 222), miR-29b (SEQ ID NO:125), miR-431 (SEQ ID NO:227), miR-487a (SEQ ID NO:231), miR-493 (SEQ ID NO:232), miR-494 (SEQ ID NO:233), miR-495 (SEQ ID NO: 102), miR-744# (SEQ ID NO:245), miR-154# (SEQ ID NO:139), miR-27b# (SEQ ID NO: 180), miR-374a (SEQ ID NO:68), miR-411# (SEQ ID NO:147), miR-454 (SEQ ID NO: 187), miR-520a-3p (SEQ ID NO:188), miR-548J (SEQ ID NO:148), and combinations thereof.

In some embodiments, the expression pattern, or decreased level or absence of one or more microRNA is detected, relative to a predetermined criterion, and is indicative of a diagnosis of IPF, and the one, two, three, four, five, six, seven or more microRNAs is selected from the group consisting of miR-106b# (SEQ ID NO:92), miR-1249 (SEQ ID NO: 200), miR-145 (SEQ ID NO:207), miR-151-5P (SEQ ID NO:111), miR-154# (SEQ ID NO: 139), miR-15a (SEQ ID NO:208), miR-181a (SEQ ID NO:66), miR-181c (SEQ ID NO: 209), miR-18a# (SEQ ID NO:211), miR-194 (SEQ ID NO:115), miR-199a-5p (SEQ ID NO:81), miR-199b-5p (SEQ ID NO:213), miR-20a (SEQ ID NO:89), miR-23b (SEQ ID NO:222), miR-30e-3p (SEQ ID NO:224), miR-324-5p (SEQ ID NO:73), miR-411# (SEQ ID NO:147), miR-431 (SEQ ID NO:227), miR-487a (SEQ ID NO:231), miR-493 (SEQ ID NO:232), miR-494 (SEQ ID NO:233), miR-495 (SEQ ID NO:102), miR-505# (SEQ ID NO:235), miR-520a-3p (SEQ ID NO:188), miR-744# (SEQ ID NO:245), miR-769-5p (SEQ ID NO:246), and combinations thereof.

In some embodiments, the one, two, three, four, five, six, seven or more microRNA are selected from the group consisting of miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-197 (SEQ ID NO:10), miR-663B (SEQ ID NO:11), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), let-7d (SEQ ID NO:45), miR-361-5p (SEQ ID NO:48), miR-520e (SEQ ID NO:49), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-17# (SEQ ID NO:58), miR-32 (SEQ ID NO:60), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-20a (SEQ ID NO:89), miR-28-5p (SEQ ID NO:90), miR-148a (SEQ ID NO:91), let-7e (SEQ ID NO:93), miR-656 (SEQ ID NO:95), miR-362-3p (SEQ ID NO:96), miR-423-5p (SEQ ID NO:99), miR-652 (SEQ ID NO:100), miR-127-3p (SEQ ID NO:101), miR-495 (SEQ ID NO:102), miR-590-5p (SEQ ID NO:104), miR-103 (SEQ ID NO:105), miR-324-3p (SEQ ID NO:107), miR-598 (SEQ ID NO:110), miR-130a (SEQ ID NO:112), miR-502-3p (SEQ ID NO:113), miR-194 (SEQ ID NO:115), miR-221 (SEQ ID NO:116), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-411 (SEQ ID NO:124), miR-29b (SEQ ID NO:125), miR-340# (SEQ ID NO:127), miR-148b# (SEQ ID NO:128), miR-212 (SEQ ID NO:129), miR-152 (SEQ ID NO:130), miR-7 (SEQ ID NO:132), miR-543 (SEQ ID NO:133), miR-30d (SEQ ID NO:134), miR-213 (SEQ ID NO:135), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), let-7a# (SEQ ID NO:155), miR-106a (SEQ ID NO: 156), miR-1227 (SEQ ID NO:157), miR-128 (SEQ ID NO:158), miR-132 (SEQ ID NO: 159), miR-140-5p (SEQ ID NO:160), miR-141 (SEQ ID NO:161), miR-17 (SEQ ID NO: 162), miR-185 (SEQ ID NO:163), miR-30a-5p (SEQ ID NO:164), miR-30d (SEQ ID NO: 165), miR-34a (SEQ ID NO:166), miR-378 (SEQ ID NO:167), miR-425 (SEQ ID NO:

168), miR-429 (SEQ ID NO:169), miR-579 (SEQ ID NO:170), miR-523 (SEQ ID NO: 171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO: 177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof.

In some embodiments, the one, two, three, four, five, six, seven or more microRNA are selected from the group consisting of miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-197 (SEQ ID NO:10), miR-663B (SEQ ID NO:11), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-572 (SEQ ID NO:35), miR-886-3p (SEQ ID NO:36), miR-26b (SEQ ID NO:40), miR-142-5p (SEQ ID NO:43), let-7d (SEQ ID NO:45), miR-361-5p (SEQ ID NO:48), miR-520e (SEQ ID NO:49), miR-148b (SEQ ID NO:53), miR-15b# (SEQ ID NO:55), miR-15b (SEQ ID NO:56), miR-17# (SEQ ID NO:58), miR-190 (SEQ ID NO:63), miR-15a# (SEQ ID NO:64), miR-181a (SEQ ID NO:66), miR-301a (SEQ ID NO:67), miR-374a (SEQ ID NO:68), miR-144# (SEQ ID NO:69), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-28-5p (SEQ ID NO:90), miR-148a (SEQ ID NO:91), let-7e (SEQ ID NO:93), miR-656 (SEQ ID NO:95), miR-362-3p (SEQ ID NO:96), miR-652 (SEQ ID NO:100), miR-127-3p (SEQ ID NO:101), miR-495 (SEQ ID NO:102), miR-590-5p (SEQ ID NO:104), miR-598 (SEQ ID NO:110), miR-130a (SEQ ID NO:112), miR-502-3p (SEQ ID NO:113), miR-194 (SEQ ID NO:115), miR-221 (SEQ ID NO:116), miR-335 (SEQ ID NO:119), miR-24-2# (SEQ ID NO:120), miR-130b (SEQ ID NO:121), miR-99b (SEQ ID NO:122), miR-411 (SEQ ID NO:124), miR-340# (SEQ ID NO:127), miR-148b# (SEQ ID NO:128), miR-212 (SEQ ID NO:129), miR-152 (SEQ ID NO:130), miR-7 (SEQ ID NO:132), miR-543 (SEQ ID NO:133), miR-30d (SEQ ID NO:134), miR-213 (SEQ ID NO:135), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), let-7a# (SEQ ID NO: 155), miR-106a (SEQ ID NO:156), miR-1227 (SEQ ID NO:157), miR-140-5p (SEQ ID NO:160), miR-141 (SEQ ID NO:161), miR-17 (SEQ ID NO:162), miR-185 (SEQ ID NO: 163), miR-30a-5p (SEQ ID NO:164), miR-30d (SEQ ID NO:165), miR-34a (SEQ ID NO: 166), miR-378 (SEQ ID NO:167), miR-425 (SEQ ID NO:168), miR-429 (SEQ ID NO: 169), miR-579 (SEQ ID NO:170), miR-523 (SEQ ID NO:171), miR-551b# (SEQ ID NO: 172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO: 178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO: 193), miR-668 (SEQ ID NO:194), miR-1256 (SEQ ID NO:195), miR-299-5p (SEQ ID NO:196), and combinations thereof.

In some embodiments, the one, two, three, four, five, six, seven or more microRNA are selected from the group consisting of let-7a (SEQ ID NO:173), let-7a# (SEQ ID NO: 155), let-7d (SEQ ID NO:45), miR-106a (SEQ ID NO:156), let-7e (SEQ ID NO:93), miR-122 (SEQ ID NO:22), let-7f (SEQ ID NO:174), miR-1227 (SEQ ID NO:157), let-7g (SEQ ID NO:72), miR-128 (SEQ ID NO:158), miR-103(SEQ ID NO:105), miR-130a (SEQ ID NO:112), miR-107 (SEQ ID NO:175), miR-132 (SEQ ID NO:159), miR-1244(SEQ ID NO:20), miR-140-5p (SEQ ID NO:160), miR-1256 (SEQ ID NO:195), miR-141 (SEQ ID NO:161), miR-125a-5p (SEQ ID NO:176), miR-142-5p (SEQ ID NO:43), miR-127-3p (SEQ ID NO:101), miR-146a (SEQ ID NO:21), miR-142-3p (SEQ ID NO:38), miR-146b-5p (SEQ ID NO:24), miR-144# (SEQ ID NO:69), miR-148a (SEQ ID NO:91), miR-148b# (SEQ ID NO:128), miR-150 (SEQ ID NO:27), miR-154# (SEQ ID NO:139), miR-152 (SEQ ID NO:130), miR-15b (SEQ ID NO:56), miR-15a# (SEQ ID NO:64), miR-181a-2 (SEQ ID NO:177), miR-17 (SEQ ID NO:162), miR-190 (SEQ ID NO:63), miR-185 (SEQ ID NO:163), miR-196b (SEQ ID NO:140), miR-19a (SEQ ID NO:74), miR-19b-1# (SEQ ID NO:178), miR-21 (SEQ ID NO:51), miR-200c (SEQ ID NO:179), miR-21# (SEQ ID NO:141), miR-20a# (SEQ ID NO:75), miR-222 (SEQ ID NO:16), miR-24-2# (SEQ ID NO:120), miR-26a-2# (SEQ ID NO:82), miR-26a (SEQ ID NO:70), miR-30a-5p (SEQ ID NO:164), miR-27b# (SEQ ID NO:180), miR-30d (SEQ ID NO:165), miR-28-5p (SEQ ID NO:90), miR-324-3p (SEQ ID NO:107), miR-299-5p (SEQ ID NO:196), miR-335 (SEQ ID NO:119), miR-29b (SEQ ID NO:125), miR-345 (SEQ ID NO:18), miR-301a (SEQ ID NO:67), miR-34a (SEQ ID NO:166), miR-30b (SEQ ID NO:42), miR-362-3p (SEQ ID NO:96), miR-30c (SEQ ID NO:52), miR-378 (SEQ ID NO:167), miR-331-3p (SEQ ID NO:181), miR-425 (SEQ ID NO:168), miR-339-5p (SEQ ID NO:182), miR-429 (SEQ ID NO:169), miR-340# (SEQ ID NO:127), miR-523 (SEQ ID NO:171), miR-362-5p (SEQ ID NO:183), miR-551b# (SEQ ID NO:172), miR-370 (SEQ ID NO:184), miR-579 (SEQ ID NO:170), miR-374a (SEQ ID NO:68), miR-590-5p (SEQ ID NO:104), miR-374b (SEQ ID NO:185), miR-598 (SEQ ID NO:110), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO: 190), miR-766 (SEQ ID NO:191), miR-9 (SEQ ID NO:192), miR-98 (SEQ ID NO:193), miR-99b (SEQ ID NO:122), and combinations thereof.

In some embodiments, the one, two, three, four, five, six, seven or more microRNA are selected from the group consisting of let-7a (SEQ ID NO:173), let-7a# (SEQ ID NO: 155), miR-106a (SEQ ID NO:156), let-7e (SEQ ID NO:93), miR-122 (SEQ ID NO:22), let-7f (SEQ ID NO:174), miR-1227 (SEQ ID NO:157), let-7g (SEQ ID NO:72), miR-130a (SEQ ID NO:112), miR-107 (SEQ ID NO:175), miR-1244(SEQ ID NO:20), miR-140-5p (SEQ ID NO:160), miR-1256 (SEQ ID NO:195), miR-141 (SEQ ID NO:161), miR-125a-5p (SEQ ID NO:176), miR-142-5p (SEQ ID NO:43), miR-127-3p (SEQ ID NO:101), miR-146a (SEQ ID NO:21), miR-146b-5p (SEQ ID NO:24), miR-144# (SEQ ID NO:69), miR-148a (SEQ ID NO:91), miR-148b# (SEQ ID NO:128), miR-154# (SEQ ID NO:139), miR-152 (SEQ ID NO:130), miR-15b (SEQ ID NO:56), miR-15a# (SEQ ID NO:64), miR-181a-2# (SEQ ID NO:177), miR-17 (SEQ ID NO:162), miR-190 (SEQ ID NO:63), miR-185 (SEQ ID NO:163), miR-196b (SEQ ID NO:140), miR-19a (SEQ ID NO:74), miR-19b-1# (SEQ ID NO:178), miR-200c (SEQ ID NO:179), miR-21# (SEQ ID NO:141), miR-20a# (SEQ ID NO:75), miR-222 (SEQ ID NO:16), miR-24-2# (SEQ ID NO:120), miR-26a-2 (SEQ ID NO:82), miR-30a-5p (SEQ ID NO:164), miR-27b# (SEQ ID NO:180), miR-30d (SEQ ID NO:165), miR-28-5p (SEQ ID NO:90), miR-299-5p (SEQ ID NO:196), miR-335 (SEQ ID NO:119), miR-345 (SEQ ID NO:18), miR-301a (SEQ ID NO:67), miR-34a (SEQ ID NO:166), miR-362-3p (SEQ ID NO:96), miR-378 (SEQ ID NO:167), miR-331-3p (SEQ ID NO:181), miR-425 (SEQ ID NO:168), miR-339-5p (SEQ ID NO:182), miR-429 (SEQ ID NO:169), miR-340# (SEQ ID NO:127), miR-523 (SEQ ID NO:171), miR-362-5p (SEQ ID NO:183), miR-551b# (SEQ ID NO:172), miR-370 (SEQ ID NO:184), miR-579 (SEQ ID NO:170), miR-374a (SEQ ID NO:68), miR-590-5p (SEQ ID NO:104), miR-374b (SEQ ID NO:185), miR-598 (SEQ ID NO:110), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO: 190), miR-766 (SEQ ID NO:191), miR-9 (SEQ ID NO:192), miR-98 (SEQ ID NO:193), miR-99b (SEQ ID NO:122), and combinations thereof.

In some embodiments, the one, two, three, four, five, six, seven or more microRNA are selected from the group consisting of let-7a (SEQ ID NO:173), miR-132 (SEQ ID NO: 159), let-7d (SEQ ID NO:45), miR-148a (SEQ ID NO:91), let-7e (SEQ ID NO:93), miR-152 (SEQ ID NO:130), let-7f (SEQ ID NO:174), miR-17 (SEQ ID NO:162), miR-103 (SEQ ID NO:105), miR-185 (SEQ ID NO:163), miR-1256 (SEQ ID NO:195), miR-21 (SEQ ID NO:51), miR-142-3p (SEQ ID NO:38), miR-222 (SEQ ID NO:16), miR-144 (SEQ ID NO:69), miR-345 (SEQ ID NO:18), miR-148b# (SEQ ID NO:128), miR-34a (SEQ ID NO:166), miR-154# (SEQ ID NO:139), miR-523 (SEQ ID NO:171), miR-15b (SEQ ID NO:56), miR-551b# (SEQ ID NO:172), miR-181a-2# (SEQ ID NO:177), miR-590-5p (SEQ ID NO:104), miR-190 (SEQ ID NO:63), miR-20a# (SEQ ID NO:75), miR-24-2# (SEQ ID NO:120), miR-26a (SEQ ID NO:70), miR-28-5p (SEQ ID NO:90), miR-299-5p (SEQ ID NO:196), miR-30b (SEQ ID NO:42), miR-30c (SEQ ID NO:52), miR-331-3p (SEQ ID NO:181), miR-340# (SEQ ID NO:127), miR-362-5p (SEQ ID NO:183), miR-374a (SEQ ID NO:68), miR-379 (SEQ ID NO:186), miR-411 (SEQ ID NO:124), miR-411# (SEQ ID NO:147), miR-454 (SEQ ID NO:187), miR-520a-3p (SEQ ID NO: 188), miR-668 (SEQ ID NO:194), miR-758 (SEQ ID NO:190), and combinations thereof.

In some embodiments, the one, two, three, four, five, six, seven or more microRNA are selected from the group consisting of let-7a (SEQ ID NO:173), miR-148a (SEQ ID NO: 91), let-7e (SEQ ID NO:93), miR-152 (SEQ ID NO:130), let-7f (SEQ ID NO:174), miR-17 (SEQ ID NO:162), miR-185 (SEQ ID NO:163), miR-1256 (SEQ ID NO:195), miR-222 (SEQ ID NO:16), miR-144# (SEQ ID NO:69), miR-345 (SEQ ID NO:18), miR-148b# (SEQ ID NO:128), miR-34a (SEQ ID NO:166), miR-154# (SEQ ID NO:139), miR-523 (SEQ ID NO:171), miR-15b (SEQ ID NO:56), miR-551b# (SEQ ID NO:172), miR-181a-2# (SEQ ID NO:177), miR-590-5p (SEQ ID NO:104), miR-190 (SEQ ID NO:63), miR-20a# (SEQ ID NO:75), miR-24-2# (SEQ ID NO:120), miR-28-5p (SEQ ID NO:90), miR-299-5p (SEQ ID NO:196), miR-331-3p (SEQ ID NO:181), miR-340# (SEQ ID NO:127), miR-362-5p (SEQ ID NO:183), miR-374a (SEQ ID NO:68), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-668 (SEQ ID NO:194), miR-758 (SEQ ID NO:190), and combinations thereof.

In some embodiments, the one, two, three, four, five, six, seven or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2 (SEQ ID NO:78), let-7g# (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), and combinations thereof.

In some embodiments, the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), and combinations thereof.

In some embodiments, the one, two, three, four, five, six, seven or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), and combinations thereof.

In some embodiments, an “expression pattern” based on (a) the presence or increased levels of selected microRNAs, relative to a predetermined criterion or range, combined with (b) the absence or decreased levels of additional selected microRNAs, relative to a predetermined criterion or range (e.g. increased or decreased relative to levels in samples of patients without IPF), is indicative of a diagnosis of IPF.

In any of the embodiments disclosed herein, the method optionally comprises administering a therapeutic agent to the subject. Exemplary therapeutic agents include, but are not limited to, the agents selected from the group consisting of steroids (including but not limited to prednisolone), cytotoxic agents (including but not limited to azathioprine and cyclophosphamide), bardoxolone, LPA agonists (including but not limited to AM152); Torisel (temsirolimus); PI3K inhibitors; pentraxin or serum amyloid P (including but not limited to Pentraxin-2 (PTX-2 or PRM-151)); MEK inhibitors (including but not limited to ARRY-162 and ARRY-300); p38 inhibitors; PAI-1 inhibitors (including but not limited to Tiplaxtinin); agents that reduce the activity of transforming growth factor-beta (TGF-β) (including but not limited to GC-1008 (Genzyme/MedImmune); lerdelimumab (CAT-152; Trabio, Cambridge Antibody); metelimumab (CAT-192, Cambridge Antibody,); LY-2157299 (Eli Lilly); ACU-HTR-028 (Opko Health)) including antibodies that target one or more TGF-β isoforms, inhibitors of TGF-β receptor kinases TGFBR1 (ALK5) and TGFBR2, and modulators of post-receptor signaling pathways; chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610; PS-433540; S-0139; sitaxentan sodium; TBC-3711; zibotentan); agents that reduce the activity of connective tissue growth factor (CTGF) (including but not limited to FG-3019, FibroGen), and including other CTGF-neutralizing antibodies; matrix metalloproteinase (MMP) inhibitors (including but not limited to MMPI-12, PUP-1 and tigapotide triflutate); agents that reduce the activity of epidermal growth factor receptor (EGFR) including but not limed to erlotinib, gefitinib, BMS-690514, cetuximab, antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Ra) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573 or rapamycin); agents that reduce levels of copper such as tetrathiomolybdate; agents that reduce oxidative stress including N-acetyl cysteine and tetrathiomolybdate; and interferon gamma, inhibitors of phosphodiesterase 4 (PDE4) (including but not limited to Roflumilast); inhibitors of phosphodiesterase 5 (PDE5) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton), compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil, P-1894B, and safironil) and peroxisome proliferator-activated receptor (PPAR)-gamma agonists (including but not limited to pioglitazone and rosiglitazone), and combinations thereof.

In any of the embodiments described herein, the therapeutic agent can be an oligonucleotide that decreases the activity or level of expression of one or more of the microRNA in the subject. Alternatively, the therapeutic agent can be an oligonucleotide that increases the activity or level of expression of one or more of the microRNA in the subject.

In any of the embodiments described herein, the blood sample can be selected from the group consisting of whole blood, serum, plasma, exosomes and isolated micro vesicles. In one exemplary embodiment, the blood sample is plasma.

The therapeutic agent can also be an anti-fibrotic agent, such as pirfenidone.

Kits, diagnostic test systems and computer program products are also contemplated.

A kit to be used in the diagnosis of subjects having idiopathic pulmonary fibrosis as described herein (IPF) preferably comprises one or more probes that specifically hybridize to, or primers that specifically amplify, one, two, three, four, five, six, seven or more microRNAs selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a# (SEQ ID NO: 155), miR-106a (SEQ ID NO:156), miR-1227 (SEQ ID NO:157), miR-128 (SEQ ID NO: 158), miR-132 (SEQ ID NO:159), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO: 179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO: 191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), miR-1183 (SEQ ID NO:197), miR-1233 (SEQ ID NO:198), miR-1247 (SEQ ID NO:199), miR-1249 (SEQ ID NO:200), miR-1270 (SEQ ID NO:201), miR-1274A (SEQ ID NO:202), miR-1275 (SEQ ID NO: 203), miR-1298 (SEQ ID NO:204), miR-135b (SEQ ID NO:205), miR-138 (SEQ ID NO: 206), miR-145 (SEQ ID NO:207), miR-15a (SEQ ID NO:208), miR-181c (SEQ ID NO: 209), miR-186 (SEQ ID NO:210), miR-18a# (SEQ ID NO:211), miR-193a-3p (SEQ ID NO:212), miR-199b-5p (SEQ ID NO:213), miR-200a (SEQ ID NO:214), miR-205 (SEQ ID NO:215), miR-20b (SEQ ID NO:216), miR-214 (SEQ ID NO:217), miR-214# (SEQ ID NO:218), miR-218 (SEQ ID NO:219), miR-220b (SEQ ID NO:220), miR-223 (SEQ ID NO:221), miR-23b (SEQ ID NO:222), miR-30a-3p (SEQ ID NO:223), miR-30e-3p (SEQ ID NO:224), miR-326 (SEQ ID NO:225), miR-346 (SEQ ID NO:226), miR-431 (SEQ ID NO:227), miR-450a (SEQ ID NO:228), miR-450b-5p (SEQ ID NO:229), miR-455-5p (SEQ ID NO:230), miR-487a (SEQ ID NO:231), miR-493 (SEQ ID NO:232), miR-494 (SEQ ID NO:233), miR-501-5p (SEQ ID NO:234), miR-505# (SEQ ID NO:235), miR-511 (SEQ ID NO:236), miR-518d-3p (SEQ ID NO:237), miR-518e (SEQ ID NO:238), miR-518f (SEQ ID NO:239), miR-548c-3p (SEQ ID NO:240), miR-570 (SEQ ID NO:241), miR-571 (SEQ ID NO:242), miR-577 (SEQ ID NO:243), miR-618 (SEQ ID NO:244), miR-744# (SEQ ID NO:245), miR-769-5p (SEQ ID NO:246), miR-885-5p (SEQ ID NO: 247), miR-892b (SEQ ID NO:248), miR-9# (SEQ ID NO:249), miR-95 (SEQ ID NO:250), and combinations thereof.

Also provided is a diagnostic test system adapted for performing any of the diagnostic methods described herein. Such a diagnostic test system can comprise means for obtaining test results comprising the activity or level of one or more microRNA correlated with a diagnosis of idiopathic pulmonary fibrosis (IPF) in a blood sample of the subject; means for collecting and tracking test results for one or more individual blood sample; means for comparing the activity or level of one or more microRNA to a predetermined criterion; and means for reporting whether the activity or level of the one or more microRNA meets or exceeds the predetermined criterion.

A computer program product comprising computer-executable instructions embodied in a computer-readable medium for performing the steps of any of the diagnostic methods described herein is also provided.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

### BRIEF DESCRIPTION OF THE FIGURES

FIG. 1 is a Principle Component Analysis (PCA) of IPF and healthy control subjects based on the data provided in Example 1. A principle component analysis is based on a group of differentially regulated miRNAs shows a clear separation of IPF and healthy control subjects. The analysis is based on normalized values (ΔCt) and includes the ten most statistically significantly upregulated (relatively increased) and ten most statistically significantly downregulated (relatively decreased) sequences, as identified by ANOVA.

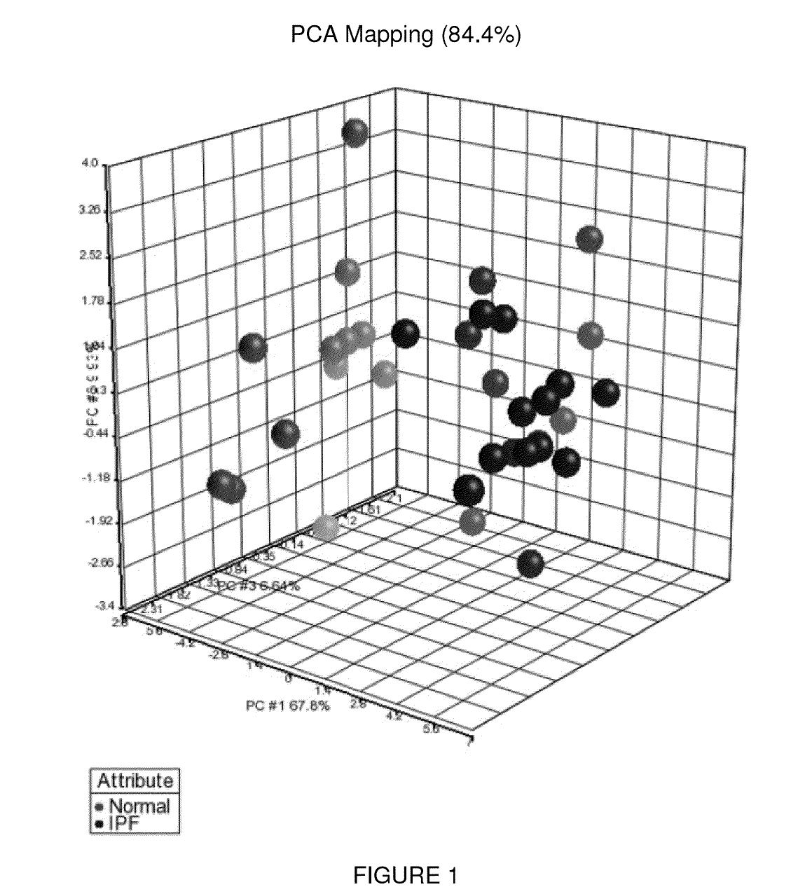

FIG. 2 is another PCA of IPF and healthy control subjects based on the data provided in Example 2. The analysis is based on 86 differentially expressed miRNAs, as identified by ANOVA with FDR≦0.05.

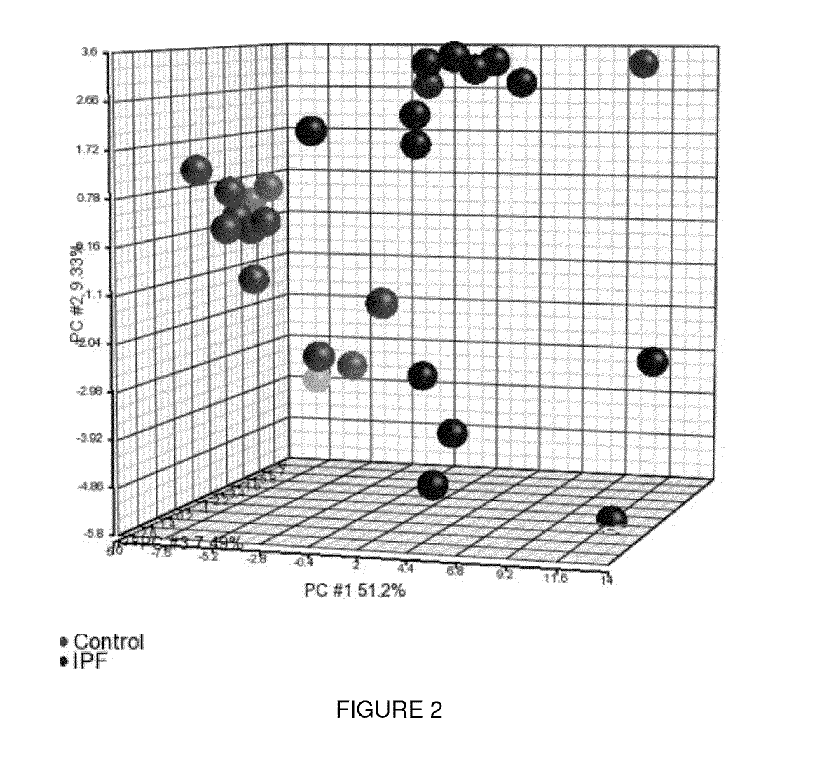

### DETAILED DESCRIPTION OF THE INVENTION

The present application is based on the discovery that the level of one or more microRNAs (including an increased level of, presence of, decreased level of, or absence of such microRNAs) or an alteration in the expression pattern of one or more microRNAs in a blood sample of a human subject is a useful tool for diagnosing the subject with idiopathic pulmonary fibrosis (IPF). The diagnostic methods described herein may permit earlier diagnosis and therapeutic intervention than regimens that rely on conventional clinical diagnosis. The methods described herein also provide a less invasive method of diagnosis compared to conventional methods that utilize a lung tissue sample for diagnosis.

Described herein are methods of diagnosis comprising detecting/measuring a level and/or expression pattern of a disease-associated microRNA (“miRNA”) in a blood sample of a human subject. Detection of a differential blood level or expression pattern of one or more disease-associated miRNAs compared to a control may be used to diagnose a patient suffering from the disease or at risk of suffering from the disease (e.g., idiopathic pulmonary fibrosis), to determine when to begin administering a therapeutic agent, or to select an increased or decreased amount of the therapeutic agent. Expression levels and/or expression patterns of one or more disease-associated miRNAs may also be used to monitor the treatment and disease state of a patient. Such methods include administering a therapeutic agent to a patient, and detecting levels and/or expression patterns of one or more disease-associated miRNAs at periodic intervals, e.g. about one week, one month, two months, three months, or six months. Furthermore, levels of one or more disease-associated miRNAs may allow the screening of drug candidates for altering a particular miRNA profile associated with disease.

In any of the diagnostic methods, diagnostic devices/apparatus, or treatment methods described herein, the selection of miRNAs screened may include, or exclude, one, two, three, four, five, six, more, or all of miR-128 (SEQ ID NO:158), miR-132 (SEQ ID NO: 159), miR-150 (SEQ ID NO:27), miR-21 (SEQ ID NO:51), miR-324-3p (SEQ ID NO:107), let-7d (SEQ ID NO:45), miR-103 (SEQ ID NO:105), miR-125a-5p (SEQ ID NO:176), miR-142-3p (SEQ ID NO:38), miR-26a (SEQ ID NO:70), miR-29b (SEQ ID NO:125), miR-30b (SEQ ID NO:42), miR-30c (SEQ ID NO:52) and miR-362 (SEQ ID NO:96).

In any of the diagnostic methods, diagnostic devices/apparatus, or treatment methods herein, the selection of miRNAs screened may exclude one, two, three, four, five, six, more, or all of miR-21 (SEQ ID NO:51), miR-17 (SEQ ID NO:162), let-7a (SEQ ID NO:173), miR-106a (SEQ ID NO:156), miR-222 (SEQ ID NO:16), miR-146a (SEQ ID NO:21), miR-132 (SEQ ID NO:159), miR-142-3p (SEQ ID NO:38), let-7f (SEQ ID NO: 174), miR-128 (SEQ ID NO:158), miR-150 (SEQ ID NO:27), miR-152 (SEQ ID NO:130), miR-103 (SEQ ID NO:105), miR-26a (SEQ ID NO:70), miR-99b (SEQ ID NO:122), miR-107 (SEQ ID NO:175), miR-122 ((SEQ ID NO:22), miR-141 (SEQ ID NO:161), miR-200c (SEQ ID NO:179) and miR-130a (SEQ ID NO:112).

In one aspect, described herein are methods of diagnosing idiopathic pulmonary fibrosis (IPF) in a human subject comprising detecting or measuring in a blood sample from the subject the level or expression pattern of one or more microRNAs, wherein an alteration in the level or expression pattern of the one or more microRNA relative to a predetermined criterion is indicative of idiopathic pulmonary fibrosis in the subject. For some microRNAs, a higher level relative to a predetermined criterion is indicative of IPF, while for other microRNAs, a lower level relative to a predetermined criterion is indicative of IPF. Such microRNAs which exhibit differential expression in IPF patients, compared to the expression pattern of the same microRNAs in control patients without IPF, are termed “disease-associated miRNAs or IPF-associated miRNAs.” The methods may further comprise the step of comparing the level or expression pattern of the one or more microRNAs to the predetermined criterion. In related embodiments, the method of diagnosing comprises detecting a level of one or more microRNAs that falls within a predetermined range indicative of IPF. This predetermined range of levels is typically different from (higher or lower than) the levels of the respective microRNAs seen in patients without IPF.

The term “differential expression” as used herein refers to both quantitative as well as qualitative differences in the expression patterns of one or more microRNAs in a blood sample versus the expression patterns of the one or more microRNAs in a blood sample from a healthy subject. For example, a differentially expressed microRNA may either be present or absent in normal versus disease conditions, or may be increased or decreased in a disease condition versus a# normal condition. Such a qualitatively regulated microRNA may exhibit an expression pattern within a blood sample that is detectable in either control or disease conditions, but is not detectable in both. In other words, a microRNA is differentially expressed when expression of the microRNA occurs at a different level (higher or lower, presence or absence) in the blood sample of a subject with IPF relative to the level of its expression in the blood sample from a disease-free subject without IPF. The level of a differentially expressed microRNA may refer to either the uncorrected (raw) or normalized abundance of a microRNA in a sample. Comparisons of microRNA levels may consider the uncorrected quantified abundance of a given microRNA relative to an uncorrected reference value. Alternatively, the abundance of a given microRNA may be expressed as a ratio relative to one or more additional microRNAs (or other internal controls) in that sample. In such a case, this “normalized” ratio would be compared relative to a similar “normalized” reference value from a sample of healthy patients (or patients without IPF).

As used herein the terms “microRNA,” “miR,” “mir,” and “miRNA” are used to refer to a class of small RNA molecules that are capable of modulating RNA levels (see, Zeng and Cullen, RNA, 9(1): 112-123, 2003; Kidner and Martienssen Trends Genet, 19(1): 13-6, 2003; Dennis C, Nature, 420(6917):732, 2002; Couzin J, Science 298(5602):2296-7, 2002, each of which is incorporated by reference herein). microRNAs are a class of small non-coding RNAs that generally function as post-transcriptional gene regulators. In some cases, microRNAs can hybridize to the 3′ untranslated region (UTR) of RNAs, often mRNAs, and can mediate translational repression or RNA cleavage/destruction. Recent studies have shown that microRNAs provide important regulatory functions in a variety of biological processes including cell proliferation, differentiation, development, and apoptosis.

Without being bound to theory, a gene coding for a miRNA may be transcribed leading to production of an miRNA precursor known as the “pri-miRNA”. The pri-miRNA may be part of a polycistronic RNA comprising multiple pri-miRNAs. The pri-miRNA may form a hairpin with a stem and loop, and the stem may comprise mismatched bases.

The hairpin structure of the pri-miRNA may be recognized by Drosha, which is an RNase III endonuclease. Processing by Drosha may yield a pre-miRNA stem loop having a 5′ phosphate and about a 2 nucleotide 3′ overhang. The details of pri-miRNA processing are well known in the art, and may be found, e.g., in U.S. Pat. Publication No. 20070050146, which is incorporated herein by reference in its entirety.

A subject having or at risk for developing IPF will exhibit altered levels or expression pattern of certain miRNAs (increased or decreased relative to a predetermined criterion specific to the miRNA, or falling within a predetermined range that is correlated with IPF or falling outside of a predetermined range that is correlated with patients that do not have IPF, e.g. healthy patients). In any of the embodiments described herein, the expression pattern, presence or an increased level of one or more microRNAs is detected, relative to a predetermined criterion, and is indicative of a diagnosis of IPF, and such one, two, three, four, five, six, seven or more microRNAs comprises a nucleotide sequence selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), let-7b (SEQ ID NO:76), miR-130a (SEQ ID NO:112), miR-142-5p (SEQ ID NO:43), miR-148a (SEQ ID NO:91), miR-152 (SEQ ID NO:130), miR-15a# (SEQ ID NO:64), miR-19a (SEQ ID NO:74), miR-21 (SEQ ID NO:51), miR-21# (SEQ ID NO:141), miR-26a-2# (SEQ ID NO:82), miR-20a (SEQ ID NO:89), miR-324-3p (SEQ ID NO:107), miR-335 (SEQ ID NO:119), miR-362-3p (SEQ ID NO:96, miR-590-5p (SEQ ID NO:104), miR-598 (SEQ ID NO:110), let-7a# (SEQ ID NO:155), miR-106a (SEQ ID NO: 156), miR-1227 (SEQ ID NO:157), miR-128 (SEQ ID NO:158), miR-132 (SEQ ID NO: 159), miR-140-5p (SEQ ID NO:160), miR-141 (SEQ ID NO:161), miR-17 (SEQ ID NO: 162), miR-185 (SEQ ID NO:163), miR-30a-5p (SEQ ID NO:164), miR-30d (SEQ ID NO: 165), miR-34a (SEQ ID NO:166), miR-378 (SEQ ID NO:167), miR-425 (SEQ ID NO: 168), miR-429 (SEQ ID NO:169), miR-579 (SEQ ID NO:170), miR-523 (SEQ ID NO: 171), miR-551b# (SEQ ID NO:172), miR-1183 (SEQ ID NO:197), miR-1233 (SEQ ID NO: 198), miR-1247 (SEQ ID NO:199), miR-1270 (SEQ ID NO:201), miR-1274A (SEQ ID NO:202), miR-1275 (SEQ ID NO:203), miR-135b (SEQ ID NO:205), miR-138 (SEQ ID NO:206), miR-186 (SEQ ID NO:210), miR-193a-3p (SEQ ID NO:212), miR-200a (SEQ ID NO:214), miR-205 (SEQ ID NO:215), miR-20b (SEQ ID NO:216), miR-214 (SEQ ID NO:217), miR-214# (SEQ ID NO:218), miR-218 (SEQ ID NO:219), miR-220b (SEQ ID NO:220), miR-223 (SEQ ID NO:221), miR-30a-3p (SEQ ID NO:223), miR-326 (SEQ ID NO:225), miR-346 (SEQ ID NO:226), miR-450a (SEQ ID NO:228), miR-450b-5p (SEQ ID NO:229), miR-455-5p (SEQ ID NO:230), miR-501-5p (SEQ ID NO:234), miR-511 (SEQ ID NO:236), miR-518d-3p (SEQ ID NO:237), miR-518e (SEQ ID NO:238), miR-518f (SEQ ID NO:239), miR-548c-3p (SEQ ID NO:240), miR-570 (SEQ ID NO:241), miR-571 (SEQ ID NO:242), miR-618 (SEQ ID NO:244), miR-885-5p (SEQ ID NO:247), miR-9# (SEQ ID NO:249), miR-95 (SEQ ID NO:250), or fully complementary nucleotide sequences thereto and combinations thereof, microRNAs that comprise at least 15, 16, 17, 18, 19, 20, 21, 22, 23, 24, 25 or more consecutive bases of any of these SEQ ID NOs or fully complementary nucleotide sequences thereto, or combinations thereof; microRNAs that are at least 80% or 85% or 90% or 95% or more identical to the nucleotide sequence of any of these SEQ ID NOs or fully complementary nucleotide sequences thereto, or combinations thereof; or a nucleotide sequence that hybridizes to any of these SEQ ID NOs or the full complement thereof, or combinations thereof. In some embodiments, the one, two, three, four, five, six, seven or more microRNAs comprises a nucleotide sequence selected from the group consisting of miR-222 (SEQ ID NO:16), miR-345 (SEQ ID NO:18), miR-146a (SEQ ID NO:21), miR-122 (SEQ ID NO:22), miR-146b-5p (SEQ ID NO:24), miR-1300 (SEQ ID NO:25), miR-150 (SEQ ID NO:27), miR-130a (SEQ ID NO:112), miR-142-5p (SEQ ID NO:43), miR-148a (SEQ ID NO:91), miR-152 (SEQ ID NO:130), miR-15a# (SEQ ID NO: 64), miR-19a (SEQ ID NO:74), miR-21 (SEQ ID NO:51), miR-21# (SEQ ID NO:141), miR-26a-2# (SEQ ID NO:82), miR-324-3p (SEQ ID NO:107), miR-335 (SEQ ID NO:119), miR-362-3p (SEQ ID NO:96), miR-590-5p (SEQ ID NO:104), miR-598 (SEQ ID NO:110), let-7a# (SEQ ID NO:155), miR-106a (SEQ ID NO:156), miR-1227 (SEQ ID NO:157), miR-128 (SEQ ID NO:158), miR-132 (SEQ ID NO:159), miR-140-5p (SEQ ID NO:160), miR-141 (SEQ ID NO:161), miR-17 (SEQ ID NO:162), miR-185 (SEQ ID NO:163), miR-30a-5p (SEQ ID NO:164), miR-30d (SEQ ID NO:165), miR-34a (SEQ ID NO:166), miR-378 (SEQ ID NO:167), miR-425 (SEQ ID NO:168), miR-429 (SEQ ID NO:169), miR-579 (SEQ ID NO:170), miR-523 (SEQ ID NO:171), miR-551b# (SEQ ID NO:172), miR-1183 (SEQ ID NO:197), miR-1233 (SEQ ID NO:198), miR-1247 (SEQ ID NO:199), miR-1270 (SEQ ID NO:201), miR-1274A (SEQ ID NO:202), miR-1275 (SEQ ID NO:203), miR-135b (SEQ ID NO:205), miR-138 (SEQ ID NO:206), miR-186 (SEQ ID NO:210), miR-193a-3p (SEQ ID NO:212), miR-200a (SEQ ID NO:214), miR-205 (SEQ ID NO:215), miR-20b (SEQ ID NO:216), miR-214 (SEQ ID NO:217), miR-214# (SEQ ID NO:218), miR-218 (SEQ ID NO:219), miR-220b (SEQ ID NO:220), miR-223 (SEQ ID NO:221), miR-30a-3p (SEQ ID NO:223), miR-326 (SEQ ID NO:225), miR-346 (SEQ ID NO:226), miR-450a (SEQ ID NO:228), miR-450b-5p (SEQ ID NO:229), miR-455-5p (SEQ ID NO: 230), miR-501-5p (SEQ ID NO:234), miR-511 (SEQ ID NO:236), miR-518d-3p (SEQ ID NO:237), miR-518e (SEQ ID NO:238), miR-518f (SEQ ID NO:239), miR-548c-3p (SEQ ID NO:240), miR-570 (SEQ ID NO:241), miR-571 (SEQ ID NO:242), miR-618 (SEQ ID NO:244), miR-885-5p (SEQ ID NO:247), miR-9# (SEQ ID NO:249), miR-95 (SEQ ID NO:250), or fully complementary nucleotide sequences thereto, or combinations thereof; microRNAs that comprise at least 15, 16, 17, 18, 19, 20, 21, 22, 23, 24, 25 or more consecutive bases of any of these SEQ ID NOs or fully complementary nucleotide sequences thereto, or combinations thereof; microRNAs that are at least 80% or 85% or 90% or 95% or more identical to the nucleotide sequence of any of these SEQ ID NOs or fully complementary nucleotide sequences thereto, or combinations thereof; or a nucleotide sequence that hybridizes to any of these SEQ ID NOs or the full complement thereof, or combinations thereof.

Alternatively, the level of one, two, three, four, five, six, seven or more microRNAs is detected as falling within a predetermined range that is correlated with IPF. This predetermined range of levels is typically higher than the levels seen in patients without IPF.

In any of the embodiments described herein, the absence or a decreased level of one, two, three, four, five, six, seven or more microRNAs is detected, relative to a predetermined criterion, and is indicative of a diagnosis of IPF, and such one or more microRNAs comprises a nucleotide sequence selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-20a# (SEQ ID NO:75), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-28-5p (SEQ ID NO:90), miR-106b# (SEQ ID NO:92), let-7e (SEQ ID NO:93), miR-25 (SEQ ID NO:94), miR-656 (SEQ ID NO:95), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-103 (SEQ ID NO:105), miR-19b (SEQ ID NO:106), miR-145 (SEQ ID NO:108), miR-199a-3p (SEQ ID NO:109), -151-5P (SEQ ID NO:111), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), miR-1249 (SEQ ID NO:200), miR-145 (SEQ ID NO:207), miR-15a (SEQ ID NO:208), miR-181c (SEQ ID NO:209), miR-18a# (SEQ ID NO:211), miR-199b-5p (SEQ ID NO:213), miR-23b (SEQ ID NO:222), miR-30e-3p (SEQ ID NO:224), miR-431 (SEQ ID NO:227), miR-487a (SEQ ID NO:231), miR-493 (SEQ ID NO:232), miR-494 (SEQ ID NO:233), miR-505# (SEQ ID NO:235), or fully complementary nucleotide sequences thereto, or combinations thereof; microRNAs that comprise at least 15, 16, 17, 18, 19, 20, 21, 22, 23, 24, 25 or more consecutive bases of any of these SEQ ID NOs or fully complementary nucleotide sequences thereto, or combinations thereof; microRNAs that are at least 80% or 85% or 90% or 95% or more identical to the nucleotide sequence of any of these SEQ ID NOs or fully complementary nucleotide sequences thereto, or combinations thereof; or a nucleotide sequence that hybridizes to any of these SEQ ID NOs or the full complement thereof, or combinations thereof. In some embodiments, the one, two, three, four, five, six, seven or more microRNAs comprises a nucleotide sequence selected from the group consisting of miR-1244 (SEQ ID NO:20), miR-142-3p (SEQ ID NO:38), miR-30b (SEQ ID NO:42), let-7d (SEQ ID NO:45), miR-30c (SEQ ID NO:52), miR-15b (SEQ ID NO:56), miR-190 (SEQ ID NO:63), miR-301a (SEQ ID NO:67), miR-374a (SEQ ID NO:68), miR-144# (SEQ ID NO:69), miR-26a (SEQ ID NO:70), let-7g (SEQ ID NO:72), miR-20a# (SEQ ID NO:75), miR-28-5p (SEQ ID NO:90), let-7e (SEQ ID NO:93), miR-127-3p (SEQ ID NO:101), miR-103 (SEQ ID NO:105), miR-24-2# (SEQ ID NO:120), miR-99b (SEQ ID NO:122), miR-411 (SEQ ID NO:124), miR-29b (SEQ ID NO:125), miR-340# (SEQ ID NO:127), miR-148b# (SEQ ID NO:128), miR-543 (SEQ ID NO:133), miR-154# (SEQ ID NO:139), miR-196b (SEQ ID NO:140), miR-411# (SEQ ID NO:147), miR-548J (SEQ ID NO:148), miR-664 (SEQ ID NO:152), let-7a (SEQ ID NO:173), let-7f (SEQ ID NO:174), miR-107 (SEQ ID NO:175), miR-125a-5p (SEQ ID NO:176), miR-181a-2# (SEQ ID NO: 177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), miR-1249 (SEQ ID NO:200), miR-145 (SEQ ID NO:207), miR-15a (SEQ ID NO:208), miR-181c (SEQ ID NO:209), miR-18a# (SEQ ID NO:211), miR-199b-5p (SEQ ID NO:213), miR-23b (SEQ ID NO:222), miR-30e-3p (SEQ ID NO:224), miR-431 (SEQ ID NO:227), miR-487a (SEQ ID NO:231), miR-493 (SEQ ID NO:232), miR-494 (SEQ ID NO:233), miR-505# (SEQ ID NO:235), or fully complementary nucleotide sequences thereto, or combinations thereof; microRNAs that comprise at least 15, 16, 17, 18, 19, 20, 21, 22, 23, 24, 25 or more consecutive bases of any of these SEQ ID NOs or fully complementary nucleotide sequences thereto, or combinations thereof; microRNAs that are at least 80% or 85% or 90% or 95% or more identical to the nucleotide sequence of any of these SEQ ID NOs or fully complementary nucleotide sequences thereto, or combinations thereof; or a nucleotide sequence that hybridizes to any of these SEQ ID NOs or the full complement thereof, or combinations thereof.

In embodiments where samples are collected as described in Example 1, the expression pattern, absence or a decreased level of one or more microRNAs is detected, relative to the control, is indicative of a diagnosis of IPF, and such one, two, three, four, five, six, seven or more microRNAs comprises a nucleotide sequence selected from the group consisting of let-7b (SEQ ID NO:76), miR-148a (SEQ ID NO:91), miR-130a (SEQ ID NO: 112), miR-152 (SEQ ID NO:130), miR-142-5p (SEQ ID NO:43), miR-15a# (SEQ ID NO: 64), miR-19a (SEQ ID NO:74), miR-21 (SEQ ID NO:51), miR-324-3p (SEQ ID NO:107), miR-335 (SEQ ID NO:119), miR-362-3p (SEQ ID NO:96), miR-590-5p (SEQ ID NO:104), miR-20a (SEQ ID NO:89), miR-598 (SEQ ID NO:110), or fully complementary nucleotide sequences thereto, or combinations thereof; microRNAs that comprise at least 15, 16, 17, 18, 19, 20, 21, 22, 23, 24, 25 or more consecutive bases of any of these SEQ ID NOs or fully complementary nucleotide sequences thereto, or combinations thereof; microRNAs that are at least 80% or 85% or 90% or 95% or more identical to the nucleotide sequence of any of these SEQ ID NOs or fully complementary nucleotide sequences thereto, or combinations thereof; or a nucleotide sequence that hybridizes to any of these SEQ ID NOs or the full complement thereof, or combinations thereof.

Any of these preceding nucleotide sequences that are differentially expressed (i.e. increased or decreased levels are observed in samples from patients with IPF, compared to levels in samples from patients without IPF) are encompassed by the term “disease-associated miRNA” or “IPF-associated miRNA.”

The terms “identical” or “percent identity,” in the context of two or more polynucleotide or polypeptide sequences, refer to two or more sequences or subsequences that are the same or have a specified percentage of nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm calculates the percent sequence identity for the test sequence(s) relative to the reference sequence, based on the designated program parameters.

Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575Science Dr., Madison, Wis.), or by visual inspection.

Alternatively, the level of such one or more microRNAs is detected as falling within a predetermined range that is correlated with IPF. This predetermined range of levels is typically lower than the levels seen in patients without IPF.

In any of the embodiments described herein, the level of multiple different microRNAs is detected or measured. Thus, for example, the preceding methods of diagnosing idiopathic pulmonary fibrosis (IPF) in a human subject may comprise (a) detecting or measuring in a blood sample from the subject the level of a first microRNA, wherein an alteration in the level of the first microRNA relative to a first predetermined criterion is indicative of IPF (or wherein detection of a level falling within a first predetermined range is indicative of IPF), and (b) detecting or measuring in a blood sample from the subject the level of a second microRNA, wherein an alteration in the level of the second microRNA relative to a second predetermined criterion is indicative of IPF (or wherein detection of a level falling within a second predetermined range is indicative of IPF); and optionally further comprising the steps of (c) comparing the level of the first microRNA to the first predetermined criterion or range and (d) comparing the level of the second microRNA to the second predetermined criterion or range. Similarly, the methods may further comprise detecting or measuring the level of a third microRNA and optionally comparing the level of the third microRNA to a third predetermined criterion or range. Steps may be repeated for fourth, fifth, sixth or more microRNAs. For example, 1, 2, 3, 4, 5, 6, 7, 8, 9, 10, 11, 12, 13, 14, 15, 16, 17, 18, 19, 20, 25, 30, 35, 40 or more different microRNAs can be detected and can be compared to the respective predetermined criterion or range for the microRNA. It is understood that combinations of one or more of the microRNAs herein include any possible combination of any of the nucleotide sequences described herein, without having to list every combination.

The term “predetermined criterion” as used herein refers to a number indicative of the level of microRNA obtained from prior measurements of the microRNA in blood samples from a plurality of subjects without IPF. In some variations, the predetermined criterion is the level of microRNA in healthy human controls (i.e., subjects with no clinical manifestation of any respiratory disorder), in which case the level of one, two, three, four, five, six, seven or more microRNAs in idiopathic pulmonary fibrosis is either increased (e.g., miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-130a (SEQ ID NO:112), miR-142-5p (SEQ ID NO:43), miR-148a (SEQ ID NO:91), miR-152 (SEQ ID NO:130), miR-15a# (SEQ ID NO:64), miR-19a (SEQ ID NO:74), let-7b (SEQ ID NO:76), miR-21 (SEQ ID NO:51), miR-20a (SEQ ID NO:89), miR-21# (SEQ ID NO:141), miR-26a-2# (SEQ ID NO:82), miR-324-3p (SEQ ID NO:107), miR-335 (SEQ ID NO:119), miR-362-3p (SEQ ID NO:96), miR-590-5p (SEQ ID NO:104), miR-598 (SEQ ID NO:110), let-7a# (SEQ ID NO: 155), miR-106a (SEQ ID NO:156), miR-1227 (SEQ ID NO:157), miR-128 (SEQ ID NO: 158), miR-132 (SEQ ID NO:159), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172, miR-1183 (SEQ ID NO: 197), miR-1233 (SEQ ID NO:198), miR-1247 (SEQ ID NO:199), miR-1270 (SEQ ID NO: 201), miR-1274A (SEQ ID NO:202), miR-1275 (SEQ ID NO:203), miR-135b (SEQ ID NO:205), miR-138 (SEQ ID NO:206), miR-186 (SEQ ID NO:210), miR-193a-3p (SEQ ID NO:212), miR-200a (SEQ ID NO:214), miR-205 (SEQ ID NO:215), miR-20b (SEQ ID NO:216), miR-214 (SEQ ID NO:217), miR-214# (SEQ ID NO:218), miR-218 (SEQ ID NO:219), miR-220b (SEQ ID NO:220), miR-223 (SEQ ID NO:221), miR-30a-3p (SEQ ID NO:223), miR-326 (SEQ ID NO:225), miR-346 (SEQ ID NO:226), miR-450a (SEQ ID NO:228), miR-450b-5p (SEQ ID NO:229), miR-455-5p (SEQ ID NO:230), miR-501-5p (SEQ ID NO:234), miR-511 (SEQ ID NO:236), miR-518d-3p (SEQ ID NO:237), miR-518e (SEQ ID NO:238), miR-518f (SEQ ID NO:239), miR-548c-3p (SEQ ID NO:240), miR-570 (SEQ ID NO:241), miR-571 (SEQ ID NO:242), miR-618 (SEQ ID NO:244), miR-885-5p (SEQ ID NO:247), miR-9# (SEQ ID NO:249), miR-95 (SEQ ID NO:250)) or decreased (e.g., miR-1244 (SEQ ID NO:20), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-20a# (SEQ ID NO:75), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-28-5p (SEQ ID NO:90), miR-106b# (SEQ ID NO:92), let-7e (SEQ ID NO:93), miR-25 (SEQ ID NO:94), miR-656 (SEQ ID NO:95), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-103 (SEQ ID NO:105), miR-19b (SEQ ID NO:106), miR-145# (SEQ ID NO:108), miR-199a-3p (SEQ ID NO:109), miR-151-5P (SEQ ID NO:111), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a (SEQ ID NO: 173), let-7f (SEQ ID NO:174), miR-107 (SEQ ID NO:175), miR-125a-5p (SEQ ID NO: 176), miR-181a-2# (SEQ ID NO:177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO: 194), miR-1256 (SEQ ID NO:195), miR-299-5p (SEQ ID NO:196, miR-1249 (SEQ ID NO: 200), miR-145 (SEQ ID NO:207), miR-15a (SEQ ID NO:208), miR-181c (SEQ ID NO: 209), miR-18a# (SEQ ID NO:211), miR-199b-5p (SEQ ID NO:213), miR-23b (SEQ ID NO:222), miR-30e-3p (SEQ ID NO:224), miR-431 (SEQ ID NO:227), miR-487a (SEQ ID NO:231), miR-493 (SEQ ID NO:232), miR-494 (SEQ ID NO:233), miR-505# (SEQ ID NO:235), compared to the level or expression pattern of microRNA in a blood sample obtained from the healthy controls.

The term “predetermined range” as used herein refers to a range of levels or measurements of microRNA typically observed in human IPF subjects, in which case the level of the microRNA is indicative of IPF if it falls within the predetermined range.

In other variations, the predetermined criterion or range might include information such as mean, standard deviation, quartile measurements, confidence intervals, or other information about the distribution or range of microRNA concentration in IPF subjects or subjects without IPF. In still other variations, the predetermined criterion is a receiver operating characteristic curve based on data of microRNA measurements in subjects with IPF and subjects that do not have IPF. In still other variations, the predetermined criterion is a cutoff value of microRNA measurements, wherein the cutoff value is determined, based on previous measurements to discriminate IPF with a sensitivity and specificity calculated from measurements of microRNA in human subjects with IPF and non-IPF human subjects. Optionally, the predetermined criterion is based on subjects further stratified by other characteristics that can be determined for a subject, to further refine the diagnostic precision. Such additional characteristics include, for example, sex, age, weight, smoking habits, race or ethnicity, blood pressure, other diseases, and medications.

The “level” of a nucleic acid (i.e., microRNA) in the methods described herein is the amount of the nucleic acid or its activity as measured by standard laboratory methods. The term includes the amount of nucleic acid (e.g., concentration or total amount) detected in a sample, e.g., by northern blot or microarray analysis or quantitative RT-PCR methods, as well as detection of the presence or absence of the nucleic acid. In one embodiment, the level of a disease-associated miRNA is measured using an amplification method such as quantitative real-time PCR (Q-PCR). Such amplification methods will use primers complementary to at least 12 bases of (a) the miRNAs of any of SEQ ID NOS: 1-250 or (b) the full complement thereof. In another embodiment, the level is measured by contacting a biological sample with a probe or biochip and measuring the amount of hybridization. The level of a differentially expressed microRNA may refer to either the uncorrected (raw) or normalized abundance of a microRNA in a sample. Comparisons of microRNA levels may consider the uncorrected quantified abundance of a given microRNA relative to an uncorrected reference value. Alternatively, the abundance of a given microRNA may be expressed as a ratio relative to one or more additional microRNAs (or other internal controls) in that sample. In such a case, this “normalized” ratio would be compared relative to a similar “normalized” reference value from a sample of healthy patients (or patients without IPF).

In a related embodiment, the nucleic acid may be detected by contacting a sample comprising the nucleic acid with a biochip comprising an attached oligonucleotide probe sufficiently complementary to the nucleic acid and detecting hybridization to the probe above control levels. Hybridization of the specific oligonucleotide probes may be detected using Northern Blot analysis, slot-blot analysis or in situ hybridization analysis and any other methods known in the art, such as those techniques described in Sambrook et al. (Molecular Cloning: A Laboratory Manual, Cold Springs Harbor Laboratories (New York, 1989). Hybridization means contacting two or more nucleic acids under conditions suitable for base pairing. Hybridization includes interaction between partially or perfectly complementary nucleic acids. Suitable hybridization conditions are well known to those of skill in the art. In certain applications, it is appreciated that lower stringency conditions may be required. Under these conditions, hybridization may occur even though the sequences of the interacting strands are not perfectly complementary, being mismatched at one or more positions. Conditions may be rendered less stringent by adjusting conditions in accordance with the knowledge in the art, e.g., increasing salt concentration and/or decreasing temperature. Suitable hybridization conditions are those conditions that allow the detection of gene expression from identifiable expression units such as genes. Preferred hybridization conditions are stringent hybridization conditions, such as hybridization at 42° C. in a solution (i.e., a hybridization solution) comprising 50% formamide, 1% SDS, 1 M NaCl, 10% dextran sulfate, and washing for 30 minutes at 65° C. in a wash solution comprising 1×SSC and 0.1% SDS. It is understood in the art that conditions of equivalent stringency can be achieved through variation of temperature and buffer, or salt concentration, as described in Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (2d. Ed.; 1989), pp. 9.47 to 9.51.

The oligonucleotide probes may be labeled for detection of hybridization with the RNA extracted from the biological sample, or the RNA may be labeled for detection. Labels include a radioactive label such as ³H, ¹⁴C, ³²P, ³⁵S, or ¹²⁵I. In addition, the labels may be a fluorescent or chemiluminescent compound, such as fluorescein isothiocyanate, phycoerythrin, rhodamine, or luciferin. The labels may be enzymes such as alkaline phosphatase, β-galactosidase, biotin and avidin or horseradish peroxidase (Bayer et al., Meth. Enz., 184:138-163 (1990)). The oligonucleotide probes may be attached to solid substrates such as membranes, beads, filters, glass, silicon, metal, metal-alloy, anopore, polymeric, nylon or plastic. The substrates may be chemically treated with chemical prior to attaching probes to enhance binding or to inhibit nonspecific binding during use. Exemplary treatments include coating glass slides with coating of aminoalkyl silanes or polymeric materials such as acrylamide or proteins. The probes may be covalently or non-covalently attached to the substrate. Probes or primers may be, e.g., 8-20, 8-30, 8-40, 12-20, 12-30 or 12-40 bases in length.

As used herein, the term “target” as used in the context of an miRNA target refers to the RNA which contains a binding site for the miRNA and which is presumably regulated by the miRNA.

In some embodiments, target gene sequences for an miRNA sequence are determined by comparing the sequence of potential target gene sequences with the miRNA sequence for complementary matches (e.g., for Watson-Crick complementarity pairing and/or G:U pairing). For example, the UTR of potential target gene sequences can be compared with the miRNA sequence for complementary matches, and used to identify those gene sequences with higher degrees of complementarity as being target gene sequences. The determination may be performed manually, or with the aid of a machine such as a computer system.

The expression of or activity of an miRNA described herein can be modulated (increased or decreased) by administering (a) a nucleotide sequence of any of SEQ ID NOS: 1-250, or the full complement thereof, (b) a nucleotide sequence comprising (i) at least 8, 9, 10, 11, 12, 13, 14, or 15 consecutive bases of any of SEQ ID NOS: 1-250, or (ii) the full complement of the at least 8, 9, 10, 11, 12, 13, 14, or 15 consecutive bases; or (c) a nucleotide sequence that is at least 80%, 90% or 95% or more identical to any of SEQ ID NOS: 1-250, or the full complement thereof, or (d) a nucleotide sequence that hybridizes under stringent conditions to any of SEQ ID NOS: 1-250, or the full complement thereof.

One of ordinary skill in the art will appreciate that a complementary sequence need not be an exact complement, and that it is within the scope of the present invention to employ miRNA fragments, fragments of complement sequences, or sequences which are similar to the miRNA or its complement. As one example, the level or activity of an miRNA that is increased in IPF patients, e.g. miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), let-7b (SEQ ID NO:76), miR-130a (SEQ ID NO:112), miR-142-5p (SEQ ID NO:43), miR-148a (SEQ ID NO:91), miR-152 (SEQ ID NO:130), miR-15a# (SEQ ID NO:64), miR-19a (SEQ ID NO:74), miR-21 (SEQ ID NO:51), miR-21# (SEQ ID NO:141), miR-26a-2# (SEQ ID NO:82), miR-20a (SEQ ID NO:89), miR-324-3p (SEQ ID NO:107), miR-335 (SEQ ID NO:119), miR-362-3p (SEQ ID NO:96), miR-590-5p (SEQ ID NO:104), miR-598 (SEQ ID NO:110), let-7a# (SEQ ID NO:155), miR-106a (SEQ ID NO: 156), miR-1227 (SEQ ID NO:157), miR-128 (SEQ ID NO:158), miR-132 (SEQ ID NO: 159), miR-140-5p (SEQ ID NO:160), miR-141 (SEQ ID NO:161), miR-17 (SEQ ID NO: 162), miR-185 (SEQ ID NO:163), miR-30a-5p (SEQ ID NO:164), miR-30d (SEQ ID NO: 165), miR-34a (SEQ ID NO:166), miR-378 (SEQ ID NO:167), miR-425 (SEQ ID NO: 168), miR-429 (SEQ ID NO:169), miR-579 (SEQ ID NO:170), miR-523 (SEQ ID NO: 171), miR-551b# (SEQ ID NO:172), miR-1183 (SEQ ID NO:197), miR-1233 (SEQ ID NO: 198), miR-1247 (SEQ ID NO:199), miR-1270 (SEQ ID NO:201), miR-1274A (SEQ ID NO:202), miR-1275 (SEQ ID NO:203), miR-135b (SEQ ID NO:205), miR-138 (SEQ ID NO:206), miR-186 (SEQ ID NO:210), miR-193a-3p (SEQ ID NO:212), miR-200a (SEQ ID NO:214), miR-205 (SEQ ID NO:215), miR-20b (SEQ ID NO:216), miR-214 (SEQ ID NO:217), miR-214# (SEQ ID NO:218), miR-218 (SEQ ID NO:219), miR-220b (SEQ ID NO:220), miR-223 (SEQ ID NO:221), miR-30a-3p (SEQ ID NO:223), miR-326 (SEQ ID NO:225), miR-346 (SEQ ID NO:226), miR-450a (SEQ ID NO:228), miR-450b-5p (SEQ ID NO:229), miR-455-5p (SEQ ID NO:230), miR-501-5p (SEQ ID NO:234), miR-511 (SEQ ID NO:236), miR-518d-3p (SEQ ID NO:237), miR-518e (SEQ ID NO:238), miR-518f (SEQ ID NO:239), miR-548c-3p (SEQ ID NO:240), miR-570 (SEQ ID NO:241), miR-571 (SEQ ID NO:242), miR-618 (SEQ ID NO:244), miR-885-5p (SEQ ID NO:247), miR-9# (SEQ ID NO:249), miR-95 (SEQ ID NO:250), may be decreased using a sequence which is complementary to the microRNA, a fragment that is complementary to at least 8, 9, 10, 11, 12, 13, 14, or 15 consecutive bases, or, e.g., a sequence which is 80%, 85%, 90%, or 95% or more identical to the complementary sequence (or fragment thereof). In another example, the level or activity of an miRNA that is decreased in IPF patients, e.g., miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-20a# (SEQ ID NO:75), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-28-5p (SEQ ID NO:90), miR-106b# (SEQ ID NO:92), let-7e (SEQ ID NO:93), miR-25 (SEQ ID NO:94), miR-656 (SEQ ID NO:95), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-103 (SEQ ID NO:105), miR-19b (SEQ ID NO:106), miR-145# (SEQ ID NO:108), miR-199a-3p (SEQ ID NO:109), miR-151-5P (SEQ ID NO:111), miR-502-3p (SEQ ID NO:113), miR-136# (SEQ ID NO:114), miR-194 (SEQ ID NO:115), miR-221 (SEQ ID NO:116), miR-22*(SEQ ID NO:117), miR-93 (SEQ ID NO:118), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a (SEQ ID NO: 173), let-7f (SEQ ID NO:174), miR-107 (SEQ ID NO:175), miR-125a-5p (SEQ ID NO: 176), miR-181a-2# (SEQ ID NO:177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO: 194), miR-1256 (SEQ ID NO:195), miR-299-5p (SEQ ID NO:196, miR-1249 (SEQ ID NO: 200), miR-145 (SEQ ID NO:207), miR-15a (SEQ ID NO:208), miR-181c (SEQ ID NO: 209), miR-18a# (SEQ ID NO:211), miR-199b-5p (SEQ ID NO:213), miR-23b (SEQ ID NO:222), miR-30e-3p (SEQ ID NO:224), miR-431 (SEQ ID NO:227), miR-487a (SEQ ID NO:231), miR-493 (SEQ ID NO:232), miR-494 (SEQ ID NO:233), miR-505# (SEQ ID NO:235)), may be increased using the nucleotide sequence comprising the nucleotide sequence of that SEQ ID, a fragment of the nucleotide sequence comprising the sequence of that SEQ ID, or a sequence which is 80%, 85%, 90%, or 95% or more identical to the either the sequence or a fragment thereof.

The potential target gene sequences and miRNA sequences are preferably human.

As noted above, the miRNA may be detected by immobilizing RNA from the blood sample on a solid support such as nylon membranes and hybridizing a labeled probe with the sample. Similarly, the miRNA may also be detected by immobilizing the labeled probe to the solid support and hybridizing the blood sample comprising the miRNA to the probe. Following washing to remove the non-specific hybridization, the label may be detected.

These assays can be direct hybridization assays or can include the use of multiple probes, as is generally outlined in U.S. Pat. Nos. 5,681,702; 5,597,909; 5,545,730; 5,594,117; 5,591,584; 5,571,670; 5,580,731; 5,571,670; 5,591,584; 5,624,802; 5,635,352; 5,594,118; 5,359,100; 5,124,246; and 5,681,697, each of which is hereby incorporated by reference.

In addition, other methods known in the art for detecting miRNA may be employed. For example, the methods described in U.S. Pat. Pub. Nos. US 2006/0292616 A1, US 2006/0228729, and US 2007/0050146 (the disclosures of which are all incorporated herein by reference in their entirety) may be used.

One method of detection is microarray analysis. In this method, multiple target sequences may be assayed within a single sample volume. Microarrays may be used to identify both precursor and mature miRNAs.

A preferred method of detection is quantitative RT-PCR. In this method, reverse transcription (RT) is used to convert the target RNA into cDNA, cDNA is then amplified and quantified using standard PCR methods. Primers used for reverse transcription or PCR amplification may include both conventional or modified primers, such as stem-loop RT primers, and LNA-containing primers. Quantitative PCR may be used in a single or multiplex format.

Additional methods for detection may use micro or nanotechnologies to increase measurement sensitivity, precision, dynamic range, and/or to increase throughput. These methods may include chemical, electrical, and/or optical detection methods. These may also be used in combination with the above mentioned conventional molecular detection methods (i.e. hybridization or RT-PCR).

To carry out the diagnostic methods described herein, the miRNA may be harvested from a biological fluid sample. Biological fluids include, but are not limited to, blood, serum and plasma. In some embodiments, the biological sample comprises exosomes or isolated microvesicles. The biological sample can be used (i) directly as obtained from the source or (ii) following a pre-treatment to modify the character of the sample. Thus, the sample can be pre-treated prior to use by, for example, preparing plasma from blood, isolating nucleic acid, concentrating liquids, inactivating interfering components, removing heparin from the sample, adding reagents, and the like. Samples also can be pretreated to digest, restrict or render double stranded nucleic acid sequences single stranded. Moreover, samples may be pretreated to accumulate, purify, amplify or otherwise concentrate sequences that may be contained therein.

Therapeutic Uses

Also described herein are methods of treating a human subject diagnosed with idiopathic pulmonary fibrosis (IPF) according to any of the diagnostic methods described herein, wherein the method comprises administering a therapeutic agent to the patient to treat IPF. Optionally, the method further includes one or more additional steps of detecting the level or expression pattern of one or more microRNA post-treatment, to monitor therapeutic efficacy of the treatment and (if warranted) adjust the dose, dosing schedule, or treatment agents.

In another aspect, a method of treating a human subject identified as having an abnormal level of one or more IPF-associated microRNAs in a blood sample of the subject, comprising administering a therapeutic agent to the subject to treat IPF is provided. The term “abnormal level” as used herein refers to a level of one or more microRNAs present in a blood sample of a subject suffering from IPF that is either increased or decreased when compared to a predetermined criterion as that term is defined herein; or falls within a predetermined range indicative of IPF as that term is defined herein. Alternatively, the abnormal level falls outside of a predetermined range indicative of healthy patients, or patients without IPF. The level of a differentially expressed microRNA may refer to either the uncorrected (raw) or normalized abundance of a microRNA in a sample. Comparisons of microRNA levels may consider the uncorrected quantified abundance of a given microRNA relative to an uncorrected reference value. Alternatively, the abundance of a given microRNA may be expressed as a ratio relative to one or more additional microRNAs (or other internal controls) in that sample. In such a case, this “normalized” ratio would be compared relative to a similar “normalized” reference value from a sample of healthy patients (or patients without IPF).

In some embodiments, the therapeutic agent is selected from the group consisting steroids (including but not limited to prednisolone), cytotoxic agents (including but not limited to azathioprine and cyclophosphamide), bardoxolone, LPA antagonists (including but not limited to AM152); Torisel (temsirolimus); PI3K inhibitors; pentraxin (including but not limited to Pentraxin-2 (PTX-2 or PRM-151)); MEK inhibitors (including but not limited to ARRY-162 and ARRY-300); p38 inhibitors; PAI-1 inhibitors (including but not limited to Tiplaxtinin); agents that reduce the activity of transforming growth factor-beta (TGF-β) (including but not limited to pan TGF-β neutralizing antibodies, such as GC-1008 (Genzyme/MedImmune); anti-TGF-β2 mAbs, such as lerdelimumab (CAT-152; Trabio, Cambridge Antibody); anti-TGF-β1 antibodies, such as metelimumab (CAT-192, Cambridge Antibody); small molecule TGF-βR1 inhibitors, such as LY-2157299 (Eli Lilly); ACU-HTR-028 (Opko Health)) including antibodies that target one or more TGF-β isoforms, inhibitors of TGF-β receptor kinases TGFBR1 (ALK5) and TGFBR2, and modulators of post-receptor signaling pathways; modulators of chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610; PS-433540; S-0139; sitaxentan sodium; TBC-3711; zibotentan); agents that reduce the activity of connective tissue growth factor (CTGF) (including but not limited to FG-3019, FibroGen), and also including other CTGF-neutralizing antibodies, such as FG-3019; matrix metalloproteinase (MMP) inhibitors (including but not limited to MMPI-12, PUP-1 and tigapotide triflutate, and doxycycline, marimastat, and cipemastat); agents that reduce the activity of epidermal growth factor receptor (EGFR) including but not limed to erlotinib, gefitinib, BMS-690514, cetuximab, antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Ra) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573 or rapamycin); agents that reduce levels of copper such as tetrathiomolybdate; agents that reduce oxidative stress including N-acetyl cysteine and tetrathiomolybdate; and interferon gamma. Also contemplated are agents that are inhibitors of phosphodiesterase 4 (PDE4) (including but not limited to Roflumilast); inhibitors of phosphodiesterase 5 (PDE5) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton). Further contemplated are compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil, P-1894B, and safironil) and peroxisome proliferator-activated receptor (PPAR)-gamma agonists (including but not limited to pioglitazone and rosiglitazone). The method discloseds can comprise administering an agent as disclosed directly above and/or an agent selected from BG-12, chemokine activity modulators (including but not limited to CNTO 888, an antibody targeting CCL2), Lysl oxidase inhibitors (including but not limited to AB0024/GS-6624, an antibody targeting human lysyl oxidase-like 2), NOX4 inhibitors (including but not limited to GKT137831, a selective NOX 1/4 inhibitor), angiotensin II receptor antagonists (including but not limited to lorsartan), inhibitors or Wnt-beta catenin signaling agents (including but not limited to ICG-001); JNK inhibitors (including but not limited to CC930); IL-4/IL-13 antibody/soluble receptors (including but not limited to SAR156597), and a deuterated pirfenidone (as described e.g., in WO 09/035,598 and having one to fourteen deuterium atoms replacing a hydrogen atom in pirfenidone).

For example for LPA1 receptor antagonists, the agent can be

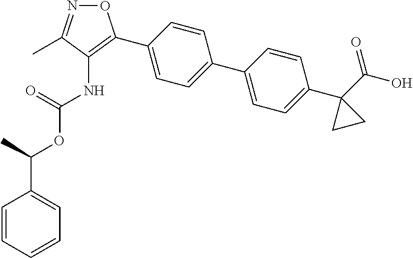 [SMILES: Cc1noc(-c2ccc(-c3ccc(C4(C(=O)O)CC4)cc3)cc2)c1NC(=O)O[C@H](C)c1ccccc1]

or one or more of (4′-{3-Methyl 4-[1-trifluoromethyl-phenyl)-ethoxycarbonoamino]-isoxazol-5-yl}-biphenyl-4-yl)-acetic acid (Compound 1); (4′-{3-Methyl-4-[1-(3-triflouomethyl-phenyl)-ethoxycarbonylamino]isoxazol-5-yl}-biphenyl-4-yl)-acetic acid (Compound 2); (4′-{4-[1-(2,4-Dichloro-phenyl)-ethoxycarbonylamino]-3-methyl-isoxazol-5-yl}-biphenyl-4-yl)-acetic acid (Compound 3) (4′-{4-[1-(2-flouoro-pehnyl)-ethylcabonylamino]-3-methyl-isoxazol-5-yl}-biphenyl-4-yl)-acetic acid (Compound 4); (4′-{4-[1-(3-Bromo-phenyl)-ethoxycarbonylamino]-3-methyl-isoxazol-5-yl}-biphenyl-4-yl)-acetic acid (Compound 5); (4′-{4-[1-(2-Methoxy-phenyl)-ethoxycarbonylamino]-3-methyl-isoxazol-5-yl}-biphenyl-4-yl)-acetic acid (Compound 6); (4′-{4-[1-(2-Chloro-phenyl)-ethoxycabonylamino]-3-methyl-Isoxazol-5-yl}-6-methoxy-biphenyl-3-yl)acetic acid (Compound 7); 4′-{4-[1-(2-Chloro-pheynyl-ethoxycarbonylamino]-3-methyl-isoxazol-5-yl}-biphenyl-4-carboxylic acid (Compound 8); 4′-{4-[1-(2-chloro-phenyl)-ethoxycarbonylamino]-3-methyl-isoazol-5-yl}-biphenyl-2-carboxylic acid (Compound 9); (4′-{4-[1-(2-Chloro-phenyl)-ethoxycarbonylamino]-3-methyl-isoxazol-5-yl}-biphenyl-2-yl)-acetic acid (Compound 10); (4′-{4-[1-(2-Chloro-phenyl)-ethoxycarbonylamino]-3-methyl-isoxazol-5-yl}-biphenyl-4-yl)-acetic acid (Compound 11); (4′-{4-[1-(2-Chloro-phenyl)-ethoxycarbonylamino]-3-methyl-isoxazol-5-yl}-biphenyl-3-yl)-acetic acid (Compound 12); 3-(4′-{4-[1-(2-Chloro-phenyl)-ethoxycarbonylamino]-3-methyl-isoxazol-5-yl}-biphenyl-4-yl)-propionic acid (Compound 13); (4′-{4-[1-(2-Chloro-phenyl)-ethoxycarbonylamino]-3-methyl-isoxazol-5-yl}-6-fluoro-biphenyl-3-yl)-acetic acid (Compound 14); (4′-{4-[1-(Chloro-phenyl)-ethoxycarbonylamino]-3-methyl-isoxazol-5-yl}-4-fluoro-biphenyl-3-yl)-acetic acid (Compound 15); (4′-{4-[1-(2-Chloro-phenyl)-ethoxycarbonylamino]-3-methyl-isoxazol-5-yl}-biphenyl-4-yl)-acetic acid methyl ester (Compound 16); 2-(4′-4-[1-(2-(Chloro-phenyl)-ethoxycarbonylamino]-3-methyl-isoxazol-5-yl}-biphenyl-4-yl)-propionic acid ethyl ester (Compound 17); 2-(4′-{4-[1-(2-Chloro-phenyl)-ethoxycarbonylamino]-3-methyl-isoxazol-5-yl}-biphenyl-4-yl)-propionic acid (Compound 20); 4-(4′-{4-[1-(2-Chloro-phenyl)-ethoxycarbonylamino]-3-methyl-isoxazole-5-yl}-biphenyl-4-yl)-butyric acid (Compound 21); 4′-{4-[1-(2-Chloro-phenyl)-ethoxycarbonylamino]-3-methyl-isoxazol-5-yl}-biphenyl-3-carboxylic acid (Compound 22); (4′-{4-[1-(4-Chloro-2-fluoro-phenyl)-ethoxycarbonylamino]-3-methyl-isoxazol-5-yl}-biphenyl-4-yl)-acetic acid (Compound 23); (4′-{4-[(R)-1-(2-Fluoro-phenyl)-ethoxycarbonylamino]-3-methyl-isoxazol-5-yl}-biphenyl-3-yl)-acetic acid (Compound 24); (4′-{4-[(R)-1-(2-Fluoro-phenyl)-ethoxycarbonylamino]-3-methyl-isoxazole-5-yl}-biphenyl-4-yl)-acetic acid (Compound 25); 2-(4′-{4-[1-(2-Fluoro-phenyl)-ethoxycarbonylamino]-3-methyl-isoxazol-5-yl}-biphenyl-4-yl)-2-methyl-propionic acid (Compound 26); (4′-{4-[(R)-1-(2-Chloro-phenyl)-ethoxycarbonylamino]-3-methyl-isoxazol-5-yl}-biphenyl-4-yl)-acetic acid (Compound 27); 2-(4′-{4-[(R)-1-(2-Chloro-phenyl)-ethoxycarbonylamino]-3-methyl-isoxazol-5-yl}-biphenyl-4-yl)-2-methyl-propionic acid (Compound 28); 2-(4′-{4-[(R)-1-(2-Fluoro-phenyl)-ethoxycarbonylamino]-3-methyl-isoxazol-5-yl}-biphenyl-4-yl)-2-methyl-propionic acid (Compound 29); 2-(4′-{4-[(R)-1-(2-Fluoro-phenyl)-ethoxycarbonylamino]-3-methyl-isoxazol-5-yl}-biphenyl-4-yl)-propionic acid (Compound 30); 2-(4′-{4-[(R)-1-(2-Chloro-phenyl)-ethoxycarbonylamino]-3-methyl-isoxazole-5-yl}-biphenyl-4-yl)-propionic acid (Compound 31); (4′-(4-[1-(2,6-Dichloro-phenyl)-ethoxycarbonylamino]-3-methyl-isoxazole-5-yl)-biphenyl-4-yl)-acetic acid (Compound 32); 2-(4′-{4-[(R)-1-(2-Fluoro-phenyl)-ethoxycarbonylamino]-3-methyl-isoxazole-5-yl}-2′-methyl-biphenyl-4-yl)-propionic acid (Compound 33); (4′-{4-[(S)-1-(2-Fluoro-phenyl-ethoxycabonylamino]-3-methyl-isoxazol-5-yl}-biphenyl-4-yl)acetic acid (Compound 34); (4′-{4-[(S)-1-(2-Chloro-phenyl)-ethoxycarbonylamino]-3-methyl-isoxazol-5-yl}-biphenyl-4-yl)-acetic acid (Compound 35); {4′-[4-(2-Chloro-benzyloxycarbonylamino)-3-methyl-isoxazole-5-yl]-biphenyl-4-yl}-acetic acid (Compound 36); {4′-[3-Methyl-4-((R)-1-phenyl-ethoxycarbonylamino)-isoxazol-5-yl]-biphenyl-4-yl}-acetic acid (Compound 37); (4′-(4-{4-[1-(2,3-Difluoro-phenyl)-ethoxycarbonylamino]-3-methyl-isoxazole-5-yl}-biphenyl-4-yl)-acetic acid (Compound 38); (4′-{4-[1-(2,4-Difluoro-phenyl)-ethoxycarbonylamino]-3-methyl-isoxazol-5-yl}-biphenyl-4-yl)-acetic acid (Compound 39); (4′-{4-[1-(2-Fluoro-4-methoxy-phenyl)-ethoxycarbonylamino]-3-methyl-isoxazole-5-yl]-biphenyl-3-yl}-acetic acid (Compound 40); (4′-{4-[1-(2,5-Difluoro-phenyl)-ethoxycarbonylamino]-3-methyl-isoxazole-5-yl}-biphenyl-4-yl)-acetic acid (Compound 41); (4′-{4-[1-(2,6-Difluoro-phenyl)-ethoxycarbonylamino]-3-methyl-isoxazole-5-yl}-biphenyl-4-yl)-acetic acid (Compound 42); {4′-[3-Methyl-4-((R)-1-phenyl-ethoxycarbonylamino)-isoxazole-5-yl]-biphenyl-3-yl}-acetic acid (Compound 43); 4′-[3-Methyl-4-((R)-1-phenyl-ethoxycarbonylamino)-isoxazol-5-yl]-biphenyl-4-carboxylic acid (Compound 44); {4′-[3-Methyl-4-((R)-1-phenyl-ethoxycarbonylamino)-isoxazol-5-yl]biphenyl-2-yl}-acetic acid (Compound 45) {4′-[3-Methyl-4-((R)-1-o-tolyl-ethoxycarbonylamino)-isoxazol-5-yl]-biphenyl-4-yl}-acetic acid (Compound 46); 2-(4′-{4-[(R,R)-1-(2-Chloro-phenyl)-ethoxycarbonylamino]-3-methyl-isoxazol-5-yl}-biphenyl-4-yl)-propionic acid (Compound 47); 2-(4′-{4-[(R,S)-1-(2-Chloro-phenyl)-ethoxycarbonylamino]-3-methyl-isoxazol-5-yl}-biphenyl-4-yl)-propionic acid (Compound 48); (3′-Chloro-4′-{4-[(R)-1-(2-Chloro-phenyl)-ethoxycarbonylamino]-3-methyl-isoxazole-5-yl}-biphenyl-4-yl)-acetic acid (Compound 49); 2-(4′-{4-[(R)-1-(2-Chloro-phenyl)-ethoxycarbonylamino]-3-methyl-isoxazol-5-yl}-biphenyl-4-yl)-butyric acid (Compound 50); (2′-Chloro-4′-{4-[(R)-1-(2-chloro-phenyl)-ethoxycarbonylamino]-3-methyl-isoxazol-5-yl}-biphenyl-4-yl)-acetic acid (Compound 51); (4-{4-[(R)-1-(2-Chloro-phenyl)-ethoxycarbonylamino]-3-methyl-isoxazol-5-yl}-2′-flouoro-biphenyl-4-yl)-acetic acid (compound 52); 4′-{4-[(R)-1-(2-Chloro-phenyl)-ethoxycarbonylamino]-3-methyl-isoxazol-5-yl}-biphenyl-4-carboxylic acid (compound 53); (4′-{4-[(R)-1-(3,5-Dibromo-phenyl)-ethoxycarbonylamino]-3-methyl-isozazol-5-yl}-biphenyl-4-yl)-acetic acid (compound 56); {4′-[3-Methyl-4-((S)-1-phenyl ethoxycarbonylamino)-isoxazol-5-yl]-biphenyl-4-yl}-acetic acid (Compound 57); 4′-{4-[(R)-1-(3-Hydroxy-phenyl)-ethoxycarbonylamino]-3-methyl-isoxazol-5-yl}-biphenyl-4-yl)-acetic acid (Compound 58); {4′-[3-methyl-4-(1-phenyl-ethoxycarbonylamino)-isoxazol-5-yl]-biphenyl-4-yl}-acetic acid (Compound 59); [5-4′-Cyanomethyl-biphenyl-4-yl)-3-methyl-isoxazol-4-yl]-carbamic acid (R)-1-(2-chloro-phenyl)-ethyl ester (Compound 61); [5-(4′-Cyanomethyl-biphenyl-4-yl-3-methyl-isoxazol-4-yl]-carbamic acid (R)-1-(2-fluoro-phenyl)-ethyl ester (Compound 62); {3-Methyl-5-[4′-(2H-tetrazol-5-ylmethyl)-biphenyl-4-yl]-isoxazol-4-yl}-carbamic acid (R)-1-(2-fluoro-phenyl)-ethyl ester (Compound 63); {3-Methyl-5-[4′-(2H-tetrazole-5-ylmethyl)-biphenyl-4-yl]-isoxazole-4-yl}-carbamic acid (R)-1-(2-chloro-phenyl)-ethyl ester (Compound 64); [5-(4′-Carbamimidoylmethyl-biphenyl-4-yl)-3-methyl-isoxazole-4-yl]-cabamic acid (R)-1-(2-fluoro-phenyl)-ethyl ester (Compound 65); {5-[4′-(2-Acetylamino-2-imino-ethyl-biphenyl-4-yl]-3-methyl-isoxazol-4-yl}-carbamic acid (R)-1-(2-fluoro-phenyl)-ethyl ester (Compound 66); and 2-(2-{4′-[3-Methyl-4-((R)-1-phenyl-ethoxycarbonylamino)-isoxazol-5-yl]-biphenyl-4-yl}-acetylamino)-ethanesulfonic acid.

In particular, the LPA1 receptor antagonist can have a structure of any one of formulae (I), (Ia), (II), (IIIa), (III), (IIIa), (IV), and (V) as disclosed in WO 2011/041462; a structure of any one of formulae (I), (II), and (III) as disclosed in WO 2010/68775; a structure of formula (I) as disclosed in US 2010/311799; a structure of formula (I) as disclosed in WO 2010/141761; a structure of any one of formulae (I), (II), (III), (IV) and (IV) as disclosed in WO 2010/141768; a structure of formula (I) as disclosed in US 2010/152257; a structure of any one of formulae (I), (II) and (III) as disclosed in WO 10/77882; a structure of formula (I) as disclosed in WO 10/77883; a structure of formula (I) as disclosed in US 2011/0082164; a structure of any one of formulae (I) and (II), as disclosed in WO 11/041,461; a structure of any one of compounds 1-79 or formula (I) as disclosed in US 2011/0082181; a structure of any one of formulae (I), (II), (III), (IV), (V), (VI), and (VI) as disclosed in WO 2011/041694; a structure of formula (I) as disclosed in WO 11/041,729; structure of any one of formulae (I), (II), (III), (IV), and (V) as disclosed in WO 11/017,350, each of which is incorporated by reference in its entirety. These and related LPA1 receptor antagonists, and methods of synthesizing them, are disclosed generally in the following patent publications: WO 2010/68775; US 2010/311799; WO 2010/141761; WO 2010/141768; US 2010/152257; WO 2010/77883; WO 2010/77882, US 2011/82164; WO 2011/41461; WO 2011/41462; US 2011/82181; WO 2011/41694; WO 2011/41729; WO 2011/17350, each of which is incorporated by reference in its entirety.

Additional LPA1 receptor antagonists contemplated include compounds of formulae (1), (2) and (5), and in particular compounds 101-169, as disclosed in U.S. Pat. No. 6,964,975 and US Patent Publication No. 2003/114505, each of which is incorporated by reference in its entirety. A specific compound from this family is

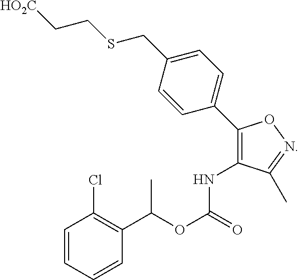 [SMILES: Cc1noc(-c2ccc(CSCCC(=O)O)cc2)c1NC(=O)OC(C)c1ccccc1Cl]

Still other LPA receptor antagonists contemplated include compounds disclosed in U.S. Pat. No. 7,517,996, and in particular a compound having a structure of formula (I), which is incorporated by reference in its entirety.

Still other LPA receptor antagonists contemplated include compounds disclosed in U.S. Pat. No. 7,288,558, and in particular compounds having a structure of formula (I), which is incorporated by reference in its entirety.

Also contemplated are agents that are PG D2 modulators, such as compounds having a structure of any one of formulae (I), (II), (III), (IV), (V), (VI), (VII), (VIII), and (IX), as disclosed in US 2011/0098302 or structure of formula (I), as disclosed in US 2011/0098352, each of which is incorporated by reference in its entirety.

Also contemplated are the following agents or classes of agents: one or more of nitric oxide (e.g., inhaled nitric oxide), a vitamin E and pentoxifylline combination (e.g., PTL-202 from Pacific Therapeutics), PXS25, desatinib (a multiple kinase inhibitor), PI3K/mTor dual inhibitor (e.g., BAY806946, XL765, GDC0980, GSK2126458, BEZ235, BGT226, PF04691502, PK1587, and/or SF1126), PI3K inhibitor (e.g., XL147, GDC0941, BKM120, PX866, ZSTK474, BYL719 (PI3K alpha), AMG319 (PI3K delta), CAL101 (PI3K delta), and/or GDC0032), 5-HT2A/B receptor antagonists (e.g., terguride), telomerase activator (e.g., TAT153), modulators (e.g., reducers) of chemokine activity (e.g., CNTO 888, an antibody that targets CCL2), Lysl oxidase inhibitors (e.g., AB0024/GS-6624, a humanized mAb targeting human lysyl oxidase-like 2), NOX4 inhibitor (e.g., GKT137831, a selective Nox 1/4 inhibitor), angiotensin II receptor antagonist (e.g., lorsartan), an anti avP6 integrin agent, and pentraxin (e.g., serum amyloid P, PTX-2, or PRM-151).

Also contemplated are agents that are pirfenidone analogs, such as compounds having a structure of any one of formulae (I), (II), (III), (IV), and (V), as disclosed in WO 10/085,805, the disclosure of which is incorporated by reference in its entirety. The synthesis of the pirfenidone analog compounds disclosed in WO 10/085,805 are further described in U.S. Patent Publication No. 2007/0049624 (US national stage of WO 05/0047256), International Publication No. WO 03/068230, WO 08/003,141, WO 08/157,786, or in U.S. Pat. Nos. 5,962,478; 6,300,349; 6,090,822; 6,114,353; Re. 40,155; 6,956,044; or 5,310,562, each of which is incorporated by reference in its entirety.

The pirfenidone analogs disclosed in WO 10/085,805, have structures of formulae (I), (II), (III), (IV), or (V):

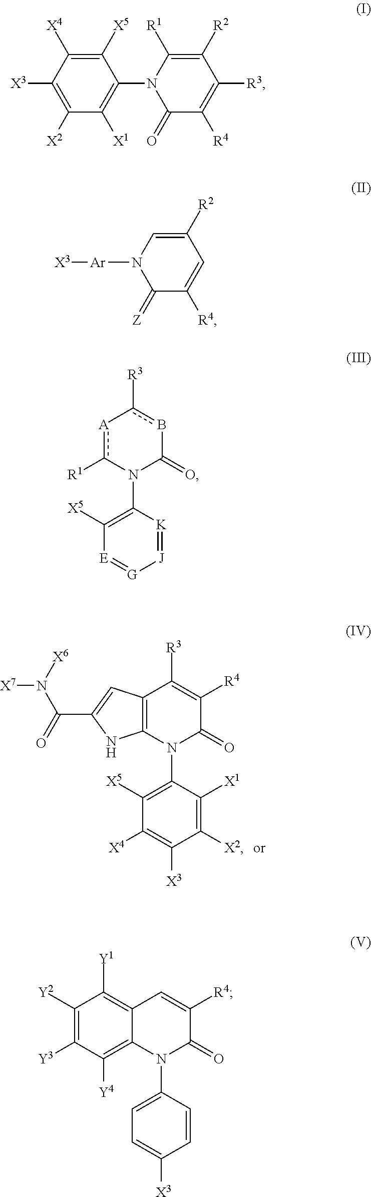

wherein A is N or CR²; B is N or CR⁴; E is Nor CX⁴; G is N or CX³; J is N or CX²; K is N or CX¹; a dashed line is a single or double bond, R¹, R², R³, R⁴, X¹, X², X³, X⁴, X⁵, Y¹, Y², Y³, and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted C₁-C₁₀ alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, substituted C₁-C₁₀ alkoxyalkyl, C₁-C₁₀ carboxy, substituted C₁-C₁₀ carboxy, C₁-C₁₀ alkoxycarbonyl, substituted C₁-C₁₀ alkoxycarbonyl, CO-uronide, CO-monosaccharide, CO-oligosaccharide, and CO-polysaccharide; X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; Ar is pyridinyl or phenyl; and Z is O or S. In some embodiments, A is N or CR²; B is N or CR⁴; E is N, N⁺X⁴ or CX⁴; G is N,N⁺X³ or CX³; J is N,N⁺X² or CX²; K is N,N⁺X¹ or CX¹; a dashed line is a single or double bond, R¹, R², R³, R⁴, X¹, X², X³, X⁴, X⁵, Y¹, Y², Y³, and Y⁴ are independently selected from the group consisting of H, deuterium, optionally substituted C₁-C₁₀ alkyl, optionally substituted C₁-C₁₀ deuterated alkyl, optionally substituted C₁-C₁₀ alkenyl, optionally substituted C₁-C₁₀ thioalkyl, optionally substituted C₁-C₁₀ alkoxy, optionally substituted cycloalkyl, optionally substituted heterocycloalkyl, optionally substituted heteroalkyl, optionally substituted aryl, optionally substituted heteroaryl, optionally substituted amido, optionally substituted sulfonyl, optionally substituted amino, optionally substituted sulfonamido, optionally substituted sulfoxyl, cyano, nitro, halogen, hydroxyl, SO₂H₂, optionally substituted C₁₋C₁₀ alkoxyalkyl, optionally substituted C₁₋C₁₀-carboxy, optionally substituted C₁₋C₁₀ alkoxycarbonyl, CO-uronide, CO-monosaccharide, CO-oligosaccharide, and CO-polysaccharide; X⁶ and X⁷ are independently selected from the group consisting of hydrogen, optionally substituted aryl, optionally substituted heteroaryl, optionally substituted cycloalkyl, optionally substituted heterocycloalkyl, optionally substituted alkylenylaryl, optionally substituted alkylenylheteroaryl, optionally substituted alkylenylheterocycloalkyl, optionally substituted alkylenylcycloalkyl, or X⁶ and X⁷ together form an optionally substituted 5 or 6 membered heterocyclic ring; and Ar is optionally substituted pyridinyl or optionally substituted phenyl; and Z is O or S.

The pirfenidone administered in the methods disclosed herein can be deuterated. The pirfenidone can be a mixture of deuterated forms of pirfenidone, a single deuterated form, or a mixture of deuterated form (or forms) and non-deuterated pirfenidone. Contemplated deuterated pirfenidone includes pirfenidone with 1, 2, 3, 4, 5, 6, 7, 8, 9, 10, 11, 12, 13, or 14 deuterium atoms. The phenyl ring of pirfenidone can be deuterated with 1, 2, 3, 4, or 5 deuterium atoms. Additionally or alternatively, the methyl of pirfenidone can be deuterated with 1, 2, or 3 deuterium atoms. Additionally or alternatively, the pyridone ring hydrogens can be substituted with 1, 2, 3, or 4 deuterium atoms. Multiple different deuterated pirfenidone forms and methods of synthesizing the various deuterated pirfenidone forms are disclosed in WO 09/035,598, the disclosure of which is incorporated by reference in its entirety.

Combinations of one or more of the foregoing agents are also contemplated.

In one embodiment the invention provides methods of treating a subject having IPF, for example, by administering to the subject an effective amount of an agent which modulates the level of at least one miRNA in a target cell. In some embodiments, the agent increases or stimulates the expression or activity of a miRNA in a mammalian subject (i.e., a miRNA enhancer). In other embodiments, the agent decreases or inhibits the expression or activity of a miRNA in a mammalian subject (i.e., an miRNA or miRNA inhibitor).

As used herein, “an agent which modulates the level of miRNA” indicates that the agent, when administered to a sample or subject increases or a decreases in the measured value of at least one miRNA. In some embodiments the miRNA is increased or decreased by an amount between 1-fold and 20-fold, or more than 20-fold. In some particular embodiments the miRNA is increased or decreased by 1-fold, 2-fold, 3-fold, 4-fold, 5-fold, 7-fold, 9-fold, 10-fold, 12-fold, or 15-fold, or more. In other embodiments the miRNA is increased or decreased by 5%, 10%, 20%, 30%, 40%, 50%, 60%, 70%, 80%, 90%, 100%, 150%, 200%, 300%, or more.

miRNA enhancers are molecules, e.g., nucleic acid molecules, which act to increase the level of a miRNA gene product in a cell. In one variation, a miRNA enhancer comprises a sequence of a miRNA, or a variant thereof. In another variation, the miRNA molecule is a synthetic molecule. In another variation, the miRNA molecule comprises one or more stabilizing mutations. The miRNA sequence may be 12-100 nucleotides in length. For example, the miRNA sequence may comprise 20-80, 20-70, 20-60, 20-50, 20-40, 21-23, 21-25 12-33, 18-24, 18-26, or 21-23 nucleotides. In some embodiments, the miRNA sequence may comprise 15, 16, 17, 18, 19, 20, 21, 22, 23, 24, 25, 26, 27, 28, 29, or 30 nucleotides. The sequence of the miRNA may be the first 13-33, or 21-25 nucleotides of the pre-miRNA. In some embodiments, the sequence of the miRNA may be the last 13-33 or 21-25 nucleotides of the pre-miRNA.

In another variation, the miRNA enhancer comprises a sequence of a pri-miRNA or a variant thereof. The pri-miRNA sequence may comprise from 30-300, 35-375, 45-250, 55-200, 70-150 or 80-100 nucleotides. The pri-miRNA may also comprise a miRNA and the complement thereof, and variants thereof. The pri-miRNA may form a hairpin structure. The hairpin may comprise a first and second nucleic acid sequence that are substantially complimentary. The first and second nucleic acid sequence may be from 37-50 nucleotides. The first and second nucleic acid sequence may be separated by a third sequence of from 8-12 nucleotides. The hairpin structure may have a free energy less than −25 Kcal/mole as calculated by the Vienna algorithm with default parameters, as described in Hofacker et al., Monatshefte f. Chemie 125:167-188 (1994), the contents of which are incorporated herein. The hairpin may comprise a terminal loop of 4, 5, 6, 7, 8, 9, 10, 11. 12, 13, 14, 15, 16, 17, 18, 19, 20, 21, 22, 23, 24, or 25 nucleotides.

In contrast, miRNA inhibitors decrease or inhibit the expression or activity of a miRNA in a mammalian subject. In some embodiments, the miRNA inhibitor is antagomir. As used herein, the term “antagomir” is an anti-miRNA molecule that is capable of blocking the activity of a miRNA. The antagomir may comprise a total of 12-50 or 8-50, or 8-40, or 5-40 nucleotides in length. In some embodiments, the antagomir comprises a total of at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55 or 60 nucleotides. The sequence of an antagomir may comprise the complement of a sequence of a miRNA such that, e.g., the anti-miRNA binds to the miRNA to block its activity.

In some embodiments, the antagomirs comprise one or more non-naturally occurring or modified nucleotides. The one or more modified nucleotide analog may be located for example at the 5′-end and/or the 3′-end of the nucleic acid molecule or within the nucleic acid molecule. Representative examples of nucleotide analogs may be selected from sugar- or backbone-modified ribonucleotides. It should be noted, however, that nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. In a preferred embodiment, the antagomir comprises a 2′-O methyl modification. In a highly preferred embodiment, the antagomir comprises a 2′,5′ locked nucleic acid (LNA) modification.

Modifications of the ribose-phosphate backbone may be done for a variety of reasons, e.g., to increase the stability and half-life of such molecules in physiological environments or as probes on a biochip. Mixtures of naturally occurring nucleic acids and analogs may be made; alternatively, mixtures of different nucleic acid analogs, and mixtures of naturally occurring nucleic acids and analogs may be made. It will further be understood that combinations of modifications (e.g., modifications to backbone linkages and 2′O modifications) may be made to the same nucleic acid molecule. Stabilizing alterations may include the use of nonionic DNA analogs, such as alkyl- and aryl-phosphonates (in which the charged phosphonate oxygen is replaced by an alkyl or aryl group), phosphodiester and alkylphosphotriesters, in which the charged oxygen moiety is alkylated.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al., 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et al. 2005 Cell 120-15). Similarly, nucleotides 1-7 or 2-8 were used to identify and validate targets by Krek et al. (2005, Nat. Genet. 37-495).

Nucleic acid inhibitors of an miRNA have complementarity to the miRNA molecule whose level is to be inhibited. In one embodiment, the inhibitor and the miRNA are 100% complementary over their full length (i.e., are complementary at 100% of the nucleotides of the miRNA molecule). In another embodiment, the inhibitor and the miRNA molecule are 95%, 90%, 85% or 80% complementary over their full length. In embodiments where the molecules are less than 100% complementary, preferably, the 2, 3, 4, 5, 6, 7, 8, 9, or 10 bases at the 5′ end of the miRNA molecule are complementary to the nucleotides present in the inhibitor at the corresponding position; mismatching may occur at other positions and the desired level of complementarity achieved.

Kits

In another embodiment, kits are provided which contain the necessary reagents to carry out the assays of the present invention. In one embodiment, the invention provides a compartment kit to receive, in close confinement, one or more containers which comprises a means of detecting a change in the level of one, two, three, four, five, six, seven or more microRNA correlated with a diagnosis of IPF in a blood sample from the subject. In some embodiments, the kit comprises a sample collection component with specific tubes and buffers, a miRNA extraction component, miRNA quantitative RT-PCR components with deliberate enzymes and primers and one or more containers comprising primers capable of specifically and quantitatively amplifying any of the IPF-associated miRNAs described herein. The term “specifically amplify” as used herein means that the primers in the kit amplify the IPF-associated miRNA but do not substantially amplify other miRNAs of non-homologous sequence. Quantitative RT-PCR kits may be in single, multiple (multiplex), or in a panel of parallel assays.

In another embodiment, kits are provided which contain the necessary reagents to carry out the assays of the present invention. In one embodiment, the invention provides a compartment kit to receive, in close confinement, one or more containers which comprises a means of detecting a change in the level of one, two, three, four, five, six, seven or more microRNA correlated with a diagnosis of IPF in a blood sample from the subject. In some embodiments, the kit comprises a sample collection component with specific tubes and buffers, a miRNA extraction component, miRNA reverse transcription and/or labeling components (as appropriate), and a component with appropriate primers or customized specific miRNA hybridization components. One or more containers comprising probes or arrays of probes capable of specifically hybridizing to any of the IPF-associated miRNAs described herein. In other embodiments, the kit comprises one or more microarrays comprising probes capable of specifically detecting any of the IPF-associated miRNAs described herein, and includes the additional components for detection that may include chemical, electrical, and/or optical detection methods. The term “specifically hybridize” as used herein means that the probes in the kit hybridize under stringent conditions to the IPF-associated miRNA but not substantially to other miRNAs of non-homologous sequence. In other embodiments, the kit comprises one or more microarrays comprising probes capable of specifically detecting any of the IPF-associated miRNAs described herein.

In some embodiments, the kit comprises a means for sample collection (e.g., collection tubes and buffers for maintaining microRNA integrity in the sample); instructions and materials for the extraction of microRNA from the sample; instructions and appropriate buffers, substrates and enzymes for microRNA reverse transcription; and instructions and materials (e.g., DNA polymerase, nucleotide substrates, PCR buffer, detection components and PCR primers universal or microRNA specific PCR primers) for microRNA amplification and quantification.

In detail, a compartment kit includes any kit in which reagents are contained in separate containers. Such containers include small glass containers, plastic containers or strips of plastic or paper. Such containers allow one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibody or antibodies used in the assay, containers which contain wash reagents (such as phosphate-buffered saline, Tris buffers, and the like), and containers which contain the reagents used to detect the bound antibody or probe. Types of detection reagents include nucleic acid probes or primers, either of which may be labeled.

Diagnostic Systems

A “diagnostic system” is any system capable of carrying out the methods of the invention, including computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. Specifically contemplated herein are a system adapted to perform the steps of any of the methods described herein, and a computer program product comprising computer-executable instructions embodied in a computer-readable medium for performing the steps of any of the methods described herein.

Tests to measure and compare levels of one, two, three, four, five, six, seven or more microRNA can be implemented on a wide variety of diagnostic test systems. Diagnostic test systems are apparatuses that typically include means for obtaining test results from biological samples. Examples of such means include modules that automate the testing (e.g., biochemical, immunological, nucleic acid detection assays). Some diagnostic test systems are designed to handle multiple biological samples and can be programmed to run the same or different tests on each sample. Diagnostic test systems typically include means for collecting, storing and/or tracking test results for each sample, usually in a data structure or database. Examples include well-known physical and electronic data storage devices (e.g., hard drives, flash memory, magnetic tape, paper print-outs). It is also typical for diagnostic test systems to include means for reporting test results. Examples of reporting means include visible display, a link to a data structure or database, or a printer. The reporting means can be nothing more than a data link to send test results to an external device, such as a data structure, data base, visual display, or printer.

Still another embodiment of the invention is a computer readable medium having computer executable instructions for diagnosing IPF, the computer readable medium comprising: a routine, stored on the computer readable medium and adapted to be executed by a processor, to store one or more predetermined criteria or ranges; and a routine stored on the computer readable medium and adapted to be executed by a processor to compare the level of one, two, three, four, five, six, seven or more microRNA in a test sample data to its respective predetermined criterion or predetermined range to diagnose IPF.

A computer-readable storage medium can comprise a data storage material encoded with computer readable data or data arrays which, when using a machine programmed with instructions for using said data, is capable of use for a variety of purposes, such as, without limitation, subject information relating to diagnosing IPF. Measurements of microRNA in a sample can be implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design. The output may include (a) the level of one, two, three, four, five, six, seven or more microRNAs and (b) the respective one or more predetermined criteria or predetermined ranges. Preferably the levels of at least 1, 2, 3, 4, 5, 6, 7, 8, 9, 10, 11, 12, 13, 14, 15, 16, 17, 18, 19 or 20 different microRNAs are detected, such that the output includes at least 2, 3, 4, 5, 6, 7, 8, 9, 10, 11, 12, 13, 14, 15, 16, 17, 18, 19 or 20 different levels and predetermined criteria or ranges. Analogously, levels of at least 21, 22, 23, 24, 25, 26, 27, 28, 29, 30, 35, 40 or more different microRNAs may be detected.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The data comparison system of the invention may also be considered to be implemented as a computer-readable storage medium, configured with a computer program, where the storage medium so configured causes a computer to operate in a specific and predefined manner to perform various functions described herein. Levels of microRNA in a sample can then be determined and compared to a predetermined criterion or range as described herein.

The invention is further described in the following additional embodiments:

1A. A method of diagnosing idiopathic pulmonary fibrosis (IPF) in a human subject comprising detecting in a blood sample of from the subject the level of one or more microRNAs, wherein an increase or decrease in the level of the one or more microRNA relative to a predetermined criterion is indicative of a diagnosis of IPF.

2A. The method of embodiment 1A, further comprising the step of comparing the level of the microRNA to a predetermined criterion or range.

3A. The method of embodiment 1A or embodiment 2A, wherein the one or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2 (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), and combinations thereof.

4A. The method of embodiment 1A or embodiment 2A, wherein the one or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), and combinations thereof.

5A. The method of claim 1A or claim 2A, wherein the one or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), and combinations thereof.

6A. The method of embodiment 1A or embodiment 2A, wherein the one or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), and combinations thereof.

7A. The method of any one of embodiments 1A-6A, wherein the level or expression pattern of at least 2 different microRNAs is detected.

8A. The method of any one of embodiments 1A-6A, wherein the level or expression pattern of at least 10 different microRNA is detected.

9A. The method of any one of embodiments 1A-6A, wherein the level or expression pattern of at least 20 different microRNA is detected.

10A. The method of any one of embodiments 1A-4A, wherein the presence or an increased level of one or more microRNA is detected, relative to a predetermined criterion, is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), and combinations thereof.

11A. The method of any one of embodiments 1A-4A, wherein the presence or an increased level of one or more microRNA is detected, relative to a predetermined criterion, is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11); miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21) and combinations thereof.

12A. The method of any one of embodiments 1A-4A, wherein the presence or an increased level of one or more microRNA is detected, relative to a predetermined criterion, is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11); miR-193b (SEQ ID NO:12), miR-34a# (SEQ ID NO:13), miR-548a-3p (SEQ ID NO:14) and combinations thereof.

13A. The method of any one of c embodiments 1A-4A, wherein the presence or an increased level of one or more microRNA is detected, relative to a predetermined criterion, is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11) and combinations thereof.

14A. The method of any one of embodiments 1A-13A, wherein the absence or a decreased level of one or more microRNA is detected, relative to a predetermined criterion, indicative of a diagnosis of IPF, and the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d# (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), and combinations thereof.

15A. The method of any one of embodiments 1A-11A, wherein the absence or a decreased level of one or more microRNA is detected, relative to a predetermined criterion, indicative of a diagnosis of IPF, and the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88),) and combinations thereof.

16A. The method of any one of embodiments 1A-11A, wherein the absence or a decreased level of one or more microRNA is detected, relative to a predetermined criterion, indicative of a diagnosis of IPF, and the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61) and combinations thereof.

17A. The method of any one of embodiments 1A-11A, wherein the absence or a decreased level of one or more microRNA is detected, relative to a predetermined criterion, indicative of a diagnosis of IPF, and the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), and combinations thereof.

18A. The method of any of embodiments 1A-17A, wherein the sample is selected from the group consisting of whole blood, serum, plasma, exosomes and isolated microvesicles.

19A. The method of any one of embodiments 1A-17A, wherein the method further comprises administering a therapeutic agent to the subject.

20A. A method of treating a human subject diagnosed with idiopathic pulmonary fibrosis (IPF) according to any of embodiments 1A-18A comprising administering a therapeutic agent to the subject to treat IPF.

21A. A method of treating a human subject identified as having idiopathic pulmonary fibrosis (IPF) or at risk of IPF based on an abnormal level of one or more IPF-associated microRNAs in a blood sample of the subject, comprising administering a therapeutic agent to the subject to treat IPF.

22A. The method of embodiment 20A or embodiment 21A, wherein the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2 (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), or combinations thereof.

23A. The method of embodiment 20A or embodiment 21A, wherein the level or expression pattern of at least 2 different microRNAs is detected.

24A. The method of embodiment 20A or embodiment 21A, wherein the level or expression pattern of at least 10 different microRNA is detected.

25A. The method of embodiment 20A or embodiment 21A, wherein the level or expression pattern of at least 20 different microRNA is detected.

26A. The method of embodiment 21A, wherein the sample is selected from the group consisting of whole blood, serum, plasma, exosomes and isolated micro vesicles.

27A. The method of any of embodiments 19A-26A, wherein the therapeutic agent is an oligonucleotide that decreases the activity or level of expression of one or more of the microRNA in the subject.

28A. The method of any of embodiments 19A-26A, wherein the therapeutic agent is an oligonucleotide that increases the activity or level of expression of one or more of the microRNA in the subject.

29A. The method of any of embodiments 19A-26A, wherein the therapeutic agent is an anti-fibrotic agent.

30A. The method of embodiment 29A, wherein the anti-fibrotic agent is pirfenidone.

31A. The method of any of embodiments 19A-26A, wherein the therapeutic agent is selected from the group consisting of steroids (including but not limited to prednisolone), cytotoxic agents (including but not limited to azathioprine and cyclophosphamide), bardoxolone, LPA agonists (including but not limited to AM152); Torisel (temsirolimus); PI3K inhibitors; pentraxin or serum amyloid P (including but not limited to Pentraxin-2 (PTX-2 or PRM-151)); MEK inhibitors (including but not limited to ARRY-162 and ARRY-300); p38 inhibitors; PAI-1 inhibitors (including but not limited to Tiplaxtinin); agents that reduce the activity of transforming growth factor-beta (TGF-β) (including but not limited to GC-1008 (Genzyme/MedImmune); lerdelimumab (CAT-152; Trabio, Cambridge Antibody); metelimumab (CAT-192, Cambridge Antibody,); LY-2157299 (Eli Lilly); ACU-HTR-028 (Opko Health)) including antibodies that target one or more TGF-β isoforms, inhibitors of TGF-β receptor kinases TGFBR1 (ALK5) and TGFBR2, and modulators of post-receptor signaling pathways; chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610; PS-433540; S-0139; sitaxentan sodium; TBC-3711; zibotentan); agents that reduce the activity of connective tissue growth factor (CTGF) (including but not limited to FG-3019, FibroGen), and including other CTGF-neutralizing antibodies; matrix metalloproteinase (MMP) inhibitors (including but not limited to MMPI-12, PUP-1 and tigapotide triflutate); agents that reduce the activity of epidermal growth factor receptor (EGFR) including but not limed to erlotinib, gefitinib, BMS-690514, cetuximab, antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Ra) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573 or rapamycin); agents that reduce levels of copper such as tetrathiomolybdate; agents that reduce oxidative stress including N-acetyl cysteine and tetrathiomolybdate; and interferon gamma, inhibitors of phosphodiesterase 4 (PDE4) (including but not limited to Roflumilast); inhibitors of phosphodiesterase 5 (PDE5) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton), compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil, P-1894B, and safironil) and peroxisome proliferator-activated receptor (PPAR)-gamma agonists (including but not limited to pioglitazone and rosiglitazone), and combinations thereof.

32A. A kit to be used in the diagnosis of subjects having idiopathic pulmonary fibrosis (IPF) comprising one or more probes that specifically hybridize to, or primers that specifically amplify, one or more microRNAs selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:11l), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), and combinations thereof.

33A. A diagnostic test system adapted for performing any of the methods of embodiments 1A-18A.

34A. The diagnostic test system of embodiment 32A or embodiment 33A comprising means for obtaining test results comprising the activity or level of one or more microRNA correlated with a diagnosis of idiopathic pulmonary fibrosis (IPF) in a blood sample of the subject; means for collecting and tracking test results for one or more individual blood sample; means for comparing the activity or level of one or more microRNA to a predetermined criterion; and means for reporting whether the activity or level of the one or more microRNA meets or exceeds the predetermined criterion

35A. A computer program product comprising computer-executable instructions embodied in a computer-readable medium for performing the steps of any of the methods of embodiments 1A-18A.

1B. A method of diagnosing idiopathic pulmonary fibrosis (IPF) in a human subject comprising detecting in a blood sample of from the subject the level of one, two, three, four, five six, seven or more microRNAs, wherein an increase or decrease in the level of the one or more microRNA relative to a predetermined criterion is indicative of a diagnosis of IPF.

2B. The method of embodiment 1B further comprising the step of comparing the level of the microRNA to a predetermined criterion or range.

3B. The method of embodiment 1B or embodiment 2B, wherein the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:11l), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a# (SEQ ID NO: 155), miR-106a (SEQ ID NO:156), miR-1227 (SEQ ID NO:157), miR-128 (SEQ ID NO: 158), miR-132 (SEQ ID NO:159), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO: 179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO: 191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof.

4B. The method of embodiment 1B or embodiment 2B, wherein the one, two, three, four, five, six, seven or more microRNA are selected from the group consisting of miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-197 (SEQ ID NO:10), miR-663B (SEQ ID NO:11), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), let-7d (SEQ ID NO:45), miR-361-5p (SEQ ID NO:48), miR-520e (SEQ ID NO:49), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-17# (SEQ ID NO:58), miR-32 (SEQ ID NO:60), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-20a (SEQ ID NO:89), miR-28-5p (SEQ ID NO:90), miR-148a (SEQ ID NO:91), let-7e (SEQ ID NO:93), miR-656 (SEQ ID NO:95), miR-362-3p (SEQ ID NO:96), miR-423-5p (SEQ ID NO:99), miR-652 (SEQ ID NO:100), miR-127-3p (SEQ ID NO:101), miR-495 (SEQ ID NO:102), miR-590-5p (SEQ ID NO:104), miR-103 (SEQ ID NO:105), miR-324-3p (SEQ ID NO:107), miR-598 (SEQ ID NO:110), miR-130a (SEQ ID NO:112), miR-502-3p (SEQ ID NO:113), miR-194 (SEQ ID NO:115), miR-221 (SEQ ID NO:116), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-411 (SEQ ID NO:124), miR-29b (SEQ ID NO:125), miR-340 (SEQ ID NO:127), miR-148b# (SEQ ID NO:128), miR-212 (SEQ ID NO:129), miR-152 (SEQ ID NO:130), miR-7 (SEQ ID NO:132), miR-543 (SEQ ID NO:133), miR-30d (SEQ ID NO:134), miR-213 (SEQ ID NO:135), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), let-7a# (SEQ ID NO: 155), miR-106a (SEQ ID NO:156), miR-1227 (SEQ ID NO:157), miR-128 (SEQ ID NO: 158), miR-132 (SEQ ID NO:159), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO: 179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO: 191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof.

5B. The method of embodiment 1B or embodiment 2B, wherein the one, two, three, four, five, six, seven or more microRNA are selected from the group consisting of miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-197 (SEQ ID NO:10), miR-663B (SEQ ID NO:11), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-572 (SEQ ID NO:35), miR-886-3p (SEQ ID NO:36), miR-26b (SEQ ID NO:40), miR-142-5p (SEQ ID NO:43), let-7d (SEQ ID NO:45), miR-361-5p (SEQ ID NO:48), miR-520e (SEQ ID NO:49), miR-148b (SEQ ID NO:53), miR-15b# (SEQ ID NO:55), miR-15b (SEQ ID NO:56), miR-17# (SEQ ID NO:58), miR-190 (SEQ ID NO:63), miR-15a# (SEQ ID NO:64), miR-181a (SEQ ID NO:66), miR-301a (SEQ ID NO:67), miR-374a (SEQ ID NO:68), miR-144# (SEQ ID NO:69), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-28-5p (SEQ ID NO:90), miR-148a (SEQ ID NO:91), let-7e (SEQ ID NO:93), miR-656 (SEQ ID NO:95), miR-362-3p (SEQ ID NO:96), miR-652 (SEQ ID NO:100), miR-127-3p (SEQ ID NO:101), miR-495 (SEQ ID NO:102), miR-590-5p (SEQ ID NO:104), miR-598 (SEQ ID NO:110), miR-130a (SEQ ID NO:112), miR-502-3p (SEQ ID NO:113), miR-194 (SEQ ID NO:115), miR-221 (SEQ ID NO:116), miR-335 (SEQ ID NO:119), miR-24-2# (SEQ ID NO:120), miR-130b (SEQ ID NO:121), miR-99b (SEQ ID NO:122), miR-411 (SEQ ID NO:124), miR-340# (SEQ ID NO:127), miR-148b# (SEQ ID NO:128), miR-212 (SEQ ID NO:129), miR-152 (SEQ ID NO:130), miR-7 (SEQ ID NO:132), miR-543 (SEQ ID NO:133), miR-30d (SEQ ID NO:134), miR-213 (SEQ ID NO:135), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), let-7a# (SEQ ID NO:155), miR-106a (SEQ ID NO: 156), miR-1227 (SEQ ID NO:157), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO: 179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO: 191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof.

6B. The method of embodiment 1B or embodiment 2B, wherein the one, two, three, four, five, six, seven or more microRNA are selected from the group consisting of let-7a (SEQ ID NO:173), let-7a# (SEQ ID NO:155), let-7d (SEQ ID NO:45), miR-106a (SEQ ID NO:156), let-7e (SEQ ID NO:93), miR-122 (SEQ ID NO:22), let-7f (SEQ ID NO:174), miR-1227 (SEQ ID NO:157), let-7g (SEQ ID NO:72), miR-128 (SEQ ID NO:158), miR-103(SEQ ID NO:105), miR-130a (SEQ ID NO:112), miR-107 (SEQ ID NO:175), miR-132 (SEQ ID NO:159), miR-1244(SEQ ID NO:20), miR-140-5p (SEQ ID NO:160), miR-1256 (SEQ ID NO:195), miR-141 (SEQ ID NO:161), miR-125a-5p (SEQ ID NO:176), miR-142-5p (SEQ ID NO:43), miR-127-3p (SEQ ID NO:101), miR-146a (SEQ ID NO:21), miR-142-3p (SEQ ID NO:38), miR-146b-5p (SEQ ID NO:24), miR-144# (SEQ ID NO:69), miR-148a (SEQ ID NO:91), miR-148b# (SEQ ID NO:128), miR-150 (SEQ ID NO:27), miR-154# (SEQ ID NO:139), miR-152 (SEQ ID NO:130), miR-15b (SEQ ID NO:56), miR-15a# (SEQ ID NO:64), miR-181a-2# (SEQ ID NO:177), miR-17 (SEQ ID NO:162), miR-190 (SEQ ID NO:63), miR-185 (SEQ ID NO:163), miR-196b (SEQ ID NO:140), miR-19a (SEQ ID NO:74), miR-19b-1# (SEQ ID NO:178), miR-21 (SEQ ID NO:51), miR-200c (SEQ ID NO:179), miR-21# (SEQ ID NO:141), miR-20a# (SEQ ID NO:75), miR-222 (SEQ ID NO:16), miR-24-2# (SEQ ID NO:120), miR-26a-2# (SEQ ID NO:82), miR-26a (SEQ ID NO:70), miR-30a-5p (SEQ ID NO:164), miR-27b# (SEQ ID NO:180), miR-30d (SEQ ID NO:165), miR-28-5p (SEQ ID NO:90), miR-324-3p (SEQ ID NO:107), miR-299-5p (SEQ ID NO:196), miR-335 (SEQ ID NO:119), miR-29b (SEQ ID NO:125), miR-345 (SEQ ID NO:18), miR-301a (SEQ ID NO:67), miR-34a (SEQ ID NO:166), miR-30b (SEQ ID NO:42), miR-362-3p (SEQ ID NO:96), miR-30c (SEQ ID NO:52), miR-378 (SEQ ID NO:167), miR-331-3p (SEQ ID NO:181), miR-425 (SEQ ID NO:168), miR-339-5p (SEQ ID NO:182), miR-429 (SEQ ID NO:169), miR-340# (SEQ ID NO:127), miR-523 (SEQ ID NO:171), miR-362-5p (SEQ ID NO:183), miR-551b# (SEQ ID NO:172), miR-370 (SEQ ID NO:184), miR-579 (SEQ ID NO:170), miR-374a (SEQ ID NO:68), miR-590-5p (SEQ ID NO:104), miR-374b (SEQ ID NO:185), miR-598 (SEQ ID NO:110), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO:190), miR-766 (SEQ ID NO:191), miR-9 (SEQ ID NO: 192), miR-98 (SEQ ID NO:193), miR-99b (SEQ ID NO:122), and combinations thereof.

7B. The method of embodiment 1B or embodiment 2B, wherein the one, two, three, four, five, six, seven or more microRNA are selected from the group consisting of let-7a (SEQ ID NO:173), let-7a# (SEQ ID NO:155), miR-106a (SEQ ID NO:156), let-7e (SEQ ID NO:93), miR-122 (SEQ ID NO:22), let-7f (SEQ ID NO:174), miR-1227 (SEQ ID NO:157), let-7g (SEQ ID NO:72), miR-130a (SEQ ID NO:112), miR-107 (SEQ ID NO:175), miR-1244(SEQ ID NO:20), miR-140-5p (SEQ ID NO:160), miR-1256 (SEQ ID NO:195), miR-141 (SEQ ID NO:161), miR-125a-5p (SEQ ID NO:176), miR-142-5p (SEQ ID NO:43), miR-127-3p (SEQ ID NO:101), miR-146a (SEQ ID NO:21), miR-146b-5p (SEQ ID NO:24), miR-144# (SEQ ID NO:69), miR-148a (SEQ ID NO:91), miR-148b# (SEQ ID NO:128), miR-154# (SEQ ID NO:139), miR-152 (SEQ ID NO:130), miR-15b (SEQ ID NO:56), miR-15a# (SEQ ID NO:64), miR-181a-2# (SEQ ID NO:177), miR-17 (SEQ ID NO:162), miR-190 (SEQ ID NO:63), miR-185 (SEQ ID NO:163), miR-196b (SEQ ID NO:140), miR-19a (SEQ ID NO:74), miR-19b-1# (SEQ ID NO:178), miR-200c (SEQ ID NO:179), miR-21# (SEQ ID NO:141), miR-20a# (SEQ ID NO:75), miR-222 (SEQ ID NO:16), miR-24-2# (SEQ ID NO:120), miR-26a-2# (SEQ ID NO:82), miR-30a-5p (SEQ ID NO:164), miR-27b# (SEQ ID NO:180), miR-30d (SEQ ID NO:165), miR-28-5p (SEQ ID NO:90), miR-299-5p (SEQ ID NO:196), miR-335 (SEQ ID NO:119), miR-345 (SEQ ID NO:18), miR-301a (SEQ ID NO:67), miR-34a (SEQ ID NO:166), miR-362-3p (SEQ ID NO:96), miR-378 (SEQ ID NO:167), miR-331-3p (SEQ ID NO:181), miR-425 (SEQ ID NO:168), miR-339-5p (SEQ ID NO:182), miR-429 (SEQ ID NO:169), miR-340# (SEQ ID NO:127), miR-523 (SEQ ID NO:171), miR-362-5p (SEQ ID NO:183), miR-551b# (SEQ ID NO:172), miR-370 (SEQ ID NO:184), miR-579 (SEQ ID NO:170), miR-374a (SEQ ID NO:68), miR-590-5p (SEQ ID NO:104), miR-374b (SEQ ID NO:185), miR-598 (SEQ ID NO:110), miR-379 (SEQ ID NO:186), miR-411 (SEQ ID NO:124), miR-411 (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO:190), miR-766 (SEQ ID NO:191), miR-9 (SEQ ID NO:192), miR-98 (SEQ ID NO:193), miR-99b (SEQ ID NO:122), and combinations thereof.

8B. The method of embodiment 1B or embodiment 2B, wherein the one, two, three, four, five, six, seven or more microRNA are selected from the group consisting of let-7a (SEQ ID NO:173), miR-132 (SEQ ID NO:159), let-7d (SEQ ID NO:45), miR-148a (SEQ ID NO:91), let-7e (SEQ ID NO:93), miR-152 (SEQ ID NO:130), let-7f (SEQ ID NO:174), miR-17 (SEQ ID NO:162), miR-103 (SEQ ID NO:105), miR-185 (SEQ ID NO: 163), miR-1256 (SEQ ID NO:195), miR-21 (SEQ ID NO:51), miR-142-3p (SEQ ID NO: 38), miR-222 (SEQ ID NO:16), miR-144# (SEQ ID NO:69), miR-345 (SEQ ID NO:18), miR-148b# (SEQ ID NO:128), miR-34a (SEQ ID NO:166), miR-154# (SEQ ID NO:139), miR-523 (SEQ ID NO:171), miR-15b (SEQ ID NO:56), miR-551b# (SEQ ID NO:172), miR-181a-2# (SEQ ID NO:177), miR-590-5p (SEQ ID NO:104), miR-190 (SEQ ID NO: 63), miR-20a# (SEQ ID NO:75), miR-24-2# (SEQ ID NO:120), miR-26a (SEQ ID NO:70), miR-28-5p (SEQ ID NO:90), miR-299-5p (SEQ ID NO:196), miR-30b (SEQ ID NO:42), miR-30c (SEQ ID NO:52), miR-331-3p (SEQ ID NO:181), miR-340# (SEQ ID NO:127), miR-362-5p (SEQ ID NO:183), miR-374a (SEQ ID NO:68), miR-379 (SEQ ID NO:186), miR-411 (SEQ ID NO:124), miR-411# (SEQ ID NO:147), miR-454 (SEQ ID NO:187), miR-520a-3p (SEQ ID NO:188), miR-668 (SEQ ID NO:194), miR-758 (SEQ ID NO:190), and combinations thereof.

9B. The method of embodiment 1B or embodiment 2B, wherein the one, two, three, four, five, six, seven or more microRNA are selected from the group consisting of let-7a (SEQ ID NO:173), miR-148a (SEQ ID NO:91), let-7e (SEQ ID NO:93), miR-152 (SEQ ID NO:130), let-7f (SEQ ID NO:174), miR-17 (SEQ ID NO:162), miR-185 (SEQ ID NO:163), miR-1256 (SEQ ID NO:195), miR-222 (SEQ ID NO:16), miR-144# (SEQ ID NO:69), miR-345 (SEQ ID NO:18), miR-148b# (SEQ ID NO:128), miR-34a (SEQ ID NO: 166), miR-154# (SEQ ID NO:139), miR-523 (SEQ ID NO:171), miR-15b (SEQ ID NO: 56), miR-551b# (SEQ ID NO:172), miR-181a-2# (SEQ ID NO:177), miR-590-5p (SEQ ID NO:104), miR-190 (SEQ ID NO:63), miR-20a# (SEQ ID NO:75), miR-24-2# (SEQ ID NO:120), miR-28-5p (SEQ ID NO:90), miR-299-5p (SEQ ID NO:196), miR-331-3p (SEQ ID NO:181), miR-340# (SEQ ID NO:127), miR-362-5p (SEQ ID NO:183), miR-374a (SEQ ID NO:68), miR-379 (SEQ ID NO:186), miR-411 (SEQ ID NO:124), miR-411 (SEQ ID NO:147), miR-454 (SEQ ID NO:187), miR-520a-3p (SEQ ID NO:188), miR-668 (SEQ ID NO:194), miR-758 (SEQ ID NO:190), and combinations thereof.

10B. The method of embodiment 1B or embodiment 2B, wherein the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), and combinations thereof.

11B The method of embodiment 1B or embodiment 2B, wherein the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), and combinations thereof.

12B. The method of embodiment 1B or embodiment 2B, wherein the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), and combinations thereof.

13B. The method of embodiment 1B or embodiment 2B, wherein the one, two, three, four, five, six, seven or more microRNA are selected from the group consisting of let-7a (SEQ ID NO:173), let-7e (SEQ ID NO:93), let-7f (SEQ ID NO:174), let-7g (SEQ ID NO:72), miR-107 (SEQ ID NO:175), miR-1244 (SEQ ID NO:20), miR-1256 (SEQ ID NO: 195), miR-127-3p (SEQ ID NO:101), miR-144# (SEQ ID NO:69), miR-148b# (SEQ ID NO:128), miR-154# (SEQ ID NO:139), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-27b# (SEQ ID NO:180), miR-28-5p (SEQ ID NO:90), miR-299-5p (SEQ ID NO:196), miR-301a (SEQ ID NO:67), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-411 (SEQ ID NO:124), miR-411# (SEQ ID NO:147), miR-454 (SEQ ID NO:187), miR-520a-3p (SEQ ID NO:188), miR-539 (SEQ ID NO:189), miR-543 (SEQ ID NO:133), miR-548J (SEQ ID NO:148), miR-664 (SEQ ID NO:152), miR-668 (SEQ ID NO:194), miR-758 (SEQ ID NO: 190), miR-766 (SEQ ID NO:191), miR-9 (SEQ ID NO:192), miR-98 (SEQ ID NO:193), miR-99b (SEQ ID NO:122), and combinations thereof.

14B. The method of embodiment 1B or embodiment 2B, wherein the one, two, three, four, five, six, seven or more microRNA are selected from the group consisting of let-7d (SEQ ID NO:45), miR-103 (SEQ ID NO:105), miR-125a-5p (SEQ ID NO:176), miR-142-3p (SEQ ID NO:38), miR-26a (SEQ ID NO:70), miR-29b (SEQ ID NO:125), miR-30b (SEQ ID NO:42), miR-30c (SEQ ID NO:52), miR-379 (SEQ ID NO:186) and combinations thereof.

15B. The method of embodiment 1B or embodiment 2B, wherein the one, two, three, four, five, six, seven or more microRNA are selected from the group consisting of miR-18a (SEQ ID NO:39), miR-26b (SEQ ID NO:40), miR-106b (SEQ ID NO:41), miR-29c (SEQ ID NO:44), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-148b (SEQ ID NO:53), miR-27b (SEQ ID NO:54), miR-15b# (SEQ ID NO:55), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-27a (SEQ ID NO:65), miR-181a (SEQ ID NO:66), miR-320B (SEQ ID NO:71), miR-324-5p (SEQ ID NO:73), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-106b# (SEQ ID NO:92), miR-25 (SEQ ID NO:94), miR-656 (SEQ ID NO: 95), miR-340 (SEQ ID NO:97), miR-451 (SEQ ID NO:98), miR-423-5p (SEQ ID NO:99), miR-652 (SEQ ID NO:100), miR-495 (SEQ ID NO:102), miR-328 (SEQ ID NO:103), miR-19b (SEQ ID NO:106), miR-145# (SEQ ID NO:108), miR-199a-3p (SEQ ID NO: 109), miR-151-5P (SEQ ID NO:111), miR-502-3p (SEQ ID NO:113), miR-136# (SEQ ID NO:114), miR-194 (SEQ ID NO:115), miR-221 (SEQ ID NO:116), miR-22# (SEQ ID NO: 117), miR-93 (SEQ ID NO:118), miR-130b (SEQ ID NO:121), miR-195 (SEQ ID NO: 123), miR-576-3p (SEQ ID NO:126), miR-212 (SEQ ID NO:129), miR-143 (SEQ ID NO: 131), dme-miR-7 (SEQ ID NO:132), miR-30d (SEQ ID NO:134), miR-213 (SEQ ID NO: 135), miR-126# (SEQ ID NO:136), miR-1197 (SEQ ID NO:137), miR-1255B (SEQ ID NO: 138), miR-335# (SEQ ID NO:142), miR-33a# (SEQ ID NO:143), miR-374a# (SEQ ID NO: 144), miR-381 (SEQ ID NO:145), miR-409-5p (SEQ ID NO:146), miR-551b (SEQ ID NO: 149), miR-616 (SEQ ID NO:150), miR-638 (SEQ ID NO:151), miR-889 (SEQ ID NO: 153), rno-miR-29c# (SEQ ID NO:154) and combinations thereof.

16B. The method of embodiment 1B or embodiment 2B, wherein the one, two, three, four, five, six, seven or more microRNA are selected from the group consisting of let-7a (SEQ ID NO:173), let-7d (SEQ ID NO:45), let-7e (SEQ ID NO:93), let-7f (SEQ ID NO:174), miR-103 (SEQ ID NO:105), miR-142-3p (SEQ ID NO:38), miR-144# (SEQ ID NO:69), miR-148b# (SEQ ID NO:128), miR-154# (SEQ ID NO:139), miR-15b (SEQ ID NO:56), miR-181a-2# (SEQ ID NO:177), miR-190 (SEQ ID NO:63), miR-20a# (SEQ ID NO:75), miR-24-2# (SEQ ID NO:120), miR-26a (SEQ ID NO:70), miR-28-5p (SEQ ID NO:90), miR-30b (SEQ ID NO:42), miR-30c (SEQ ID NO:52), miR-331-3p (SEQ ID NO: 181), miR-340# (SEQ ID NO:127), miR-362-5p (SEQ ID NO:183), miR-374a (SEQ ID NO:68), miR-379 (SEQ ID NO:186), miR-411 (SEQ ID NO:124), miR-411# (SEQ ID NO: 147), miR-454 (SEQ ID NO:187), miR-520a-3p (SEQ ID NO:188), miR-758 (SEQ ID NO: 190), miR-1256 (SEQ ID NO:195), miR-299-5p (SEQ ID NO:196), miR-668 (SEQ ID NO: 194), and combinations thereof.

17B. The method of embodiment 1B or embodiment 2B, wherein the one, two, three, four, five, six, seven or more microRNA are selected from the group consisting of let-7g (SEQ ID NO:72), miR-107 (SEQ ID NO:175), miR-1244 (SEQ ID NO:20), miR-125a-5p (SEQ ID NO:176), miR-127-3p (SEQ ID NO:101), miR-196b (SEQ ID NO:140), miR-19b-1# (SEQ ID NO:178), miR-200c (SEQ ID NO:179), miR-27b# (SEQ ID NO: 180), miR-29b (SEQ ID NO:125), miR-301a (SEQ ID NO:67), miR-339-5p (SEQ ID NO: 182), miR-370 (SEQ ID NO:184), miR-374b (SEQ ID NO:185), miR-539 (SEQ ID NO:189), miR-543 (SEQ ID NO:133), miR-548J (SEQ ID NO:148), miR-664 (SEQ ID NO: 152), miR-766 (SEQ ID NO:191), miR-9 (SEQ ID NO:192), miR-98 (SEQ ID NO:193), miR-99b (SEQ ID NO:122), and combinations thereof.

18B. The method of any one of embodiments 1B-17B, wherein the level or expression pattern of at least 2 different microRNAs is detected.

19B. The method of any one of embodiments 1B-17B, wherein the level or expression pattern of at least 10 different microRNA is detected.

20B. The method of any one of embodiments 1B-17B, wherein the level or expression pattern of at least 20 different microRNA is detected.

21B. The method of any one of embodiments 1B-4B, wherein the presence or an increased level of one, two, three, four, five, six, seven or more microRNA is detected, relative to a predetermined criterion, is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-130a (SEQ ID NO:112), miR-142-5p (SEQ ID NO:43), miR-148a (SEQ ID NO:91), miR-152 (SEQ ID NO:130), miR-15a# (SEQ ID NO:64), miR-19a (SEQ ID NO:74), miR-21 (SEQ ID NO:51), miR-21# (SEQ ID NO:141), miR-26a-2 (SEQ ID NO:82), miR-324-3p (SEQ ID NO:107), miR-335 (SEQ ID NO:119), miR-362-3p (SEQ ID NO:96, miR-590-5p (SEQ ID NO:104), miR-598 (SEQ ID NO:110), let-7a# (SEQ ID NO:155), miR-106a (SEQ ID NO:156), miR-1227 (SEQ ID NO:157), miR-128 (SEQ ID NO:158), miR-132 (SEQ ID NO:159), miR-140-5p (SEQ ID NO:160), miR-141 (SEQ ID NO:161), miR-17 (SEQ ID NO:162), miR-185 (SEQ ID NO:163), miR-30a-5p (SEQ ID NO:164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), and combinations thereof.

22B. The method of any one of embodiments 1B-4B, wherein the presence or an increased level of one, two, three, four, five, six, seven or more microRNA is detected, relative to a predetermined criterion, is indicative of a diagnosis of IPF, and the one or more microRNAs is selected from the group consisting of miR-222 (SEQ ID NO:16), miR-345 (SEQ ID NO:18), miR-146a (SEQ ID NO:21), miR-122 (SEQ ID NO:22), miR-146b-5p (SEQ ID NO:24), miR-1300 (SEQ ID NO:25), miR-150 (SEQ ID NO:27), miR-130a (SEQ ID NO:112), miR-142-5p (SEQ ID NO:43), miR-148a (SEQ ID NO:91), miR-152 (SEQ ID NO:130), miR-15a# (SEQ ID NO:64), miR-19a (SEQ ID NO:74), miR-21 (SEQ ID NO: 51), miR-21# (SEQ ID NO:141), miR-26a-2# (SEQ ID NO:82), miR-324-3p (SEQ ID NO: 107), miR-335 (SEQ ID NO:119), miR-362-3p (SEQ ID NO:96, miR-590-5p (SEQ ID NO: 104), miR-598 (SEQ ID NO:110), let-7a# (SEQ ID NO:155), miR-106a (SEQ ID NO:156), miR-1227 (SEQ ID NO:157), miR-128 (SEQ ID NO:158), miR-132 (SEQ ID NO:159), miR-140-5p (SEQ ID NO:160), miR-141 (SEQ ID NO:161), miR-17 (SEQ ID NO:162), miR-185 (SEQ ID NO:163), miR-30a-5p (SEQ ID NO:164), miR-30d (SEQ ID NO:165), miR-34a (SEQ ID NO:166), miR-378 (SEQ ID NO:167), miR-425 (SEQ ID NO:168), miR-429 (SEQ ID NO:169), miR-579 (SEQ ID NO:170), miR-523 (SEQ ID NO:171), miR-551b# (SEQ ID NO:172), and combinations thereof.

23B. The method of any one of embodiments 1B-4B, wherein the presence or an increased level of one, two, three, four, five, six, seven or more microRNA is detected, relative to a predetermined criterion, is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11); miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21) and combinations thereof.

24B. The method of any one of embodiments 1B-4B, wherein the presence or an increased level of one, two, three, four, five, six, seven or more microRNA is detected, relative to a predetermined criterion, is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11); miR-193b (SEQ ID NO:12), miR-34a# (SEQ ID NO:13), miR-548a-3p (SEQ ID NO:14) and combinations thereof.

25B. The method of any one of embodiments 1B-4B, wherein the presence or an increased level of one, two, three, four, five, six, seven or more microRNA is detected, relative to a predetermined criterion, is indicative of a diagnosis of IPF, and the one or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11) and combinations thereof.

26B. The method of any one of embodiments 1B-25B, wherein the absence or a decreased level of one, two, three, four, five, six, seven or more microRNA is detected, relative to a predetermined criterion, indicative of a diagnosis of IPF, and the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2 (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof.

27B. The method of any one of embodiments 1B-25B, wherein the absence or a decreased level of one, two, three, four, five, six, seven or more microRNA is detected, relative to a predetermined criterion, indicative of a diagnosis of IPF, and the one or more microRNAs is selected from the group consisting of miR-1244 (SEQ ID NO:20), miR-142-3p (SEQ ID NO:38), miR-30b (SEQ ID NO:42), let-7d (SEQ ID NO:45), miR-30c (SEQ ID NO:52), miR-15b (SEQ ID NO:56), miR-190 (SEQ ID NO:63), miR-301a (SEQ ID NO:67), miR-374a (SEQ ID NO:68), miR-144# (SEQ ID NO:69), miR-26a (SEQ ID NO:70), let-7g (SEQ ID NO:72), miR-20a# (SEQ ID NO:75), miR-28-5p (SEQ ID NO:90), let-7e (SEQ ID NO:93), miR-127-3p (SEQ ID NO:101), miR-103 (SEQ ID NO:105), miR-24-2# (SEQ ID NO:120), miR-99b (SEQ ID NO:122), miR-411 (SEQ ID NO:124), miR-29b (SEQ ID NO:125), miR-340# (SEQ ID NO:127), miR-148b# (SEQ ID NO:128), miR-543 (SEQ ID NO:133), miR-154# (SEQ ID NO:139), miR-196b (SEQ ID NO:140), miR-411# (SEQ ID NO:147), miR-548J (SEQ ID NO:148), miR-664 (SEQ ID NO:152), let-7a (SEQ ID NO: 173), let-7f (SEQ ID NO:174), miR-107 (SEQ ID NO:175), miR-125a-5p (SEQ ID NO: 176), miR-181a-2# (SEQ ID NO:177), miR-19b-1# (SEQ ID NO:178), miR-200c (SEQ ID NO:179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO:191), miR-9 (SEQ ID NO:192), miR-98 (SEQ ID NO:193), miR-668 (SEQ ID NO: 194), miR-1256 (SEQ ID NO:195), miR-299-5p (SEQ ID NO:196), and combinations thereof.

28B. The method of any one of embodiments 1B-25B, wherein the absence or a decreased level of one, two, three, four, five, six, seven or more microRNA is detected, relative to a predetermined criterion, indicative of a diagnosis of IPF, and the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g# (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88),) and combinations thereof.

29B. The method of any one of embodiments 1B-25B, wherein the absence or a decreased level of one, two, three, four, five, six, seven or more microRNA is detected, relative to a predetermined criterion, indicative of a diagnosis of IPF, and the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61) and combinations thereof.

30B. The method of any one of embodiments 1B-25B, wherein the absence or a decreased level of one, two, three, four, five, six, seven or more microRNA is detected, relative to a predetermined criterion, indicative of a diagnosis of IPF, and the one or more microRNAs is selected from the group consisting of miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), and combinations thereof.

31B. The method of any of embodiments 1B-30B wherein the sample is selected from the group consisting of whole blood, serum, plasma, exosomes and isolated microvesicles.

32B. The method of any one of embodiments 1B-31B, wherein the method further comprises administering a therapeutic agent to the subject.

33B. A method of treating a human subject diagnosed with idiopathic pulmonary fibrosis (IPF) according to any of methods 1B-30B comprising administering a therapeutic agent to the subject to treat IPF.

34B. A method of treating a human subject identified as having idiopathic pulmonary fibrosis (IPF) or at risk of IPF based on an abnormal level of one, two, three, four, five, six, seven or more IPF-associated microRNAs in a blood sample of the subject, comprising administering a therapeutic agent to the subject to treat IPF.

35B. The method of embodiment 33B or embodiment 34B, wherein the one, two, three, four, five, six, seven or more microRNAs is selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:11l), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a# (SEQ ID NO: 155), miR-106a (SEQ ID NO:156), miR-1227 (SEQ ID NO:157), miR-128 (SEQ ID NO: 158), miR-132 (SEQ ID NO:159), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO: 179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO: 191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), or combinations thereof.

36B. The method of embodiment 33B or embodiment 34B, wherein the level or expression pattern of at least 2 different microRNAs is detected.

37B. The method of embodiment 33B or embodiment 34B, wherein the level or expression pattern of at least 10 different microRNA is detected.

38B. The method of embodiment 33B or embodiment 34B, wherein the level or expression pattern of at least 20 different microRNA is detected.

39. The method of embodiment 34B, wherein the sample is selected from the group consisting of whole blood, serum, plasma, exosomes and isolated micro vesicles.

40B. The method of any of embodiments 32B-39B, wherein the therapeutic agent is an oligonucleotide that decreases the activity or level of expression of one, two, three, four, five, six, seven or more of the microRNA in the subject.

41B. The method of any of embodiments 32B-39B, wherein the therapeutic agent is an oligonucleotide that increases the activity or level of expression of one, two, three, four, five, six, seven or more of the microRNA in the subject.

42B. The method of any of embodiments 32B-39B, wherein the therapeutic agent is an anti-fibrotic agent.

43B. The method of embodiment 42B, wherein the anti-fibrotic agent is pirfenidone.

44B. The method of any of embodiments 32B-39B, wherein the therapeutic agent is selected from the group consisting of steroids (including but not limited to prednisolone), cytotoxic agents (including but not limited to azathioprine and cyclophosphamide), bardoxolone, LPA agonists (including but not limited to AM152); Torisel (temsirolimus); PI3K inhibitors; pentraxin or serum amyloid P (including but not limited to Pentraxin-2 (PTX-2 or PRM-151)); MEK inhibitors (including but not limited to ARRY-162 and ARRY-300); p38 inhibitors; PAI-1 inhibitors (including but not limited to Tiplaxtinin); agents that reduce the activity of transforming growth factor-beta (TGF-β) (including but not limited to GC-1008 (Genzyme/MedImmune); lerdelimumab (CAT-152; Trabio, Cambridge Antibody); metelimumab (CAT-192, Cambridge Antibody,); LY-2157299 (Eli Lilly); ACU-HTR-028 (Opko Health)) including antibodies that target one or more TGF-β isoforms, inhibitors of TGF-β receptor kinases TGFBR1 (ALK5) and TGFBR2, and modulators of post-receptor signaling pathways; chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610; PS-433540; S-0139; sitaxentan sodium; TBC-3711; zibotentan); agents that reduce the activity of connective tissue growth factor (CTGF) (including but not limited to FG-3019, FibroGen), and including other CTGF-neutralizing antibodies; matrix metalloproteinase (MMP) inhibitors (including but not limited to MMPI-12, PUP-1 and tigapotide triflutate); agents that reduce the activity of epidermal growth factor receptor (EGFR) including but not limed to erlotinib, gefitinib, BMS-690514, cetuximab, antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Ra) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573 or rapamycin); agents that reduce levels of copper such as tetrathiomolybdate; agents that reduce oxidative stress including N-acetyl cysteine and tetrathiomolybdate; and interferon gamma, inhibitors of phosphodiesterase 4 (PDE4) (including but not limited to Roflumilast); inhibitors of phosphodiesterase 5 (PDE5) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton), compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil, P-1894B, and safironil) and peroxisome proliferator-activated receptor (PPAR)-gamma agonists (including but not limited to pioglitazone and rosiglitazone), and combinations thereof.

45B. A kit to be used in the diagnosis of subjects having idiopathic pulmonary fibrosis (IPF) comprising one, two, three, four, five, six, seven or more probes that specifically hybridize to, or primers that specifically amplify, one or more microRNAs selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a# (SEQ ID NO: 155), miR-106a (SEQ ID NO:156), miR-1227 (SEQ ID NO:157), miR-128 (SEQ ID NO: 158), miR-132 (SEQ ID NO:159), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO: 179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO: 191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), and combinations thereof.

46B. A diagnostic test system adapted for performing any of the methods of embodiments 1B-31B.

47B. The diagnostic test system of embodiment 46B comprising means for obtaining test results comprising the activity or level of one, two, three, four, five, six, seven or more microRNA correlated with a diagnosis of idiopathic pulmonary fibrosis (IPF) in a blood sample of the subject; means for collecting and tracking test results for one or more individual blood sample; means for comparing the activity or level of one or more microRNA to a predetermined criterion; and means for reporting whether the activity or level of the one or more microRNA meets or exceeds the predetermined criterion

48B. A computer program product comprising computer-executable instructions embodied in a computer-readable medium for performing the steps of any of the methods of embodiments 1B-31B.

The invention is further described in the following Examples. The following Examples serve only to illustrate the invention and are not intended to limit the scope of the invention in any way.

### EXAMPLES

### Example 1

### IPF Patients were Determined to have a Unique miRNA Profile Compared to Healthy Controls

Materials and Methods:

Plasma samples were obtained from placebo-treated Caucasian male IPF patients. The design of this trial including patient inclusion/exclusion criteria and treatment have been previously published (King et al 2009). Plasma samples for demo graphically matched healthy control subjects with associated medical histories and medication use were obtained commercially. Plasma samples from IPF patients were collected in vials containing heparin as the anticoagulant, while sample from control subjects were collected in vials containing EDTA. All samples were obtained under appropriate written Informed Consent. Histories were reviewed for the following additional criteria: not a current smoker (both groups), lack of pulmonary or other significant disease (healthy control group), no recent use of prednisolone or other drugs used off label in the treatment of IPF (healthy control group), no use of prednisolone or other drugs for the treatment of IPF within 28 days prior to sample collection (IPF group). Summary demographics for IPF patients and healthy controls are shown in Table 1.

TABLE 1

| | | | INSPIRE |
|---|---|---|---|
| | Healthy | IPF | Placebo |
| Total # | 12 | 24 | 275 |
| Ethnic origin White | 100% | 100% | 95% |
| Caucasian male | 100% | 100% | 68% |
| Age (year)^{a} | 58.2 (5.9) | 67.1 (8.0) | 65.9 (7.9) |
| Weight (kg)^{b} | 84.3 (11.8) | 90.5 (17.4) | 86.6 (18.1) |
| Smoking status | | | |
| Never smoked | 17% | 17% | 31% |
| Previous smoker | 83% | 83% | 64% |
| Current smoker | 0 | 0 | 5% |
| FVC | | 78.2 (11.3) | 73.1 (13.4) |
| DLco | | 47.0 (6.7) | 47.3 (9.3) |
| Dyspnea (total score of shortness of breath | | 32.8 (24.5) | 35.0 (22.7) |
| questionnaires) | | | |
| Quality of life (total score of St George's respiratory | | 41.5 (20.0) | 42.4 (18.2) |
| questionnaire) | | | |
| Distance of 6-minute walk | | 404.6 (90.6) | 392.8 (112.9) |
| Use of supplemental oxygen | | 4% | 15% |

^{a}p-value of t-test between controls and IPF patient is 0.003
^{b}p-value of t-test between controls and IPF patient is 0.1

Samples from IPF patients (n=24), healthy controls (n=12), and appropriate technical replicates (n=8 from IPF patients and n=4 from healthy controls) were deheparanized and grouped into two batches each for RNA isolation by standard methods. Total RNA was extracted by standard methods. Isolated RNA was reverse transcribed and preamplified using the Applied Biosystems Megaplex RT and Preamplification Human Primer Pools according to manufacturer's protocols.

miRNAs were profiled by real-time PCR using the TaqMan Human miRNA Array set v3.0 (Cards A+B) according to manufacturer's protocols.

microRNAs with Ct values less than 35 were selected for data analysis. microRNAs for normalization were selected by a mean centering method (Wylie et al, BMC Research Notes, 4:555, 2011). Normalization and all subsequent analyses were performed in Partek Genomics Suite (Partek, St. Louis, Mo.). Differentially expressed miRNAs were identified by ANOVA with correction for multiple comparisons. Sequences detected in <50% of samples in both the IPF and control groups were excluded. miRNAs present in <50% of samples from one or the other group were evaluated as potentially disease-status specific sequences.

miRNAs identified as having increased presentation in IPF patient plasma relative to a predetermined criterion are set forth below in Table 2.

TABLE 2

| | | Differential | SEQ ID |
|---|---|---|---|
| microRNA ID | Sequence Data | Regulation | NO. |
| hsa-miR-155 | UUAAUGCUAAUCGUGAUAGGGGU | IPF UP vs Control | 1 |
| hsa-miR-767-3p | UCUGCUCAUACCCCAUGGUUUCU | IPF UP vs Control | 2 |
| hsa-miR-1303 | UUUAGAGACGGGGUCUUGCUCU | IPF UP vs Control | 3 |
| hsa-miR-574-3p | CACGCUCAUGCACACACCCACA | IPF UP vs Control | 4 |
| hsa-miR-10b# | ACAGAUUCGAUUCUAGGGGAAU | IPF UP vs Control | 5 |
| hsa-miR-875-5p | UAUACCUCAGUUUUAUCAGGUG | IPF UP vs Control | 6 |
| hsa-miR-29a | UAGCACCAUCUGAAAUCGGUUA | IPF UP vs Control | 7 |
| hsa-miR-375 | UUUGUUCGUUCGGCUCGCGUGA | IPF UP vs Control | 8 |
| hsa-miR-342-3p | UCUCACACAGAAAUCGCACCCGU | IPF UP vs Control | 9 |
| hsa-miR-197 | UUCACCACCUUCUCCACCCAGC | IPF UP vs Control | 10 |
| hsa-miR-663B | GGUGGCCCGGCCGUGCCUGAGG | IPF UP vs Control | 11 |
| hsa-miR-193b | AACUGGCCCUCAAAGUCCCGCU | IPF UP vs Control | 12 |
| hsa-miR-34a# | CAAUCAGCAAGUAUACUGCCCU | IPF UP vs Control | 13 |
| hsa-miR-548a-3p | CAAAACUGGCAAUUACUUUUGC | IPF UP vs Control | 14 |
| hsa-miR-338-5P | AACAAUAUCCUGGUGCUGAGUG | IPF UP vs Control | 15 |
| hsa-miR-222 | AGCUACAUCUGGCUACUGGGU | IPF UP vs Control | 16 |
| hsa-miR-520D-3P | AAAGUGCUUCUCUUUGGUGGGU | IPF UP vs Control | 17 |
| hsa-miR-345 | GCUGACUCCUAGUCCAGGGCUC | IPF UP vs Control | 18 |
| hsa-miR-99b# | CAAGCUCGUGUCUGUGGGUCCG | IPF UP vs Control | 19 |
| hsa-miR-1244 | AAGUAGUUGGUUUGUAUGAGAUGGUU | IPF UP vs Control | 20 |
| hsa-miR-146a | UGAGAACUGAAUUCCAUGGGUU | IPF UP vs Control | 21 |
| hsa-miR-122 | UGGAGUGUGACAAUGGUGUUUG | IPF UP vs Control | 22 |
| hsa-miR-206 | UGGAAUGUAAGGAAGUGUGUGG | IPF UP vs Control | 23 |
| hsa-miR-146b-5p | UGAGAACUGAAUUCCAUAGGCU | IPF UP vs Control | 24 |
| hsa-miR-1300 | UUGAGAAGGAGGCUGCUG | IPF UP vs Control | 25 |
| hsa-miR-28-3p | CACUAGAUUGUGAGCUCCUGGA | IPF UP vs Control | 26 |
| hsa-miR-150 | UCUCCCAACCCUUGUACCAGUG | IPF UP vs Control | 27 |
| hsa-miR-202 | AGAGGUAUAGGGCAUGGGAA | IPF UP vs Control | 28 |
| hsa-miR-636 | UGUGCUUGCUCGUCCCGCCCGCA | IPF UP vs Control | 29 |
| hsa-miR-27a# | AGGGCUUAGCUGCUUGUGAGCA | IPF UP vs Control | 30 |
| hsa-miR-323-3p | CACAUUACACGGUCGACCUCU | IPF UP vs Control | 31 |
| hsa-miR-520c-3p | AAAGUGCUUCCUUUUAGAGGGU | IPF UP vs Control | 32 |
| hsa-miR-191 | CAACGGAAUCCCAAAAGCAGCUG | IPF UP vs Control | 33 |
| hsa-miR-1290 | UGGAUUUUUGGAUCAGGGA | IPF UP vs Control | 34 |
| hsa-miR-572 | GUCCGCUCGGCGGUGGCCCA | IPF UP vs Control | 35 |
| hsa-miR-886-3p | CGCGGGUGCUUACUGACCCUU | IPF UP vs Control | 36 |
| hsa-miR-320 | AAAAGCUGGGUUGAGAGGGCGA | IPF UP vs Control | 37 |

miRNAs identified as having decreased presentation in IPF patient plasma relative to a predetermined criterion are set forth below in Table 3.

TABLE 3

| | | Differential | SEQ ID |
|---|---|---|---|
| microRNA ID | Sequence Data | Regulation | NO. |
| hsa-miR-142-3p | UGUAGUGUUUCCUACUUUAUGGA | IPF DOWN vs Control | 38 |
| hsa-miR-18a | UAAGGUGCAUCUAGUGCAGAUAG | IPF DOWN vs Control | 39 |
| hsa-miR-26b | UUCAAGUAAUUCAGGAUAGGU | IPF DOWN vs Control | 40 |
| hsa-miR-106b | UAAAGUGCUGACAGUGCAGAU | IPF DOWN vs Control | 41 |
| hsa-miR-30b | UGUAAACAUCCUACACUCAGCU | IPF DOWN vs Control | 42 |
| hsa-miR-142-5p | CAUAAAGUAGAAAGCACUACU | IPF DOWN vs Control | 43 |
| hsa-miR-29c | UAGCACCAUUUGAAAUCGGUUA | IPF DOWN vs Control | 44 |
| hsa-let-7d | AGAGGUAGUAGGUUGCAUAGUU | IPF DOWN vs Control | 45 |
| hsa-miR-144 | UACAGUAUAGAUGAUGUACU | IPF DOWN vs Control | 46 |
| hsa-miR-1260 | AUCCCACCUCUGCCACCA | IPF DOWN vs Control | 47 |
| hsa-miR-361-5p | UUAUCAGAAUCUCCAGGGGUAC | IPF DOWN vs Control | 48 |
| hsa-miR-520e | AAAGUGCUUCCUUUUUGAGGG | IPF DOWN vs Control | 49 |
| hsa-miR-660 | UACCCAUUGCAUAUCGGAGUUG | IPF DOWN vs Control | 50 |
| hsa-miR-21 | UAGCUUAUCAGACUGAUGUUGA | IPF DOWN vs Control | 51 |
| hsa-miR-30c | UGUAAACAUCCUACACUCUCAGC | IPF DOWN vs Control | 52 |
| hsa-miR-148b | UCAGUGCAUCACAGAACUUUGU | IPF DOWN vs Control | 53 |
| hsa-miR-27b | UUCACAGUGGCUAAGUUCUGC | IPF DOWN vs Control | 54 |
| hsa-miR-15b# | CGAAUCAUUAUUUGCUGCUCUA | IPF DOWN vs Control | 55 |
| hsa-miR-15b | UAGCAGCACAUCAUGGUUUACA | IPF DOWN vs Control | 56 |
| hsa-miR-16-1# | CCAGUAUUAACUGUGCUGCUGA | IPF DOWN vs Control | 57 |
| hsa-miR-17# | ACUGCAGUGAAGGCACUUGUAG | IPF DOWN vs Control | 58 |
| hsa-miR-22 | AAGCUGCCAGUUGAAGAACUGU | IPF DOWN vs Control | 59 |
| hsa-miR-32 | UAUUGCACAUUACUAAGUUGCA | IPF DOWN vs Control | 60 |
| hsa-miR-532-5p | CAUGCCUUGAGUGUAGGACCGU | IPF DOWN vs Control | 61 |
| hsa-miR-101 | UACAGUACUGUGAUAACUGAA | IPF DOWN vs Control | 62 |
| hsa-miR-190 | UGAUAUGUUUGAUAUAUUAGGU | IPF DOWN vs Control | 63 |
| hsa-miR-15a# | CAGGCCAUAUUGUGCUGCCUCA | IPF DOWN vs Control | 64 |
| hsa-miR-27a | UUCACAGUGGCUAAGUUCCGC | IPF DOWN vs Control | 65 |
| hsa-miR-181a | AACAUUCAACGCUGUCGGUGAGU | IPF DOWN vs Control | 66 |
| hsa-miR-301a | CAGUGCAAUAGUAUUGUCAAAGC | IPF DOWN vs Control | 67 |
| hsa-miR-374a | UUAUAAUACAACCUGAUAAGUG | IPF DOWN vs Control | 68 |
| hsa-miR-144# | GGAUAUCAUCAUAUACUGUAAG | IPF DOWN vs Control | 69 |
| hsa-miR-26a | UUCAAGUAAUCCAGGAUAGGCU | IPF DOWN vs Control | 70 |
| hsa-miR-320B | AAAAGCUGGGUUGAGAGGGCAA | IPF DOWN vs Control | 71 |
| hsa-let-7g | UGAGGUAGUAGUUUGUACAGUU | IPF DOWN vs Control | 72 |
| hsa-miR-324-5p | CGCAUCCCCUAGGGCAUUGGUGU | IPF DOWN vs Control | 73 |
| hsa-miR-19a | UGUGCAAAUCUAUGCAAAACUGA | IPF DOWN vs Control | 74 |
| hsa-miR-20a# | ACUGCAUUAUGAGCACUUAAAG | IPF DOWN vs Control | 75 |
| hsa-let-7b | UGAGGUAGUAGGUUGUGUGGUU | IPF DOWN vs Control | 76 |
| hsa-miR-422a | ACUGGACUUAGGGUCAGAAGGC | IPF DOWN vs Control | 77 |
| hsa-let-7f-2# | CUAUACAGUCUACUGUCUUUCC | IPF DOWN vs Control | 78 |
| hsa-let-7g# | CUGUACAGGCCACUGCCUUGC | IPF DOWN vs Control | 79 |
| hsa-miR-128a | UCACAGUGAACCGGUCUCUUU | IPF DOWN vs Control | 80 |
| hsa-miR-199a-5p | CCCAGUGUUCAGACUACCUGUUC | IPF DOWN vs Control | 81 |
| hsa-miR-26a-2# | CCUAUUCUUGAUUACUUGUUUC | IPF DOWN vs Control | 82 |
| hsa-miR-29a# | ACUGAUUUCUUUUGGUGUUCAG | IPF DOWN vs Control | 83 |
| hsa-miR-329 | AACACACCUGGUUAACCUCUUU | IPF DOWN vs Control | 84 |
| hsa-miR-337-5p | GAACGGCUUCAUACAGGAGUU | IPF DOWN vs Control | 85 |
| hsa-miR-369-3p | AAUAAUACAUGGUUGAUCUUU | IPF DOWN vs Control | 86 |
| hsa-miR-376a# | GUAGAUUCUCCUUCUAUGAGUA | IPF DOWN vs Control | 87 |
| hsa-miR-486-3p | CGGGGCAGCUCAGUACAGGAU | IPF DOWN vs Control | 88 |
| hsa-miR-20a | UAAAGUGCUUAUAGUGCAGGUAG | IPF DOWN vs Control | 89 |
| hsa-miR-28-5p | AAGGAGCUCACAGUCUAUUGAG | IPF DOWN vs Control | 90 |
| hsa-miR-148a | UCAGUGCACUACAGAACUUUGU | IPF DOWN vs Control | 91 |
| hsa-miR-106b# | CCGCACUGUGGGUACUUGCUGC | IPF DOWN vs Control | 92 |
| hsa-let-7e | UGAGGUAGGAGGUUGUAUAGUU | IPF DOWN vs Control | 93 |
| hsa-miR-25 | CAUUGCACUUGUCUCGGUCUGA | IPF DOWN vs Control | 94 |
| hsa-miR-656 | AAUAUUAUACAGUCAACCUCU | IPF DOWN vs Control | 95 |
| hsa-miR-362-3p | AACACACCUAUUCAAGGAUUCA | IPF DOWN vs Control | 96 |
| hsa-miR-340 | UUAUAAAGCAAUGAGACUGAUU | IPF DOWN vs Control | 97 |
| hsa-miR-451 | AAACCGUUACCAUUACUGAGUU | IPF DOWN vs Control | 98 |
| hsa-miR-423-5p | UGAGGGGCAGAGAGCGAGACUUU | IPF DOWN vs Control | 99 |
| hsa-miR-652 | AAUGGCGCCACUAGGGUUGUG | IPF DOWN vs Control | 100 |
| hsa-miR-127-3p | UCGGAUCCGUCUGAGCUUGGCU | IPF DOWN vs Control | 101 |
| hsa-miR-495 | AAACAAACAUGGUGCACUUCUU | IPF DOWN vs Control | 102 |
| hsa-miR-328 | CUGGCCCUCUCUGCCCUUCCGU | IPF DOWN vs Control | 103 |
| hsa-miR-590-5p | GAGCUUAUUCAUAAAAGUGCAG | IPF DOWN vs Control | 104 |
| hsa-miR-103 | AGCAGCAUUGUACAGGGCUAUGA | IPF DOWN vs Control | 105 |
| hsa-miR-19b | UGUGCAAAUCCAUGCAAAACUGA | IPF DOWN vs Control | 106 |
| hsa-miR-324-3p | ACUGCCCCAGGUGCUGCUGG | IPF DOWN vs Control | 107 |
| hsa-miR-145# | GGAUUCCUGGAAAUACUGUUCU | IPF DOWN vs Control | 108 |
| hsa-miR-199a-3p | ACAGUAGUCUGCACAUUGGUUA | IPF DOWN vs Control | 109 |
| hsa-miR-598 | UACGUCAUCGUUGUCAUCGUCA | IPF DOWN vs Control | 110 |
| hsa-miR-151-5P | UCGAGGAGCUCACAGUCUAGU | IPF DOWN vs Control | 111 |
| hsa-miR-130a | CAGUGCAAUGUUAAAAGGGCAU | IPF DOWN vs Control | 112 |
| hsa-miR-502-3p | AAUGCACCUGGGCAAGGAUUCA | IPF DOWN vs Control | 113 |
| hsa-miR-136# | CAUCAUCGUCUCAAAUGAGUCU | IPF DOWN vs Control | 114 |
| hsa-miR-194 | UGUAACAGCAACUCCAUGUGGA | IPF DOWN vs Control | 115 |
| hsa-miR-221 | AGCUACAUUGUCUGCUGGGUUUC | IPF DOWN vs Control | 116 |
| hsa-miR-22# | AGUUCUUCAGUGGCAAGCUUUA | IPF DOWN vs Control | 117 |
| hsa-miR-93 | CAAAGUGCUGUUCGUGCAGGUAG | IPF DOWN vs Control | 118 |
| hsa-miR-335 | UCAAGAGCAAUAACGAAAAAUGU | IPF DOWN vs Control | 119 |
| hsa-miR-24-2# | UGCCUACUGAGCUGAAACACAG | IPF DOWN vs Control | 120 |
| hsa-miR-130b | CAGUGCAAUGAUGAAAGGGCAU | IPF DOWN vs Control | 121 |
| hsa-miR-99b | CACCCGUAGAACCGACCUUGCG | IPF DOWN vs Control | 122 |
| hsa-miR-195 | UAGCAGCACAGAAAUAUUGGC | IPF DOWN vs Control | 123 |
| hsa-miR-411 | UAGUAGACCGUAUAGCGUACG | IPF DOWN vs Control | 124 |
| hsa-miR-29b | UAGCACCAUUUGAAAUCAGUGUU | IPF DOWN vs Control | 125 |
| hsa-miR-576-3p | AAGAUGUGGAAAAAUUGGAAUC | IPF DOWN vs Control | 126 |
| hsa-miR-340# | UCCGUCUCAGUUACUUUAUAGC | IPF DOWN vs Control | 127 |
| hsa-miR-148b# | AAGUUCUGUUAUACACUCAGGC | IPF DOWN vs Control | 128 |
| hsa-miR-212 | UAACAGUCUCCAGUCACGGCC | IPF DOWN vs Control | 129 |
| hsa-miR-152 | UCAGUGCAUGACAGAACUUGG | IPF DOWN vs Control | 130 |
| hsa-miR-143 | UGAGAUGAAGCACUGUAGCUC | IPF DOWN vs Control | 131 |
| dme-miR-7 | UGGAAGACUAGUGAUUUUGUUGU | IPF DOWN vs Control | 132 |
| hsa-miR-543 | AAACAUUCGCGGUGCACUUCUU | IPF DOWN vs Control | 133 |
| hsa-miR-30d# | CUUUCAGUCAGAUGUUUGCUGC | IPF DOWN vs Control | 134 |
| hsa-miR-213 | ACCAUCGACCGUUGAUUGUACC | IPF DOWN vs Control | 135 |
| hsa-miR-126# | CAUUAUUACUUUUGGUACGCG | IPF DOWN vs Control | 136 |
| hsa-miR-1197 | UAGGACACAUGGUCUACUUCU | IPF DOWN vs Control | 137 |
| hsa-miR-1255B | CGGAUGAGCAAAGAAAGUGGUU | IPF DOWN vs Control | 138 |
| hsa-miR-154# | AAUCAUACACGGUUGACCUAUU | IPF DOWN vs Control | 139 |
| hsa-miR-196b | UAGGUAGUUUCCUGUUGUUGGG | IPF DOWN vs Control | 140 |
| hsa-miR-21# | CAACACCAGUCGAUGGGCUGU | IPF DOWN vs Control | 141 |
| hsa-miR-335# | UUUUUCAUUAUUGCUCCUGACC | IPF DOWN vs Control | 142 |
| hsa-miR-33a# | CAAUGUUUCCACAGUGCAUCAC | IPF DOWN vs Control | 143 |
| hsa-miR-374a# | CUUAUCAGAUUGUAUUGUAAUU | IPF DOWN vs Control | 144 |
| hsa-miR-381 | UAUACAAGGGCAAGCUCUCUGU | IPF DOWN vs Control | 145 |
| hsa-miR-409-5p | AGGUUACCCGAGCAACUUUGCAU | IPF DOWN vs Control | 146 |
| hsa-miR-411# | UAUGUAACACGGUCCACUAACC | IPF DOWN vs Control | 147 |
| hsa-miR-548J | AAAAGUAAUUGCGGUCUUUGGU | IPF DOWN vs Control | 148 |
| hsa-miR-551b | GCGACCCAUACUUGGUUUCAG | IPF DOWN vs Control | 149 |
| hsa-miR-616 | AGUCAUUGGAGGGUUUGAGCAG | IPF DOWN vs Control | 150 |
| hsa-miR-638 | AGGGAUCGCGGGCGGGUGGCGGCCU | IPF DOWN vs Control | 151 |
| hsa-miR-664 | UAUUCAUUUAUCCCCAGCCUACA | IPF DOWN vs Control | 152 |
| hsa-miR-889 | UUAAUAUCGGACAACCAUUGU | IPF DOWN vs Control | 153 |
| mo-miR-29c# | UGACCGAUUUCUCCUGGUGUUC | IPF DOWN vs Control | 154 |

### Example 2

### IPF Patients were Determined to have a Unique miRNA Profile Compared to Healthy Controls

Materials and Methods:

Plasma samples were obtained from placebo-treated white male IPF patients. Plasma samples for demographically matched healthy control subjects with associated medical histories and medication use were obtained commercially. All plasma samples were collected in vials containing EDTA as the anticoagulant. All samples were obtained under appropriate written Informed Consent. Histories were reviewed for the following additional criteria: not a current smoker (both groups), lack of pulmonary or other significant disease (healthy control group), no recent use of prednisolone or other drugs used off label in the treatment of IPF (healthy control group), no use of prednisolone or other drugs for the treatment of IPF within 28 days prior to sample collection (IPF group). Summary demographics for IPF patients and healthy controls are shown in Table 4.

TABLE 4

| | Healthy | IPF |
|---|---|---|
| Total # | 13 | 15 |
| Ethnic origin White | 100% | 100% |
| Male | 100% | 100% |
| Age (year)^{a} | 60.8 | 64.7 |
| Weight (kg)^{b} | 87.1 | 89.4 |
| Smoking status | | |
| Never smoked | 46% | 33% |
| Previous smoker | 54% | 67% |
| Current smoker | 0 | 0 |
| FVC % predicted | — | 70.7 |
| DLco % predicted (Hgb-corrected) | — | 46.7 |
| 6 minute walk distance (m) | — | 440.1 |
| Death | — | 7% |
| ΔFVC % predicted (Baseline to Week 72) | — | −9.2 |
| Δ 6 minute walk distance (m) (Baseline to Week 72) | — | −46.9 |

Samples from IPF patients (n=15), healthy controls (n=15), and appropriate technical replicates (n=3 from IPF patients and n=3 from healthy controls) were grouped into two batches each for and RNA isolation by standard methods. Total RNA was extracted by standard methods. Isolated RNA was reverse transcribed and preamplified using the Applied Biosystems Megaplex RT and Preamplification Human Primer Pools according to manufacturer's protocols.

miRNAs were profiled by real-time PCR using the TaqMan Human miRNA Array set v3.0 (Cards A+B) according to manufacturer's protocols.

Two control samples were excluded due to poor data quality. For all remaining samples microRNAs with Ct values less than 35 were selected for data analysis. microRNAs for normalization were selected by a mean centering method (Wylie et al, BMC Research Notes, 4:555, 2011). Normalization and all subsequent analyses were performed in Partek Genomics Suite (Partek, St. Louis, Mo.). Differentially expressed miRNAs were identified by ANOVA with correction for multiple comparisons. Sequences detected in <50% of samples in both the IPF and control groups were excluded. miRNAs present in <50% of samples from one or the other group were evaluated as potentially disease-status specific sequences.

miRNAs identified as having increased presentation in IPF patient plasma relative to a predetermined criterion are set forth below in Table 5.

TABLE 5

| | | Differential | |
|---|---|---|---|
| microRNA ID | Sequence Data | Regulation | SEQ ID NO. |
| hsa-miR-130a | CAGUGCAAUGUUAAAAGGGCAU | IPF UP vs Control | 112 |
| hsa-miR-142-5p | CAUAAAGUAGAAAGCACUACU | IPF UP vs Control | 43 |
| hsa-miR-148a | UCAGUGCACUACAGAACUUUGU | IPF UP vs Control | 91 |
| hsa-miR-152 | UCAGUGCAUGACAGAACUUGG | IPF UP vs Control | 130 |
| hsa-miR-15a# | CAGGCCAUAUUGUGCUGCCUCA | IPF UP vs Control | 64 |
| hsa-miR-19a | UGUGCAAAUCUAUGCAAAACUGA | IPF UP vs Control | 74 |
| hsa-miR-21 | UAGCUUAUCAGACUGAUGUUGA | IPF UP vs Control | 51 |
| hsa-miR-21# | CAACACCAGUCGAUGGGCUGU | IPF UP vs Control | 141 |
| hsa-miR-26a-2# | CCUAUUCUUGAUUACUUGUUUC | IPF UP vs Control | 82 |
| hsa-miR-324-3p | ACUGCCCCAGGUGCUGCUGG | IPF UP vs Control | 107 |
| hsa-miR-335 | UCAAGAGCAAUAACGAAAAAUGU | IPF UP vs Control | 119 |
| hsa-miR-362-3p | AACACACCUAUUCAAGGAUUCA | IPF UP vs Control | 96 |
| hsa-miR-590-5p | GAGCUUAUUCAUAAAAGUGCAG | IPF UP vs Control | 104 |
| hsa-miR-598 | UACGUCAUCGUUGUCAUCGUCA | IPF UP vs Control | 110 |
| hsa-miR-122 | UGGAGUGUGACAAUGGUGUUUG | IPF UP vs Control | 22 |
| hsa-miR-146a | UGAGAACUGAAUUCCAUGGGUU | IPF UP vs Control | 21 |
| hsa-miR-146b-5p | UGAGAACUGAAUUCCAUAGGCU | IPF UP vs Control | 24 |
| hsa-miR-150 | UCUCCCAACCCUUGUACCAGUG | IPF UP vs Control | 27 |
| hsa-miR-222 | AGCUACAUCUGGCUACUGGGU | IPF UP vs Control | 16 |
| hsa-miR-345 | GCUGACUCCUAGUCCAGGGCUC | IPF UP vs Control | 18 |
| hsa-let-7a# | CUAUACAAUCUACUGUCUUUC | IPF UP vs Control | 155 |
| hsa-miR-106a | AAAAGUGCUUACAGUGCAGGUAG | IPF UP vs Control | 156 |
| hsa-miR-1227 | CGUGCCACCCUUUUCCCCAG | IPF UP vs Control | 157 |
| hsa-miR-128 | UCACAGUGAACCGGUCUCUUU | IPF UP vs Control | 158 |
| hsa-miR-132 | UAACAGUCUACAGCCAUGGUCG | IPF UP vs Control | 159 |
| hsa-miR-140-5p | CAGUGGUUUUACCCUAUGGUAG | IPF UP vs Control | 160 |
| hsa-miR-141 | UAACACUGUCUGGUAAAGAUGG | IPF UP vs Control | 161 |
| hsa-miR-17 | CAAAGUGCUUACAGUGCAGGUAG | IPF UP vs Control | 162 |
| hsa-miR-185 | UGGAGAGAAAGGCAGUUCCUGA | IPF UP vs Control | 163 |
| hsa-miR-30a-5p | UGUAAACAUCCUCGACUGGAAG | IPF UP vs Control | 164 |
| hsa-miR-30d | UGUAAACAUCCCCGACUGGAAG | IPF UP vs Control | 165 |
| hsa-miR-34a | UGGCAGUGUCUUAGCUGGUUGU | IPF UP vs Control | 166 |
| hsa-miR-378 | ACUGGACUUGGAGUCAGAAGG | IPF UP vs Control | 167 |
| hsa-miR-425 | AAUGACACGAUCACUCCCGUUGA | IPF UP vs Control | 168 |
| hsa-miR-429 | UAAUACUGUCUGGUAAAACCGU | IPF UP vs Control | 169 |
| hsa-miR-579 | UUCAUUUGGUAUAAACCGCGAUU | IPF UP vs Control | 170 |
| hsa-miR-523 | GAACGCGCUUCCCUAUAGAGGGU | IPF UP vs Control | 171 |
| hsa-miR-551b# | GAAAUCAAGCGUGGGUGAGACC | IPF UP vs Control | 172 |

miRNAs identified as having decreased presentation in IPF patient plasma relative to a predetermined criterion are set forth below in Table 6.

TABLE 6

| | | Differential | |
|---|---|---|---|
| microRNA ID | Sequence Data | Regulation | SEQ ID NO. |
| hsa-let-7d | AGAGGUAGUAGGUUGCAUAGUU | IPF DOWN vs Control | 45 |
| hsa-let-7e | UGAGGUAGGAGGUUGUAUAGUU | IPF DOWN vs Control | 93 |
| hsa-let-7g | UGAGGUAGUAGUUUGUACAGUU | IPF DOWN vs Control | 72 |
| hsa-miR-103 | AGCAGCAUUGUACAGGGCUAUGA | IPF DOWN vs Control | 105 |
| hsa-miR-127-3p | UCGGAUCCGUCUGAGCUUGGCU | IPF DOWN vs Control | 101 |
| hsa-miR-142-3p | UGUAGUGUUUCCUACUUUAUGGA | IPF DOWN vs Control | 38 |
| hsa-miR-144# | GGAUAUCAUCAUAUACUGUAAG | IPF DOWN vs Control | 69 |
| hsa-miR-148b# | AAGUUCUGUUAUACACUCAGGC | IPF DOWN vs Control | 128 |
| hsa-miR-154# | AAUCAUACACGGUUGACCUAUU | IPF DOWN vs Control | 139 |
| hsa-miR-15b | UAGCAGCACAUCAUGGUUUACA | IPF DOWN vs Control | 56 |
| hsa-miR-190 | UGAUAUGUUUGAUAUAUUAGGU | IPF DOWN vs Control | 63 |
| hsa-miR-196b | UAGGUAGUUUCCUGUUGUUGGG | IPF DOWN vs Control | 140 |
| hsa-miR-20a# | ACUGCAUUAUGAGCACUUAAAG | IPF DOWN vs Control | 75 |
| hsa-miR-24-2# | UGCCUACUGAGCUGAAACACAG | IPF DOWN vs Control | 120 |
| hsa-miR-26a | UUCAAGUAAUCCAGGAUAGGCU | IPF DOWN vs Control | 70 |
| hsa-miR-28-5p | AAGGAGCUCACAGUCUAUUGAG | IPF DOWN vs Control | 90 |
| hsa-miR-29b | UAGCACCAUUUGAAAUCAGUGUU | IPF DOWN vs Control | 125 |
| hsa-miR-301a | CAGUGCAAUAGUAUUGUCAAAGC | IPF DOWN vs Control | 67 |
| hsa-miR-30b | UGUAAACAUCCUACACUCAGCU | IPF DOWN vs Control | 42 |
| hsa-miR-30c | UGUAAACAUCCUACACUCUCAGC | IPF DOWN vs Control | 52 |
| hsa-miR-340# | UCCGUCUCAGUUACUUUAUAGC | IPF DOWN vs Control | 127 |
| hsa-miR-374a | UUAUAAUACAACCUGAUAAGUG | IPF DOWN vs Control | 68 |
| hsa-miR-411 | UAGUAGACCGUAUAGCGUACG | IPF DOWN vs Control | 124 |
| hsa-miR-411# | UAUGUAACACGGUCCACUAACC | IPF DOWN vs Control | 147 |
| hsa-miR-543 | AAACAUUCGCGGUGCACUUCUU | IPF DOWN vs Control | 133 |
| hsa-miR-548J | AAAAGUAAUUGCGGUCUUUGGU | IPF DOWN vs Control | 148 |
| hsa-miR-664 | UAUUCAUUUAUCCCCAGCCUACA | IPF DOWN vs Control | 152 |
| hsa-miR-99b | CACCCGUAGAACCGACCUUGCG | IPF DOWN vs Control | 122 |
| hsa-miR-1244 | AAGUAGUUGGUUUGUAUGAGAUGGUU | IPF DOWN vs Control | 20 |
| hsa-let-7a | UGAGGUAGUAGGUUGUAUAGUU | IPF DOWN vs Control | 173 |
| hsa-let-7f | UGAGGUAGUAGAUUGUAUAGUU | IPF DOWN vs Control | 174 |
| hsa-miR-107 | AGCAGCAUUGUACAGGGCUAUCA | IPF DOWN vs Control | 175 |
| hsa-miR-125a-5p | UCCCUGAGACCCUUUAACCUGUGA | IPF DOWN vs Control | 176 |
| hsa-miR-181a-2# | ACCACUGACCGUUGACUGUACC | IPF DOWN vs Control | 177 |
| hsa-miR-19b-1# | AGUUUUGCAGGUUUGCAUCCAGC | IPF DOWN vs Control | 178 |
| hsa-miR-200c | UAAUACUGCCGGGUAAUGAUGGA | IPF DOWN vs Control | 179 |
| hsa-miR-27b# | AGAGCUUAGCUGAUUGGUGAAC | IPF DOWN vs Control | 180 |
| hsa-miR-331-3p | GCCCCUGGGCCUAUCCUAGAA | IPF DOWN vs Control | 181 |
| hsa-miR-339-5p | UCCCUGUCCUCCAGGAGCUCACG | IPF DOWN vs Control | 182 |
| hsa-miR-362-5p | AAUCCUUGGAACCUAGGUGUGAGU | IPF DOWN vs Control | 183 |
| hsa-miR-370 | GCCUGCUGGGGUGGAACCUGGU | IPF DOWN vs Control | 184 |
| hsa-miR-374b | AUAUAAUACAACCUGCUAAGUG | IPF DOWN vs Control | 185 |
| hsa-miR-379 | UGGUAGACUAUGGAACGUAGG | IPF DOWN vs Control | 186 |
| hsa-miR-454 | UAGUGCAAUAUUGCUUAUAGGGU | IPF DOWN vs Control | 187 |
| hsa-miR-520a-3p | AAAGUGCUUCCCUUUGGACUGU | IPF DOWN vs Control | 188 |
| hsa-miR-539 | GGAGAAAUUAUCCUUGGUGUGU | IPF DOWN vs Control | 189 |
| hsa-miR-758 | UUUGUGACCUGGUCCACUAACC | IPF DOWN vs Control | 190 |
| hsa-miR-766 | ACUCCAGCCCCACAGCCUCAGC | IPF DOWN vs Control | 191 |
| hsa-miR-9 | UCUUUGGUUAUCUAGCUGUAUGA | IPF DOWN vs Control | 192 |
| hsa-miR-98 | UGAGGUAGUAAGUUGUAUUGUU | IPF DOWN vs Control | 193 |
| hsa-miR-668 | UGUCACUCGGCUCGGCCCACUAC | IPF DOWN vs Control | 194 |
| hsa-miR-1256 | AGGCAUUGACUUCUCACUAGCU | IPF DOWN vs Control | 195 |
| hsa-miR-299-5p | UGGUUUACCGUCCCACAUACAU | IPF DOWN vs Control | 196 |

The data provided in Tables 5 and 6 above, shows that unique miRNA profiles are present in IPF patients compared to healthy controls subjects and that these unique profiles are detectable in a blood sample of the patient. The data presented herein demonstrate that the levels of one or more miRNA detected in the blood sample of a human subject are useful tools for the diagnosis of IPF.

### Example 3

### IPF Patients were Determined to have a Unique miRNA Profile Compared to Healthy Controls in a Further Study

Materials and Methods:

Plasma samples were obtained from placebo-treated white male IPF patients. Plasma samples for demographically matched healthy control subjects with associated medical histories and medication use were obtained commercially. All samples were collected in vials containing EDTA as the anticoagulant. All samples were obtained under appropriate written Informed Consent. Histories were reviewed for the following additional criteria: not a current smoker (both groups), lack of pulmonary or other significant disease (healthy control group), no recent use of prednisolone or other drugs used off label in the treatment of IPF (healthy control group), no use of prednisolone or other drugs for the treatment of IPF within 28 days prior to sample collection (IPF group). Summary demographics for IPF patients and healthy controls are shown in Table 7.

TABLE 7

| | Healthy controls | Progressive | Stable |
|---|---|---|---|
| | (n = 15) | (n = 15) | (n = 15) |
| Ethnic origin white | 100% | 100% | 100% |
| Male | 100% | 100% | 100% |
| Age (year) | 64.8 | 66.4 | 66.3 |
| BMI | 27.7 | 28.7 | 28.6 |
| Weight (kg) | 84.5 | 87.5 | 87.2 |
| Current/previous/never smoker | 13/27/60% | 0/60/40% | 0/67/33% |
| Baseline | | | |
| FVC % predicted | | 70.6 | 70.1 |
| DL_{co} % predicted (Hgb-corrected) | | 45.2 | 48.0 |
| 6-minute walk distance (m) | | 453.2 | 417.9 |
| Outcomes | | | |
| Death | | 7% | 0% |
| ΔFVC % predicted | | −11.4% | −2.6% |
| (Baseline to Week 72) | | | |
| Δ6MW (Baseline to Week 72; m) | | −52.1 | 23.07 |
| ΔFVC % predicted | | −10.6 | 0.6 |
| (24 week change) | | | |
| ΔFVC % predicted | | −12.4 | −1.0 |
| (48 week change) | | | |

Samples from IPF patients (n=30, including 15 progressive IPF and 15 stable IPF)), healthy controls (n=15) were grouped into two batches each for and RNA isolation by standard methods. Total RNA was extracted by standard methods. Isolated RNA was reverse transcribed and preamplified using the Applied Biosystems Megaplex RT and Preamplification Human Primer Pools according to manufacturer's protocols.

miRNAs were profiled by real-time PCR using the TaqMan Human miRNA Array set v3.0 (Cards A+B) according to manufacturer's protocols.

microRNAs with Ct values less than 35 were selected for data analysis. microRNAs for normalization were selected by a mean centering method (Wylie et al, BMC Research Notes, 4:555, 2011). Normalization and all subsequent analyses were performed in Partek Genomics Suite (Partek, St. Louis, Mo.). Differentially expressed miRNAs were identified by ANOVA with correction for multiple comparisons. Sequences detected in <50% of samples in both the IPF and control groups were excluded. miRNAs present in <50% of samples from one or the other group were evaluated as potentially disease-status specific sequences. Differentially expressed miRNAs between progressive IPF and stable IPF patients were identified in a similar manner.

miRNAs identified as having increased presentation in IPF patient plasma relative to a predetermined criterion are set forth below in Table 8.

TABLE 8

| | | Differential | SEQ ID |
|---|---|---|---|
| microRNA ID | Sequence Data | Regulation | NO. |
| hsa-let-7b | UGAGGUAGUAGGUUGUGUGGUU | IPF UP vs Control | 76 |
| hsa-miR-106a | AAAAGUGCUUACAGUGCAGGUAG | IPF UP vs Control | 156 |
| hsa-miR-10b# | ACAGAUUCGAUUCUAGGGGAAU | IPF UP vs Control | 5 |
| hsa-miR-1183 | CACUGUAGGUGAUGGUGAGAGUGGGCA | IPF UP vs control | 197 |
| hsa-miR-122 | UGGAGUGUGACAAUGGUGUUUG | IPF UP vs Control | 22 |
| hsa-miR-1227 | CGUGCCACCCUUUUCCCCAG | IPF UP vs Control | 157 |
| hsa-miR-1233 | UGAGCCCUGUCCUCCCGCAG | IPF UP vs control | 198 |
| hsa-miR-1247 | ACCCGUCCCGUUCGUCCCCGGA | IPF UP vs control | 199 |
| hsa-miR-1270 | CUGGAGAUAUGGAAGAGCUGUGU | IPF UP vs control | 201 |
| hsa-miR-1274A | GUCCCUGUUCAGGCGCCA | IPF UP vs control | 202 |
| hsa-miR-1275 | GUGGGGGAGAGGCUGUC | IPF UP vs control | 203 |
| hsa-miR-1290 | UGGAUUUUUGGAUCAGGGA | IPF UP vs Control | 34 |
| hsa-miR-1298 | UUCAUUCGGCUGUCCAGAUGUA | IPF UP vs control | 204 |
| hsa-miR-1303 | UUUAGAGACGGGGUCUUGCUCU | IPF UP vs Control | 3 |
| hsa-miR-132 | UAACAGUCUACAGCCAUGGUCG | IPF UP vs Control | 159 |
| hsa-miR-135b | UAUGGCUUUUCAUUCCUAUGUGA | IPF UP vs control | 205 |
| hsa-miR-138 | AGCUGGUGUUGUGAAUCAGGCCG | IPF UP vs control | 206 |
| hsa-miR-146a | UGAGAACUGAAUUCCAUGGGUU | IPF UP vs Control | 21 |
| hsa-miR-17 | CAAAGUGCUUACAGUGCAGGUAG | IPF UP vs Control | 162 |
| hsa-miR-186 | CAAAGAAUUCUCCUUUUGGGCU | IPF UP vs control | 210 |
| hsa-miR-193a-3p | AACUGGCCUACAAAGUCCCAGU | IPF UP vs control | 212 |
| hsa-miR-193b | AACUGGCCCUCAAAGUCCCGCU | IPF UP vs Control | 12 |
| hsa-miR-197 | UUCACCACCUUCUCCACCCAGC | IPF UP vs Control | 10 |
| hsa-miR-200a | UAACACUGUCUGGUAACGAUGU | IPF UP vs control | 214 |
| hsa-miR-205 | UCCUUCAUUCCACCGGAGUCUG | IPF UP vs control | 215 |
| hsa-miR-206 | UGGAAUGUAAGGAAGUGUGUGG | IPF UP vs Control | 23 |
| hsa-miR-20b | CAAAGUGCUCAUAGUGCAGGUAG | IPF UP vs control | 216 |
| hsa-miR-214 | ACAGCAGGCACAGACAGGCAGU | IPF UP vs control | 217 |
| hsa-miR-214# | UGCCUGUCUACACUUGCUGUGC | IPF UP vs control | 218 |
| hsa-miR-218 | UUGUGCUUGAUCUAACCAUGU | IPF UP vs control | 219 |
| hsa-miR-220b | CCACCACCGUGUCUGACACUU | IPF UP vs control | 220 |
| hsa-miR-222 | AGCUACAUCUGGCUACUGGGU | IPF UP vs Control | 16 |
| hsa-miR-223 | UGUCAGUUUGUCAAAUACCCCA | IPF UP vs control | 221 |
| hsa-miR-26a-2# | CCUAUUCUUGAUUACUUGUUUC | IPF UP vs Control | 82 |
| hsa-miR-30a-3p | CUUUCAGUCGGAUGUUUGCAGC | IPF UP vs control | 223 |
| hsa-miR-320 | AAAAGCUGGGUUGAGAGGGCGA | IPF UP vs Control | 37 |
| hsa-miR-326 | CCUCUGGGCCCUUCCUCCAG | IPF UP vs control | 225 |
| hsa-miR-338-5P | AACAAUAUCCUGGUGCUGAGUG | IPF UP vs Control | 15 |
| hsa-miR-345 | GCUGACUCCUAGUCCAGGGCUC | IPF UP vs Control | 18 |
| hsa-miR-346 | UGUCUGCCCGCAUGCCUGCCUCU | IPF UP vs control | 226 |
| hsa-miR-34a | UGGCAGUGUCUUAGCUGGUUGU | IPF UP vs Control | 166 |
| hsa-miR-34a# | CAAUCAGCAAGUAUACUGCCCU | IPF UP vs Control | 13 |
| hsa-miR-375 | UUUGUUCGUUCGGCUCGCGUGA | IPF UP vs Control | 8 |
| hsa-miR-429 | UAAUACUGUCUGGUAAAACCGU | IPF UP vs Control | 169 |
| hsa-miR-450a | UUUUGCGAUGUGUUCCUAAUAU | IPF UP vs control | 228 |
| hsa-miR-450b-5p | UUUUGCAAUAUGUUCCUGAAUA | IPF UP vs control | 229 |
| hsa-miR-455-5p | UAUGUGCCUUUGGACUACAUCG | IPF UP vs control | 230 |
| hsa-miR-501-5p | AAUCCUUUGUCCCUGGGUGAGA | IPF UP vs control | 234 |
| hsa-miR-511 | GUGUCUUUUGCUCUGCAGUCA | IPF UP vs control | 236 |
| hsa-miR-518d-3p | CAAAGCGCUUCCCUUUGGAGC | IPF UP vs control | 237 |
| hsa-miR-518e | AAAGCGCUUCCCUUCAGAGUG | IPF UP vs control | 238 |
| hsa-miR-518f | GAAAGCGCUUCUCUUUAGAGG | IPF UP vs control | 239 |
| hsa-miR-548a-3p | CAAAACUGGCAAUUACUUUUGC | IPF UP vs Control | 14 |
| hsa-miR-548c-3p | CAAAAAUCUCAAUUACUUUUGC | IPF UP vs control | 240 |
| hsa-miR-570 | CGAAAACAGCAAUUACCUUUGC | IPF UP vs control | 241 |
| hsa-miR-571 | UGAGUUGGCCAUCUGAGUGAG | IPF UP vs control | 242 |
| hsa-miR-574-3p | CACGCUCAUGCACACACCCACA | IPF UP vs Control | 4 |
| hsa-miR-577 | UAGAUAAAAUAUUGGUACCUG | IPF UP vs control | 243 |
| hsa-miR-590-5p | GAGCUUAUUCAUAAAAGUGCAG | IPF UP vs Control | 104 |
| hsa-miR-598 | UACGUCAUCGUUGUCAUCGUCA | IPF UP vs Control | 110 |
| hsa-miR-618 | AAACUCUACUUGUCCUUCUGAGU | IPF UP vs control | 244 |
| hsa-miR-885-5p | UCCAUUACACUACCCUGCCUCU | IPF UP vs control | 247 |
| hsa-miR-9# | AUAAAGCUAGAUAACCGAAAGU | IPF UP vs control | 249 |
| hsa-miR-95 | UUCAACGGGUAUUUAUUGAGCA | IPF UP vs control | 250 |

miRNAs identified as having decreased presentation in IPF patient plasma relative to a control are set forth below in Table 9.

TABLE 9

| | | Differential | SEQ ID |
|---|---|---|---|
| microRNA ID | Sequence Data | Regulation | NO. |
| hsa-let-7a | UGAGGUAGUAGGUUGUAUAGUU | IPF DOWN vs Control | 173 |
| hsa-let-7d | AGAGGUAGUAGGUUGCAUAGUU | IPF DOWN vs Control | 45 |
| hsa-let-7e | UGAGGUAGGAGGUUGUAUAGUU | IPF DOWN vs Control | 93 |
| hsa-let-7f | UGAGGUAGUAGAUUGUAUAGUU | IPF DOWN vs Control | 174 |
| hsa-let-7g | UGAGGUAGUAGUUUGUACAGUU | IPF DOWN vs Control | 72 |
| hsa-miR-103 | AGCAGCAUUGUACAGGGCUAUGA | IPF DOWN vs Control | 105 |
| hsa-miR-106b# | CCGCACUGUGGGUACUUGCUGC | IPF DOWN vs Control | 92 |
| hsa-miR-107 | AGCAGCAUUGUACAGGGCUAUCA | IPF DOWN vs Control | 175 |
| hsa-miR-1244 | AAGUAGUUGGUUUGUAUGAGAUGGUU | IPF DOWN vs Control | 20 |
| hsa-miR-1249 | ACGCCCUUCCCCCCCUUCUUCA | IPF DOWN vs control | 200 |
| hsa-miR-125a-5p | UCCCUGAGACCCUUUAACCUGUGA | IPF DOWN vs Control | 176 |
| hsa-miR-1260 | AUCCCACCUCUGCCACCA | IPF DOWN vs Control | 47 |
| hsa-miR-127-3p | UCGGAUCCGUCUGAGCUUGGCU | IPF DOWN vs Control | 101 |
| hsa-miR-142-3p | UGUAGUGUUUCCUACUUUAUGGA | IPF DOWN vs Control | 38 |
| hsa-miR-144# | GGAUAUCAUCAUAUACUGUAAG | IPF DOWN vs Control | 69 |
| hsa-miR-145 | GUCCAGUUUUCCCAGGAAUCCCU | IPF DOWN vs control | 207 |
| hsa-miR-148b# | AAGUUCUGUUAUACACUCAGGC | IPF DOWN vs Control | 128 |
| hsa-miR-151-5P | UCGAGGAGCUCACAGUCUAGU | IPF DOWN vs Control | 111 |
| hsa-miR-15a | UAGCAGCACAUAAUGGUUUGUG | IPF DOWN vs control | 208 |
| hsa-miR-15b | UAGCAGCACAUCAUGGUUUACA | IPF DOWN vs Control | 56 |
| hsa-miR-181a | AACAUUCAACGCUGUCGGUGAGU | IPF DOWN vs Control | 66 |
| hsa-miR-181a-2# | ACCACUGACCGUUGACUGUACC | IPF DOWN vs Control | 177 |
| hsa-miR-181c | AACAUUCAACCUGUCGGUGAGU | IPF DOWN vs control | 209 |
| hsa-miR-18a# | ACUGCCCUAAGUGCUCCUUCUGG | IPF DOWN vs control | 211 |
| hsa-miR-190 | UGAUAUGUUUGAUAUAUUAGGU | IPF DOWN vs Control | 63 |
| hsa-miR-194 | UGUAACAGCAACUCCAUGUGGA | IPF DOWN vs Control | 115 |
| hsa-miR-196b | UAGGUAGUUUCCUGUUGUUGGG | IPF DOWN vs Control | 140 |
| hsa-miR-199a-5p | CCCAGUGUUCAGACUACCUGUUC | IPF DOWN vs Control | 81 |
| hsa-miR-199b-5p | CCCAGUGUUUAGACUAUCUGUUC | IPF DOWN vs control | 213 |
| hsa-miR-19b-1# | AGUUUUGCAGGUUUGCAUCCAGC | IPF DOWN vs Control | 178 |
| hsa-miR-200c | UAAUACUGCCGGGUAAUGAUGGA | IPF DOWN vs Control | 179 |
| hsa-miR-20a | UAAAGUGCUUAUAGUGCAGGUAG | IPF DOWN vs Control | 89 |
| hsa-miR-20a# | ACUGCAUUAUGAGCACUUAAAG | IPF DOWN vs Control | 75 |
| hsa-miR-23b | AUCACAUUGCCAGGGAUUACC | IPF DOWN vs control | 222 |
| hsa-miR-24-2# | UGCCUACUGAGCUGAAACACAG | IPF DOWN vs Control | 120 |
| hsa-miR-26a | UUCAAGUAAUCCAGGAUAGGCU | IPF DOWN vs Control | 70 |
| hsa-miR-27b# | AGAGCUUAGCUGAUUGGUGAAC | IPF DOWN vs Control | 180 |
| hsa-miR-28-5p | AAGGAGCUCACAGUCUAUUGAG | IPF DOWN vs Control | 90 |
| hsa-miR-29b | UAGCACCAUUUGAAAUCAGUGUU | IPF DOWN vs Control | 125 |
| hsa-miR-301a | CAGUGCAAUAGUAUUGUCAAAGC | IPF DOWN vs Control | 67 |
| hsa-miR-30b | UGUAAACAUCCUACACUCAGCU | IPF DOWN vs Control | 42 |
| hsa-miR-30c | UGUAAACAUCCUACACUCUCAGC | IPF DOWN vs Control | 52 |
| hsa-miR-30e-3p | CUUUCAGUCGGAUGUUUACAGC | IPF DOWN vs control | 224 |
| hsa-miR-324-5p | CGCAUCCCCUAGGGCAUUGGUGU | IPF DOWN vs Control | 73 |
| hsa-miR-331-3p | GCCCCUGGGCCUAUCCUAGAA | IPF DOWN vs Control | 181 |
| hsa-miR-339-5p | UCCCUGUCCUCCAGGAGCUCACG | IPF DOWN vs Control | 182 |
| hsa-miR-340# | UCCGUCUCAGUUACUUUAUAGC | IPF DOWN vs Control | 127 |
| hsa-miR-362-5p | AAUCCUUGGAACCUAGGUGUGAGU | IPF DOWN vs Control | 183 |
| hsa-miR-370 | GCCUGCUGGGGUGGAACCUGGU | IPF DOWN vs Control | 184 |
| hsa-miR-374a | UUAUAAUACAACCUGAUAAGUG | IPF DOWN vs Control | 68 |
| hsa-miR-374b | AUAUAAUACAACCUGCUAAGUG | IPF DOWN vs Control | 185 |
| hsa-miR-379 | UGGUAGACUAUGGAACGUAGG | IPF DOWN vs Control | 186 |
| hsa-miR-411 | UAGUAGACCGUAUAGCGUACG | IPF DOWN vs Control | 124 |
| hsa-miR-431 | UGUCUUGCAGGCCGUCAUGCA | IPF DOWN vs control | 227 |
| hsa-miR-454 | UAGUGCAAUAUUGCUUAUAGGGU | IPF DOWN vs Control | 187 |
| hsa-miR-487a | AAUCAUACAGGGACAUCCAGUU | IPF DOWN vs control | 231 |
| hsa-miR-493 | UGAAGGUCUACUGUGUGCCAGG | IPF DOWN vs control | 232 |
| hsa-miR-494 | UGAAACAUACACGGGAAACCUC | IPF DOWN vs control | 233 |
| hsa-miR-495 | AAACAAACAUGGUGCACUUCUU | IPF DOWN vs Control | 102 |
| hsa-miR-505# | GGGAGCCAGGAAGUAUUGAUGU | IPF DOWN vs control | 235 |
| hsa-miR-539 | GGAGAAAUUAUCCUUGGUGUGU | IPF DOWN vs Control | 189 |
| hsa-miR-543 | AAACAUUCGCGGUGCACUUCUU | IPF DOWN vs Control | 133 |
| hsa-miR-548J | AAAAGUAAUUGCGGUCUUUGGU | IPF DOWN vs Control | 148 |
| hsa-miR-664 | UAUUCAUUUAUCCCCAGCCUACA | IPF DOWN vs Control | 152 |
| hsa-miR-744# | CUGUUGCCACUAACCUCAACCU | IPF DOWN vs control | 245 |
| hsa-miR-758 | UUUGUGACCUGGUCCACUAACC | IPF DOWN vs Control | 190 |
| hsa-miR-766 | ACUCCAGCCCCACAGCCUCAGC | IPF DOWN vs Control | 191 |
| hsa-miR-769-5p | UGAGACCUCUGGGUUCUGAGCU | IPF DOWN vs control | 246 |
| hsa-miR-9 | UCUUUGGUUAUCUAGCUGUAUGA | IPF DOWN vs Control | 192 |
| hsa-miR-98 | UGAGGUAGUAAGUUGUAUUGUU | IPF DOWN vs Control | 193 |
| hsa-miR-99b | CACCCGUAGAACCGACCUUGCG | IPF DOWN vs Control | 122 |

The data provided in Tables 8 and 9 above, shows that unique miRNA profiles are present in IPF patients compared to healthy controls subjects and that these unique profiles are detectable in a blood sample of the patient. In addition, analysis of the miRNAs in the samples indicated that two miRNAs (i.e., miR-1183 (SEQ ID NO:197) and miR-892b (SEQ ID NO:248)) have decreased presentation in progressive IPF compared to stable IPF patients. The data presented herein demonstrate that the levels of one or more miRNA detected in the blood sample of a human subject are useful tools for the diagnosis of IPF.

All of the references cited herein, including patents, patent applications, literature publications, and the like, are hereby incorporated in their entireties by reference.

While this invention has been described with an emphasis upon preferred embodiments, it will be obvious to those of ordinary skill in the art that variations of the preferred compounds and methods may be used and that it is intended that the invention may be practiced otherwise than as specifically described herein. Accordingly, this invention includes all modifications encompassed within the spirit and scope of the invention as defined by the following claims.

1. A method of diagnosing idiopathic pulmonary fibrosis (IPF) in a human subject comprising detecting in a blood sample of from the subject the level of one, two, three, four, five six, seven or more microRNAs, wherein an increase or decrease in the level of the one or more microRNA relative to a predetermined criterion is indicative of a diagnosis of IPF.

2. The method of claim 1 further comprising the step of comparing the level of the microRNA to a predetermined criterion or range.

3. The method of claim 1, wherein the one, two, three, four, five, six, seven or more microRNA are selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:11l), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a# (SEQ ID NO: 155), miR-106a (SEQ ID NO:156), miR-1227 (SEQ ID NO:157), miR-128 (SEQ ID NO: 158), miR-132 (SEQ ID NO:159), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO: 179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO: 191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), miR-1183 (SEQ ID NO:197), miR-1233 (SEQ ID NO:198), miR-1247 (SEQ ID NO:199), miR-1249 (SEQ ID NO:200), miR-1270 (SEQ ID NO:201), miR-1274A (SEQ ID NO:202), miR-1275 (SEQ ID NO: 203), miR-1298 (SEQ ID NO:204), miR-135b (SEQ ID NO:205), miR-138 (SEQ ID NO: 206), miR-145 (SEQ ID NO:207), miR-15a (SEQ ID NO:208), miR-181c (SEQ ID NO: 209), miR-186 (SEQ ID NO:210), miR-18a# (SEQ ID NO:211), miR-193a-3p (SEQ ID NO:212), miR-199b-5p (SEQ ID NO:213), miR-200a (SEQ ID NO:214), miR-205 (SEQ ID NO:215), miR-20b (SEQ ID NO:216), miR-214 (SEQ ID NO:217), miR-214# (SEQ ID NO:218), miR-218 (SEQ ID NO:219), miR-220b (SEQ ID NO:220), miR-223 (SEQ ID NO:221), miR-23b (SEQ ID NO:222), miR-30a-3p (SEQ ID NO:223), miR-30e-3p (SEQ ID NO:224), miR-326 (SEQ ID NO:225), miR-346 (SEQ ID NO:226), miR-431 (SEQ ID NO:227), miR-450a (SEQ ID NO:228), miR-450b-5p (SEQ ID NO:229), miR-455-5p (SEQ ID NO:230), miR-487a (SEQ ID NO:231), miR-493 (SEQ ID NO:232), miR-494 (SEQ ID NO:233), miR-501-5p (SEQ ID NO:234), miR-505# (SEQ ID NO:235), miR-511 (SEQ ID NO:236), miR-518d-3p (SEQ ID NO:237), miR-518e (SEQ ID NO:238), miR-518f (SEQ ID NO:239), miR-548c-3p (SEQ ID NO:240), miR-570 (SEQ ID NO:241), miR-571 (SEQ ID NO:242), miR-577 (SEQ ID NO:243), miR-618 (SEQ ID NO:244), miR-744# (SEQ ID NO:245), miR-769-5p (SEQ ID NO:246), miR-885-5p (SEQ ID NO: 247), miR-892b (SEQ ID NO:248), miR-9# (SEQ ID NO:249), miR-95 (SEQ ID NO:250), and combinations thereof.

4-17. (canceled)

18. The method of claim 1, wherein the one, two, three, four, five, six, seven or more microRNA are selected from the group consisting of
(a) let-7a (SEQ ID NO:173), let-7d (SEQ ID NO:45), let-7e (SEQ ID NO:93), let-7f (SEQ ID NO:174), let-7g (SEQ ID NO:72); miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-379 (SEQ ID NO:186), miR-411 (SEQ ID NO:124), miR-454 (SEQ ID NO:187), miR-539 (SEQ ID NO:189), miR-543 (SEQ ID NO:133), miR-548J (SEQ ID NO:148), miR-664 (SEQ ID NO:152), miR-758 (SEQ ID NO:190), miR-766 (SEQ ID NO:191), miR-9 (SEQ ID NO:192), miR-98 (SEQ ID NO:193), miR-99b (SEQ ID NO:122); miR-668 (SEQ ID NO:194), miR-106a (SEQ ID NO:156), miR-122 (SEQ ID NO:22), miR-1227 (SEQ ID NO:157), miR-132 (SEQ ID NO: 159), miR-146a (SEQ ID NO:21), miR-17 (SEQ ID NO:162), miR-222 (SEQ ID NO:16), miR-26a-2# (SEQ ID NO:82), miR-345 (SEQ ID NO:18), miR-34a (SEQ ID NO:166), miR-429 (SEQ ID NO:169), miR-590-5p (SEQ ID NO:104), miR-598 (SEQ ID NO:110), miR-551b# (SEQ ID NO:172), miR-1183 (SEQ ID NO:197) and miR-892b, (SEQ ID NO: 248) and combinations thereof;
(b) let-7a (SEQ ID NO:173), let-7d (SEQ ID NO:45), let-7e (SEQ ID NO:93), let-7f (SEQ ID NO:174), miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-20a# (SEQ ID NO:75), miR-24-2# (SEQ ID NO:120), miR-26a (SEQ ID NO:70), miR-28-5p (SEQ ID NO:90), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-379 (SEQ ID NO:186), miR-411 (SEQ ID NO:124), miR-539 (SEQ ID NO:189), miR-543 (SEQ ID NO:133), miR-664 (SEQ ID NO:152), miR-758 (SEQ ID NO:190), miR-9 (SEQ ID NO:192), miR-98 (SEQ ID NO:193). miR-99b (SEQ ID NO:122); miR-668 (SEQ ID NO:194), miR-122 (SEQ ID NO:22), miR-1227 (SEQ ID NO:157), miR-26a-2# (SEQ ID NO:82), miR-34a (SEQ ID NO:166), miR-551b# (SEQ ID NO:172), miR-1183 (SEQ ID NO:197) and miR-892b (SEQ ID NO:248), and combinations thereof;
(c) let-7b (SEQ ID NO:76), miR-106b# (SEQ ID NO:92), miR-1249 (SEQ ID NO:200), miR-145 (SEQ ID NO:207), miR-151-5P (SEQ ID NO:111), miR-154# (SEQ ID NO:139), miR-15a (SEQ ID NO:208), miR-181a (SEQ ID NO:66), miR-181c (SEQ ID NO:209), miR-18a# (SEQ ID NO:211), miR-194 (SEQ ID NO:115), miR-199a-5p (SEQ ID NO:81), miR-199b-5p (SEQ ID NO:213), miR-20a (SEQ ID NO:89), miR-23b (SEQ ID NO:222), miR-30e-3p (SEQ ID NO:224), miR-324-5p (SEQ ID NO:73), miR-411 (SEQ ID NO:147), miR-431 (SEQ ID NO:227), miR-487a (SEQ ID NO:231), miR-493 (SEQ ID NO:232), miR-494 (SEQ ID NO:233), miR-495 (SEQ ID NO:102), miR-505 (SEQ ID NO:235), miR-520a-3p (SEQ ID NO:188), miR-744# (SEQ ID NO:245), miR-769-5p (SEQ ID NO:246), let-7a# (SEQ ID NO:155), miR-10b# (SEQ ID NO:5), miR-1183 (SEQ ID NO:197), miR-1233 (SEQ ID NO:198), miR-1247 (SEQ ID NO:199), miR-1260 (SEQ ID NO:47), miR-1270 (SEQ ID NO:201), miR-1274A (SEQ ID NO:202), miR-1275 (SEQ ID NO:203), miR-128 (SEQ ID NO:158), miR-1290 (SEQ ID NO:34), miR-1298 (SEQ ID NO:204), miR-1303 (SEQ ID NO:3), miR-130a (SEQ ID NO:112), miR-135b (SEQ ID NO:205), miR-138 (SEQ ID NO:206), miR-140-5p (SEQ ID NO:160), miR-141 (SEQ ID NO:161), miR-142-5p (SEQ ID NO:43), miR-146b-5p (SEQ ID NO:24), miR-148a (SEQ ID NO:91), miR-150 (SEQ ID NO:27), miR-152 (SEQ ID NO:130), miR-15a# (SEQ ID NO:64), miR-185 (SEQ ID NO:163), miR-186 (SEQ ID NO:210), miR-193a-3p (SEQ ID NO.: 212), miR-193b (SEQ ID NO:12), miR-197 (SEQ ID NO:10), miR-19a (SEQ ID NO:74), miR-200a (SEQ ID NO:214), miR-205 (SEQ ID NO:215), miR-206 (SEQ ID NO:23), miR-20b (SEQ ID NO:216), miR-21 (SEQ ID NO:51), miR-21# (SEQ ID NO: 141), miR-214 (SEQ ID NO:217), miR-214# (SEQ ID NO:218), miR-218 (SEQ ID NO: 219), miR-220b (SEQ ID NO:220), miR-223 (SEQ ID NO:221), miR-30a-3p (SEQ ID NO: 223), miR-30a-5p (SEQ ID NO:164), miR-30d (SEQ ID NO:165), miR-320 (SEQ ID NO: 37), miR-324-3p (SEQ ID NO:107), miR-326 (SEQ ID NO:225), miR-335 (SEQ ID NO: 119), miR-338-5P (SEQ ID NO:15), miR-346 (SEQ ID NO:226), miR-34a# (SEQ ID NO: 13), miR-362-3p (SEQ ID NO:96), miR-375 (SEQ ID NO:8), miR-378 (SEQ ID NO:167), miR-425 (SEQ ID NO:168), miR-450a (SEQ ID NO:228), miR-450b-5p (SEQ ID NO:229), miR-455-5p (SEQ ID NO:230), miR-501-5p (SEQ ID NO:234) miR-511 (SEQ ID NO:236), miR-518d-3p (SEQ ID NO:237), miR-518e (SEQ ID NO:238), miR-518f (SEQ ID NO:239), miR-548a-3p (SEQ ID NO:14), miR-548c-3p (SEQ ID NO:240), miR-570 (SEQ ID NO:241), miR-571 (SEQ ID NO:242), miR-574-3p (SEQ ID NO:4), miR-577 (SEQ ID NO:243), miR-579 (SEQ ID NO:170), miR-618 (SEQ ID NO:244), miR-885-5p (SEQ ID NO:247), miR-9# (SEQ ID NO:249), miR-95 (SEQ ID NO:250), let-7a (SEQ ID NO:173), let-7d (SEQ ID NO:45), let-7e (SEQ ID NO:93), let-7f (SEQ ID NO:174), let-7g (SEQ ID NO:72); miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-379 (SEQ ID NO:186), miR-411 (SEQ ID NO:124), miR-454 (SEQ ID NO:187), miR-539 (SEQ ID NO:189), miR-543 (SEQ ID NO:133), miR-548J (SEQ ID NO:148), miR-664 (SEQ ID NO:152), miR-758 (SEQ ID NO:190), miR-766 (SEQ ID NO:191), miR-9 (SEQ ID NO:192), miR-98 (SEQ ID NO:193), miR-99b (SEQ ID NO:122); miR-668 (SEQ ID NO:194), miR-106a (SEQ ID NO:156), miR-122 (SEQ ID NO:22), miR-1227 (SEQ ID NO:157), miR-132 (SEQ ID NO:159), miR-146a (SEQ ID NO:21), miR-17 (SEQ ID NO:162), miR-222 (SEQ ID NO:16), miR-26a-2# (SEQ ID NO: 82), miR-345 (SEQ ID NO:18), miR-34a (SEQ ID NO:166), miR-429 (SEQ ID NO:169), miR-590-5p (SEQ ID NO:104), miR-598 (SEQ ID NO:110), miR-551b# (SEQ ID NO: 172), miR-1183 (SEQ ID NO:197) and miR-892b, (SEQ ID NO:248) and combinations thereof; and
(d) of miR-106b# (SEQ ID NO:92), miR-1249 (SEQ ID NO:200), miR-145 (SEQ ID NO:207), miR-199a-5p (SEQ ID NO:81), miR-23b (SEQ ID NO:222), miR-29b (SEQ ID NO:125), miR-431 (SEQ ID NO:227), miR-487a (SEQ ID NO:231), miR-493 (SEQ ID NO:232), miR-494 (SEQ ID NO:233), miR-495 (SEQ ID NO:102), miR-744 (SEQ ID NO:245), miR-154# (SEQ ID NO:139), miR-27b# (SEQ ID NO:180), miR-374a (SEQ ID NO:68), miR-411# (SEQ ID NO:147), miR-454 (SEQ ID NO:187), miR-520a-3p (SEQ ID NO:188), miR-548J (SEQ ID NO:148), miR-10b# (SEQ ID NO:5), miR-1183 (SEQ ID NO:197), miR-1233 (SEQ ID NO:198), miR-1247 (SEQ ID NO:199), miR-1260 (SEQ ID NO:47), miR-1270 (SEQ ID NO:201), miR-1274A (SEQ ID NO:202), miR-1275 (SEQ ID NO:203), miR-1290 (SEQ ID NO:34), miR-1298 (SEQ ID NO:204), miR-1303 (SEQ ID NO:3), miR-135b (SEQ ID NO:205), miR-138 (SEQ ID NO:206), miR-193a-3p (SEQ ID NO:212), miR-193b (SEQ ID NO:12), miR-197 (SEQ ID NO:10), miR-205 (SEQ ID NO:215), miR-206 (SEQ ID NO:23), miR-214# (SEQ ID NO:218), miR-214 (SEQ ID NO:217), miR-218 (SEQ ID NO:219), miR-220b (SEQ ID NO:220), miR-223 (SEQ ID NO:221), miR-338-5P (SEQ ID NO:15), miR-346 (SEQ ID NO:226), miR-34a# (SEQ ID NO:13), miR-375 (SEQ ID NO:8), miR-429 (SEQ ID NO:169), miR-450a (SEQ ID NO:228), miR-450b-5p (SEQ ID NO:229), miR-455-5p (SEQ ID NO:230), miR-501-5p (SEQ ID NO:234), miR-511 (SEQ ID NO:236), miR-518d-3p (SEQ ID NO:237), miR-518e (SEQ ID NO:238), miR-518f (SEQ ID NO:239), miR-548a-3p (SEQ ID NO:14), miR-548c-3p (SEQ ID NO:240), miR-570 (SEQ ID NO:241), miR-571 (SEQ ID NO:242), miR-577 (SEQ ID NO:243), miR-618 (SEQ ID NO:244), miR-885-5p (SEQ ID NO:247), miR-95 (SEQ ID NO:250), let-7a# (SEQ ID NO:155), miR-130a (SEQ ID NO:112), miR-132 (SEQ ID NO:159), miR-141 (SEQ ID NO:161), miR-148a (SEQ ID NO:91), miR-150 (SEQ ID NO:27), miR-345 (SEQ ID NO:18), miR-362-3p (SEQ ID NO:96), miR-378 (SEQ ID NO: 167), miR-579 (SEQ ID NO:170), miR-598 (SEQ ID NO:110), let-7a (SEQ ID NO:173), let-7d (SEQ ID NO:45), let-7e (SEQ ID NO:93), let-7f (SEQ ID NO:174), let-7g (SEQ ID NO:72); miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-379 (SEQ ID NO:186), miR-411 (SEQ ID NO:124), miR-454 (SEQ ID NO:187), miR-539 (SEQ ID NO:189), miR-543 (SEQ ID NO:133), miR-548J (SEQ ID NO:148), miR-664 (SEQ ID NO:152), miR-758 (SEQ ID NO:190), miR-766 (SEQ ID NO:191), miR-9 (SEQ ID NO:192), miR-98 (SEQ ID NO:193), miR-99b (SEQ ID NO:122); miR-668 (SEQ ID NO:194), miR-106a (SEQ ID NO:156), miR-122 (SEQ ID NO:22), miR-1227 (SEQ ID NO:157), miR-132 (SEQ ID NO:159), miR-146a (SEQ ID NO:21), miR-17 (SEQ ID NO:162), miR-222 (SEQ ID NO:16), miR-26a-2# (SEQ ID NO: 82), miR-345 (SEQ ID NO:18), miR-34a (SEQ ID NO:166), miR-429 (SEQ ID NO:169), miR-590-5p (SEQ ID NO:104), miR-598 (SEQ ID NO:110), miR-551b# (SEQ ID NO: 172), miR-1183 (SEQ ID NO:197) and miR-892b, (SEQ ID NO:248) and combinations thereof.

19-21. (canceled)

22. The method of claim 1, wherein the level or expression pattern of at least 2 different microRNAs is detected.

23-34. (canceled)

35. The method of claim 18, wherein the absence or a decreased level of one, two, three, four, five, six, seven or more microRNA is detected, relative to a predetermined criterion, is indicative of a diagnosis of IPF, and the one or more microRNAs is selected from the group consisting of
(a) let-7a (SEQ ID NO:173), let-7d (SEQ ID NO:45), let-7e (SEQ ID NO:93),
let-7f (SEQ ID NO:174), let-7g (SEQ ID NO:72), miR-103 (SEQ ID NO:105), miR-106b# (SEQ ID NO:92), miR-107 (SEQ ID NO:175), miR-1244 (SEQ ID NO:20), miR-1249 (SEQ ID NO:200), miR-125a-5p (SEQ ID NO:176), miR-127-3p (SEQ ID NO:101), miR-142-3p (SEQ ID NO:38), miR-144# (SEQ ID NO:69), miR-145 (SEQ ID NO:207), miR-148b# (SEQ ID NO:128), miR-151-5P (SEQ ID NO:111), miR-15a (SEQ ID NO:208), miR-15b (SEQ ID NO:56), miR-181a (SEQ ID NO:66), miR-181a-2# (SEQ ID NO:177), miR-181c (SEQ ID NO:209), miR-18a# (SEQ ID NO:211), miR-190 (SEQ ID NO:63), miR-194 (SEQ ID NO:115), miR-196b (SEQ ID NO:140), miR-199a-5p (SEQ ID NO:81), miR-199b-5p (SEQ ID NO:213), miR-19b-1# (SEQ ID NO:178), miR-200c (SEQ ID NO: 179), miR-20a (SEQ ID NO:89), miR-20a# (SEQ ID NO:75), miR-23b (SEQ ID NO:222), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO:42), miR-30c (SEQ ID NO:52), miR-30e-3p (SEQ ID NO:224), miR-324-5p (SEQ ID NO:73), miR-331-3p (SEQ ID NO:181), miR-339-5p (SEQ ID NO: 182), miR-340# (SEQ ID NO:127), miR-362-5p (SEQ ID NO:183), miR-370 (SEQ ID NO: 184), miR-374a (SEQ ID NO:68), miR-374b (SEQ ID NO:185), miR-379 (SEQ ID NO: 186), miR-411 (SEQ ID NO:124), miR-431 (SEQ ID NO:227), miR-454 (SEQ ID NO: 187), miR-487a (SEQ ID NO:231), miR-493 (SEQ ID NO:232), miR-494 (SEQ ID NO: 233), miR-495 (SEQ ID NO:102), miR-505# (SEQ ID NO:235), miR-539 (SEQ ID NO: 189), miR-543 (SEQ ID NO:133), miR-548J (SEQ ID NO:148), miR-664 (SEQ ID NO: 152), miR-744# (SEQ ID NO:245), miR-758 (SEQ ID NO:190), miR-766 (SEQ ID NO: 191), miR-769-5p (SEQ ID NO:246), miR-9 (SEQ ID NO:192), miR-98 (SEQ ID NO:193), miR-99b (SEQ ID NO:122), miR-148b# (SEQ ID NO:128), miR-668 (SEQ ID NO:194), miR-411# (SEQ ID NO:147), miR-520a-3p (SEQ ID NO. 188), and combinations thereof;
(b) let-7a (SEQ ID NO:173), let-7d (SEQ ID NO:45), let-7e (SEQ ID NO:93), let-7f (SEQ ID NO:174), miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-20a# (SEQ ID NO:75), miR-24-2# (SEQ ID NO:120), miR-26a (SEQ ID NO:70), miR-28-5p (SEQ ID NO:90), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-379 (SEQ ID NO:186), miR-411 (SEQ ID NO:124), miR-539 (SEQ ID NO:189), miR-543 (SEQ ID NO:133), miR-664 (SEQ ID NO:152), miR-758 (SEQ ID NO:190), miR-9 (SEQ ID NO:192), miR-98 (SEQ ID NO:193). miR-99b (SEQ ID NO:122); miR-668 (SEQ ID NO:194), and combinations thereof;
(c) let-7a (SEQ ID NO:173), let-7d (SEQ ID NO:45), let-7e (SEQ ID NO:93), let-7f (SEQ ID NO:174), let-7g (SEQ ID NO:72); miR-103(SEQ ID NO:105), miR-107 (SEQ ID NO:175), miR-1244(SEQ ID NO:20), miR-125a-5p (SEQ ID NO:176), miR-127-3p (SEQ ID NO:101), miR-142-3p (SEQ ID NO:38), miR-144# (SEQ ID NO:69), miR-148b# (SEQ ID NO:128), miR-15b (SEQ ID NO:56), miR-181a-2# (SEQ ID NO:177), miR-190 (SEQ ID NO:63), miR-196b (SEQ ID NO:140), miR-19b-1# (SEQ ID NO:178), miR-200c (SEQ ID NO:179), miR-20a# (SEQ ID NO:75), miR-24-2# (SEQ ID NO:120), miR-26a (SEQ ID NO:70), miR-27b# (SEQ ID NO:180), miR-28-5p (SEQ ID NO:90), miR-29b (SEQ ID NO:125), miR-301a (SEQ ID NO:67), miR-30b (SEQ ID NO:42), miR-30c (SEQ ID NO:52), miR-331-3p (SEQ ID NO:181), miR-339-5p (SEQ ID NO:182), miR-340# (SEQ ID NO:127), miR-362-5p (SEQ ID NO:183), miR-370 (SEQ ID NO:184), miR-374a (SEQ ID NO:68), miR-374b (SEQ ID NO:185), miR-379 (SEQ ID NO:186), miR-411 (SEQ ID NO:124), miR-454 (SEQ ID NO:187), miR-539 (SEQ ID NO:189), miR-543 (SEQ ID NO:133), miR-548J (SEQ ID NO:148), miR-664 (SEQ ID NO:152), miR-758 (SEQ ID NO:190), miR-766 (SEQ ID NO:191), miR-9 (SEQ ID NO:192), miR-98 (SEQ ID NO:193), miR-99b (SEQ ID NO:122); miR-668 (SEQ ID NO:194), and combinations thereof;
(d) miR-106b# (SEQ ID NO:92), miR-1249 (SEQ ID NO:200), miR-145 (SEQ ID NO:207), miR-199a-5p (SEQ ID NO:81), miR-23b (SEQ ID NO:222), miR-29b (SEQ ID NO:125), miR-431 (SEQ ID NO:227), miR-487a (SEQ ID NO:231), miR-493 (SEQ ID NO:232), miR-494 (SEQ ID NO:233), miR-495 (SEQ ID NO:102), miR-744# (SEQ ID NO:245), miR-154# (SEQ ID NO:139), miR-27b# (SEQ ID NO:180), miR-374a (SEQ ID NO:68), miR-411# (SEQ ID NO:147), miR-454 (SEQ ID NO:187), miR-520a-3p (SEQ ID NO:188), miR-548J (SEQ ID NO:148), and combinations thereof; and
(e) miR-106b# (SEQ ID NO:92), miR-1249 (SEQ ID NO:200), miR-145 (SEQ ID NO:207), miR-151-5P (SEQ ID NO:111), miR-154# (SEQ ID NO:139), miR-15a (SEQ ID NO:208), miR-181a (SEQ ID NO:66), miR-181c (SEQ ID NO:209), miR-18a# (SEQ ID NO:211), miR-194 (SEQ ID NO:115), miR-199a-5p (SEQ ID NO:81), miR-199b-5p (SEQ ID NO:213), miR-20a (SEQ ID NO:89), miR-23b (SEQ ID NO:222), miR-30e-3p (SEQ ID NO:224), miR-324-5p (SEQ ID NO:73), miR-411# (SEQ ID NO:147), miR-431 (SEQ ID NO:227), miR-487a (SEQ ID NO:231), miR-493 (SEQ ID NO:232), miR-494 (SEQ ID NO:233), miR-495 (SEQ ID NO:102), miR-505# (SEQ ID NO:235), miR-520a-3p (SEQ ID NO:188), miR-744# (SEQ ID NO:245), miR-769-5p (SEQ ID NO: 246), and combinations thereof.

36-39. (canceled)

40. The method of claim 18, wherein the presence or an increased level of one, two, three, four, five, six, seven or more microRNA is detected, relative to a predetermined criterion, is indicative of a diagnosis of IPF, and the one or more microRNAs is selected from the group consisting of
(a) let-7b (SEQ ID NO:76), miR-106a (SEQ ID NO:156), miR-10b# (SEQ ID NO:5), miR-1183 (SEQ ID NO:197), miR-122 (SEQ ID NO:22), miR-1227 (SEQ ID NO: 157), miR-1233 (SEQ ID NO:198), miR-1247 (SEQ ID NO:199), miR-1270 (SEQ ID NO: 201), miR-1274A (SEQ ID NO:202), miR-1275 (SEQ ID NO:203), miR-1290 (SEQ ID NO:34), miR-1298 (SEQ ID NO:204), miR-1303 (SEQ ID NO:3), miR-132 (SEQ ID NO: 159), miR-135b (SEQ ID NO:205), miR-138 (SEQ ID NO:206), miR-146a (SEQ ID NO: 21), miR-17 (SEQ ID NO:162), miR-186 (SEQ ID NO:210), miR-193a-3p (SEQ ID NO: 212), miR-193b (SEQ ID NO:12), miR-197 (SEQ ID NO:10), miR-200a (SEQ ID NO: 214), miR-205 (SEQ ID NO:215), miR-206 (SEQ ID NO:23), miR-20b (SEQ ID NO:216), miR-214 (SEQ ID NO:217), miR-214# (SEQ ID NO:218), miR-218 (SEQ ID NO:219), miR-220b (SEQ ID NO:220), miR-222 (SEQ ID NO:16), miR-223 (SEQ ID NO:221), miR-26a-2# (SEQ ID NO:82), miR-30a-3p (SEQ ID NO:223), miR-320 (SEQ ID NO:37), miR-326 (SEQ ID NO:225), miR-338-5P (SEQ ID NO:15), miR-345 (SEQ ID NO:18), miR-346 (SEQ ID NO:226), miR-34a (SEQ ID NO:166), miR-34a# (SEQ ID NO:13), miR-375 (SEQ ID NO:8), miR-429 (SEQ ID NO:169), miR-450a (SEQ ID NO:228), miR-450b-5p (SEQ ID NO:229), miR-455-5p (SEQ ID NO:230), miR-501-5p (SEQ ID NO:234), miR-511 (SEQ ID NO:236), miR-518d-3p (SEQ ID NO:237), miR-518e (SEQ ID NO: 238), miR-518f (SEQ ID NO:239), miR-548a-3p (SEQ ID NO:14), miR-548c-3p (SEQ ID NO:240), miR-570 (SEQ ID NO:241), miR-571 (SEQ ID NO:242), miR-574-3p (SEQ ID NO:4), miR-577 (SEQ ID NO:243), miR-590-5p (SEQ ID NO:104), miR-598 (SEQ ID NO:110), miR-618 (SEQ ID NO:244), miR-885-5p (SEQ ID NO:247), miR-9# (SEQ ID NO:249), miR-95 (SEQ ID NO:250), miR-26a-2# (SEQ ID NO:82), miR-551b# (SEQ ID NO:172), let-7a# (SEQ ID NO:155), miR-1260 (SEQ ID NO:47), miR-128 (SEQ ID NO: 158), miR-130a (SEQ ID NO:112), miR-140-5p (SEQ ID NO:160), miR-141 (SEQ ID NO: 161), miR-142-5p (SEQ ID NO:43), miR-146b-5p (SEQ ID NO:24), miR-148a (SEQ ID NO:91), miR-150 (SEQ ID NO:27), miR-152 (SEQ ID NO:130), miR-15a# (SEQ ID NO:64), miR-185 (SEQ ID NO:163), miR-19a (SEQ ID NO:74), miR-21 (SEQ ID NO:51), miR-21# (SEQ ID NO:141), miR-30a-5p (SEQ ID NO:164), miR-30d (SEQ ID NO:165), miR-324-3p (SEQ ID NO:107), miR-335 (SEQ ID NO:119), miR-362-3p (SEQ ID NO:96), miR-378 (SEQ ID NO:167), miR-425 (SEQ ID NO:168), miR-579 (SEQ ID NO:170), and combinations thereof;
(b) miR-122 (SEQ ID NO:22), miR-1227 (SEQ ID NO:157), miR-26a-2# (SEQ ID NO:82), miR-34a (SEQ ID NO:166), miR-551b# (SEQ ID NO:172), and combinations thereof;
(c) miR-106a (SEQ ID NO:156), miR-122 (SEQ ID NO:22), miR-1227 (SEQ ID NO:157), miR-132 (SEQ ID NO:159), miR-146a (SEQ ID NO:21), miR-17 (SEQ ID NO: 162), miR-222 (SEQ ID NO:16), miR-26a-2# (SEQ ID NO:82), miR-345 (SEQ ID NO: 18), miR-34a (SEQ ID NO:166), miR-429 (SEQ ID NO:169), miR-590-5p (SEQ ID NO: 104), miR-598 (SEQ ID NO:110), miR-551b# (SEQ ID NO:172), and combinations thereof;
(d) miR-10b# (SEQ ID NO:5), miR-1183 (SEQ ID NO:197), miR-1233 (SEQ ID NO:198), miR-1247 (SEQ ID NO:199), miR-1260 (SEQ ID NO:47), miR-1270 (SEQ ID NO:201), miR-1274A (SEQ ID NO:202), miR-1275 (SEQ ID NO:203), miR-1290 (SEQ ID NO:34), miR-1298 (SEQ ID NO:204), miR-1303 (SEQ ID NO:3), miR-135b (SEQ ID NO:205), miR-138 (SEQ ID NO:206), miR-193a-3p (SEQ ID NO:212), miR-193b (SEQ ID NO:12), miR-197 (SEQ ID NO:10), miR-205 (SEQ ID NO:215), miR-206 (SEQ ID NO:23), miR-214# (SEQ ID NO:218), miR-214 (SEQ ID NO:217), miR-218 (SEQ ID NO: 219), miR-220b (SEQ ID NO:220), miR-223 (SEQ ID NO:221), miR-338-5P (SEQ ID NO: 15), miR-346 (SEQ ID NO:226), miR-34a# (SEQ ID NO:13), miR-375 (SEQ ID NO:8), miR-429 (SEQ ID NO:169), miR-450a (SEQ ID NO:228), miR-450b-5p (SEQ ID NO:229), miR-455-5p (SEQ ID NO:230), miR-501-5p (SEQ ID NO:234), miR-511 (SEQ ID NO: 236), miR-518d-3p (SEQ ID NO:237), miR-518e (SEQ ID NO:238), miR-518f (SEQ ID NO:239), miR-548a-3p (SEQ ID NO:14), miR-548c-3p (SEQ ID NO:240), miR-570 (SEQ ID NO:241), miR-571 (SEQ ID NO:242), miR-577 (SEQ ID NO:243), miR-618 (SEQ ID NO:244), miR-885-5p (SEQ ID NO:247), miR-95 (SEQ ID NO:250), let-7a# (SEQ ID NO: 155), miR-130a (SEQ ID NO:112), miR-132 (SEQ ID NO:159), miR-141 (SEQ ID NO: 161), miR-148a (SEQ ID NO:91), miR-150 (SEQ ID NO:27), miR-345 (SEQ ID NO:18), miR-362-3p (SEQ ID NO:96), miR-378 (SEQ ID NO:167), miR-579 (SEQ ID NO:170), miR-598 (SEQ ID NO:110), and combinations thereof; and
(e) let-7b (SEQ ID NO:76), let-7a# (SEQ ID NO:155), miR-10b# (SEQ ID NO: 5), miR-1183 (SEQ ID NO:197), miR-1233 (SEQ ID NO:198), miR-1247 (SEQ ID NO: 199), miR-1260 (SEQ ID NO:47), miR-1270 (SEQ ID NO:201), miR-1274A (SEQ ID NO: 202), miR-1275 (SEQ ID NO:203), miR-128 (SEQ ID NO:158), miR-1290 (SEQ ID NO: 34), miR-1298 (SEQ ID NO:204), miR-1303 (SEQ ID NO:3), miR-130a (SEQ ID NO: 112), miR-135b (SEQ ID NO:205), miR-138 (SEQ ID NO:206), miR-140-5p (SEQ ID NO: 160), miR-141 (SEQ ID NO:161), miR-142-5p (SEQ ID NO:43), miR-146b-5p (SEQ ID NO:24), miR-148a (SEQ ID NO:91), miR-150 (SEQ ID NO:27), miR-152 (SEQ ID NO: 130), miR-15a# (SEQ ID NO:64), miR-185 (SEQ ID NO:163), miR-186 (SEQ ID NO:210), miR-193a-3p (SEQ ID NO:212), miR-193b (SEQ ID NO:12), miR-197 (SEQ ID NO:10), miR-19a (SEQ ID NO:74), miR-200a (SEQ ID NO:214), miR-205 (SEQ ID NO:215), miR-206 (SEQ ID NO:23), miR-20b (SEQ ID NO:216), miR-21 (SEQ ID NO:51), miR-21 (SEQ ID NO:141), miR-214 (SEQ ID NO:217), miR-214# (SEQ ID NO:218), miR-218 (SEQ ID NO:219), miR-220b (SEQ ID NO:220), miR-223 (SEQ ID NO:221), miR-30a-3p (SEQ ID NO:223), miR-30a-5p (SEQ ID NO:164), miR-30d (SEQ ID NO:165), miR-320 (SEQ ID NO:37), miR-324-3p (SEQ ID NO:107), miR-326 (SEQ ID NO:225), miR-335 (SEQ ID NO:119), miR-338-5P (SEQ ID NO:15), miR-346 (SEQ ID NO:226), miR-34a# (SEQ ID NO:13), miR-362-3p (SEQ ID NO:96), miR-375 (SEQ ID NO:8), miR-378 (SEQ ID NO:167), miR-425 (SEQ ID NO:168), miR-450a (SEQ ID NO:228), miR-450b-5p (SEQ ID NO:229), miR-455-5p (SEQ ID NO:230), miR-501-5p (SEQ ID NO:234) miR-511 (SEQ ID NO:236), miR-518d-3p (SEQ ID NO:237), miR-518e (SEQ ID NO:238), miR-518f (SEQ ID NO:239), miR-548a-3p (SEQ ID NO:14), miR-548c-3p (SEQ ID NO:240), miR-570 (SEQ ID NO:241), miR-571 (SEQ ID NO:242), miR-574-3p (SEQ ID NO:4), miR-577 (SEQ ID NO:243), miR-579 (SEQ ID NO:170), miR-618 (SEQ ID NO:244), miR-885-5p (SEQ ID NO:247), miR-9# (SEQ ID NO:249), miR-95 (SEQ ID NO:250) and combinations thereof.

41-44. (canceled)

45. The method of claim 18, wherein the absence or a decreased level of one, two, three, four, five, six, seven or more microRNA is detected, relative to a predetermined criterion, is indicative of a diagnosis of progressive IPF, and the one or more microRNAs is selected from the group consisting of miR-1183 (SEQ ID NO:197) and miR-892b (SEQ ID NO:248).

46. The method of claim 1, wherein the sample is selected from the group consisting of whole blood, serum, plasma, exosomes and isolated microvesicles.

47. The method of claim 1, wherein the method further comprises administering a therapeutic agent to the subject.

48. A method of treating a human subject diagnosed with idiopathic pulmonary fibrosis (IPF) according to claim 1 comprising administering a therapeutic agent to the subject to treat IPF.

49. A method of treating a human subject identified as having idiopathic pulmonary fibrosis (IPF) or at risk of IPF based on an abnormal level of one, two, three, four, five, six, seven or more IPF-associated microRNAs in a blood sample of the subject, comprising administering a therapeutic agent to the subject to treat IPF.

50-53. (canceled)

54. The method of claim 49, wherein the sample is selected from the group consisting of whole blood, serum, plasma, exosomes and isolated microvesicles.

55. The method of claim 48, wherein the therapeutic agent is an oligonucleotide that decreases the activity or level of expression of one, two, three, four, five, six, seven or more of the microRNA in the subject.

56. The method of claim 48, wherein the therapeutic agent is an oligonucleotide that increases the activity or level of expression of one, two, three, four, five, six, seven or more of the microRNA in the subject.

57. The method of claim 48, wherein the therapeutic agent is an anti-fibrotic agent.

58. The method of claim 57, wherein the anti-fibrotic agent is pirfenidone.

59. (canceled)

60. A kit to be used in the diagnosis of subjects having idiopathic pulmonary fibrosis (IPF) comprising one, two, three, four, five, six, seven or more probes that specifically hybridize to, or primers that specifically amplify, one or more microRNAs selected from the group consisting of miR-155 (SEQ ID NO:1), miR-767-3p (SEQ ID NO:2), miR-1303 (SEQ ID NO:3), miR-574-3p (SEQ ID NO:4), miR-10b# (SEQ ID NO:5), miR-875-5p (SEQ ID NO:6), miR-29a (SEQ ID NO:7), miR-375 (SEQ ID NO:8), miR-342-3p (SEQ ID NO:9), miR-197 (SEQ ID NO:10), miR-663B (SEQ ID NO:11), miR-193b (SEQ ID NO:12), miR-34a# (SEQ ID NO:13), miR-548a-3p (SEQ ID NO:14), miR-338-5P (SEQ ID NO:15), miR-222 (SEQ ID NO:16), miR-520D-3P (SEQ ID NO:17), miR-345 (SEQ ID NO:18), miR-99b# (SEQ ID NO:19), miR-1244 (SEQ ID NO:20), miR-146a (SEQ ID NO:21), miR-122 (SEQ ID NO:22), miR-206 (SEQ ID NO:23), miR-146b-5p (SEQ ID NO:24), miR-1300 (SEQ ID NO:25), miR-28-3p (SEQ ID NO:26), miR-150 (SEQ ID NO:27), miR-202 (SEQ ID NO:28), miR-636 (SEQ ID NO:29), miR-27a# (SEQ ID NO:30), miR-323-3p (SEQ ID NO:31), miR-520c-3p (SEQ ID NO:32), miR-191 (SEQ ID NO:33), miR-1290 (SEQ ID NO:34), miR-572 (SEQ ID NO:35), miR-886-3p (SEQ ID NO:36), miR-320 (SEQ ID NO:37), miR-142-3p (SEQ ID NO:38), miR-18a (SEQ ID NO:39), miR-26b (SEQ ID NO:40), miR-106b (SEQ ID NO:41), miR-30b (SEQ ID NO:42), miR-142-5p (SEQ ID NO:43), miR-29c (SEQ ID NO:44), let-7d (SEQ ID NO:45), miR-144 (SEQ ID NO:46), miR-1260 (SEQ ID NO:47), miR-361-5p (SEQ ID NO:48), miR-520e (SEQ ID NO:49), miR-660 (SEQ ID NO:50), miR-21 (SEQ ID NO:51), miR-30c (SEQ ID NO:52), miR-148b (SEQ ID NO:53), miR-27b (SEQ ID NO:54), miR-15b# (SEQ ID NO:55), miR-15b (SEQ ID NO:56), miR-16-1# (SEQ ID NO:57), miR-17# (SEQ ID NO:58), miR-22 (SEQ ID NO:59), miR-32 (SEQ ID NO:60), miR-532-5p (SEQ ID NO:61), miR-101 (SEQ ID NO:62), miR-190 (SEQ ID NO:63), miR-15a# (SEQ ID NO:64), miR-27a (SEQ ID NO:65), miR-181a (SEQ ID NO:66), miR-301a (SEQ ID NO:67), miR-374a (SEQ ID NO:68), miR-144# (SEQ ID NO:69), miR-26a (SEQ ID NO:70), miR-320B (SEQ ID NO:71), let-7g (SEQ ID NO:72), miR-324-5p (SEQ ID NO:73), miR-19a (SEQ ID NO:74), miR-20a# (SEQ ID NO:75), let-7b (SEQ ID NO:76), miR-422a (SEQ ID NO:77), let-7f-2# (SEQ ID NO:78), let-7g (SEQ ID NO:79), miR-128a (SEQ ID NO:80), miR-199a-5p (SEQ ID NO:81), miR-26a-2# (SEQ ID NO:82), miR-29a# (SEQ ID NO:83), miR-329 (SEQ ID NO:84), miR-337-5p (SEQ ID NO:85), miR-369-3p (SEQ ID NO:86), miR-376a# (SEQ ID NO:87), miR-486-3p (SEQ ID NO:88), miR-20a (SEQ ID NO:89), miR-28-5p (SEQ ID NO:90), miR-148a (SEQ ID NO:91), miR-106b# (SEQ ID NO:92), let-7e (SEQ ID NO:93), miR-25 (SEQ ID NO:94), miR-656 (SEQ ID NO:95), miR-362-3p (SEQ ID NO:96), miR-340 (SEQ ID NO:97), miR-451 (SEQ ID NO:98), miR-423-5p (SEQ ID NO:99), miR-652 (SEQ ID NO:100), miR-127-3p (SEQ ID NO:101), miR-495 (SEQ ID NO:102), miR-328 (SEQ ID NO:103), miR-590-5p (SEQ ID NO:104), miR-103 (SEQ ID NO:105), miR-19b (SEQ ID NO:106), miR-324-3p (SEQ ID NO:107), miR-145# (SEQ ID NO:108), miR-199a-3p (SEQ ID NO:109), miR-598 (SEQ ID NO:110), miR-151-5P (SEQ ID NO:111), miR-130a (SEQ ID NO:112), miR-502-3p (SEQ ID NO:113), miR-136# (SEQ ID NO:114), miR-194 (SEQ ID NO:115), miR-221 (SEQ ID NO:116), miR-22# (SEQ ID NO:117), miR-93 (SEQ ID NO:118), miR-335 (SEQ ID NO:119), miR-24-2# (SEQ ID NO:120), miR-130b (SEQ ID NO:121), miR-99b (SEQ ID NO:122), miR-195 (SEQ ID NO:123), miR-411 (SEQ ID NO:124), miR-29b (SEQ ID NO:125), miR-576-3p (SEQ ID NO:126), miR-340# (SEQ ID NO:127), miR-148b# (SEQ ID NO:128), miR-212 (SEQ ID NO:129), miR-152 (SEQ ID NO:130), miR-143 (SEQ ID NO:131), miR-7 (SEQ ID NO:132), miR-543 (SEQ ID NO:133), miR-30d (SEQ ID NO:134), miR-213 (SEQ ID NO:135), miR-126# (SEQ ID NO:136), miR-1197 (SEQ ID NO:137), miR-1255B (SEQ ID NO:138), miR-154# (SEQ ID NO:139), miR-196b (SEQ ID NO:140), miR-21# (SEQ ID NO:141), miR-335# (SEQ ID NO:142), miR-33a# (SEQ ID NO:143), miR-374a# (SEQ ID NO:144), miR-381 (SEQ ID NO:145), miR-409-5p (SEQ ID NO:146), miR-411# (SEQ ID NO:147), miR-548J (SEQ ID NO:148), miR-551b (SEQ ID NO:149), miR-616 (SEQ ID NO:150), miR-638 (SEQ ID NO:151), miR-664 (SEQ ID NO:152), miR-889 (SEQ ID NO:153), miR-29c# (SEQ ID NO:154), let-7a# (SEQ ID NO: 155), miR-106a (SEQ ID NO:156), miR-1227 (SEQ ID NO:157), miR-128 (SEQ ID NO: 158), miR-132 (SEQ ID NO:159), miR-140-5p (SEQ ID NO:160), miR-141 (SEQ ID NO: 161), miR-17 (SEQ ID NO:162), miR-185 (SEQ ID NO:163), miR-30a-5p (SEQ ID NO: 164), miR-30d (SEQ ID NO:165), miR-34a (SEQ ID NO:166), miR-378 (SEQ ID NO: 167), miR-425 (SEQ ID NO:168), miR-429 (SEQ ID NO:169), miR-579 (SEQ ID NO: 170), miR-523 (SEQ ID NO:171), miR-551b# (SEQ ID NO:172), let-7a (SEQ ID NO:173), let-7f (SEQ ID NO:174), miR-107 (SEQ ID NO:175), miR-125a-5p (SEQ ID NO:176), miR-181a-2# (SEQ ID NO:177), miR-19b-1# (SEQ ID NO:178), miR-200c (SEQ ID NO: 179), miR-27b# (SEQ ID NO:180), miR-331-3p (SEQ ID NO:181), miR-339-5p (SEQ ID NO:182), miR-362-5p (SEQ ID NO:183), miR-370 (SEQ ID NO:184), miR-374b (SEQ ID NO:185), miR-379 (SEQ ID NO:186), miR-454 (SEQ ID NO:187), miR-520a-3p (SEQ ID NO:188), miR-539 (SEQ ID NO:189), miR-758 (SEQ ID NO:190), miR-766 (SEQ ID NO: 191), miR-9 (SEQ ID NO:192), miR-98 (SEQ ID NO:193), miR-668 (SEQ ID NO:194), miR-1256 (SEQ ID NO:195), miR-299-5p (SEQ ID NO:196), miR-1183 (SEQ ID NO:197), miR-1233 (SEQ ID NO:198), miR-1247 (SEQ ID NO:199), miR-1249 (SEQ ID NO:200), miR-1270 (SEQ ID NO:201), miR-1274A (SEQ ID NO:202), miR-1275 (SEQ ID NO: 203), miR-1298 (SEQ ID NO:204), miR-135b (SEQ ID NO:205), miR-138 (SEQ ID NO: 206), miR-145 (SEQ ID NO:207), miR-15a (SEQ ID NO:208), miR-181c (SEQ ID NO: 209), miR-186 (SEQ ID NO:210), miR-18a# (SEQ ID NO:211), miR-193a-3p (SEQ ID NO:212), miR-199b-5p (SEQ ID NO:213), miR-200a (SEQ ID NO:214), miR-205 (SEQ ID NO:215), miR-20b (SEQ ID NO:216), miR-214 (SEQ ID NO:217), miR-214# (SEQ ID NO:218), miR-218 (SEQ ID NO:219), miR-220b (SEQ ID NO:220), miR-223 (SEQ ID NO:221), miR-23b (SEQ ID NO:222), miR-30a-3p (SEQ ID NO:223), miR-30e-3p (SEQ ID NO:224), miR-326 (SEQ ID NO:225), miR-346 (SEQ ID NO:226), miR-431 (SEQ ID NO:227), miR-450a (SEQ ID NO:228), miR-450b-5p (SEQ ID NO:229), miR-455-5p (SEQ ID NO:230), miR-487a (SEQ ID NO:231), miR-493 (SEQ ID NO:232), miR-494 (SEQ ID NO:233), miR-501-5p (SEQ ID NO:234), miR-505# (SEQ ID NO:235), miR-511 (SEQ ID NO:236), miR-518d-3p (SEQ ID NO:237), miR-518e (SEQ ID NO:238), miR-518f (SEQ ID NO:239), miR-548c-3p (SEQ ID NO:240), miR-570 (SEQ ID NO:241), miR-571 (SEQ ID NO:242), miR-577 (SEQ ID NO:243), miR-618 (SEQ ID NO:244), miR-744# (SEQ ID NO:245), miR-769-5p (SEQ ID NO:246), miR-885-5p (SEQ ID NO: 247), miR-892b (SEQ ID NO:248), miR-9# (SEQ ID NO:249), miR-95 (SEQ ID NO:250), and combinations thereof.

61. A diagnostic test system adapted for performing any of the methods of claim 1.

62. (canceled)

63. A computer program product comprising computer-executable instructions embodied in a computer-readable medium for performing the steps of any of the methods of claim 1.


SEQUENCE LISTING (250 sequences)

SEQ ID NO: 1 - RNA, 23 nt, Homo sapiens
uuaaugcuaaucgugauaggggu

SEQ ID NO: 2 - RNA, 23 nt, Homo sapiens
ucugcucauaccccaugguuucu

SEQ ID NO: 3 - RNA, 22 nt, Homo sapiens
uuuagagacggggucuugcucu

SEQ ID NO: 4 - RNA, 22 nt, Homo sapiens
cacgcucaugcacacacccaca

SEQ ID NO: 5 - RNA, 22 nt, Homo sapiens
acagauucgauucuaggggaau

SEQ ID NO: 6 - RNA, 22 nt, Homo sapiens
uauaccucaguuuuaucaggug

SEQ ID NO: 7 - RNA, 22 nt, Homo sapiens
uagcaccaucugaaaucgguua

SEQ ID NO: 8 - RNA, 22 nt, Homo sapiens
uuuguucguucggcucgcguga

SEQ ID NO: 9 - RNA, 23 nt, Homo sapiens
ucucacacagaaaucgcacccgu

SEQ ID NO: 10 - RNA, 22 nt, Homo sapiens
uucaccaccuucuccacccagc

SEQ ID NO: 11 - RNA, 22 nt, Homo sapiens
gguggcccggccgugccugagg

SEQ ID NO: 12 - RNA, 22 nt, Homo sapiens
aacuggcccucaaagucccgcu

SEQ ID NO: 13 - RNA, 22 nt, Homo sapiens
caaucagcaaguauacugcccu

SEQ ID NO: 14 - RNA, 22 nt, Homo sapiens
caaaacuggcaauuacuuuugc

SEQ ID NO: 15 - RNA, 22 nt, Homo sapiens
aacaauauccuggugcugagug

SEQ ID NO: 16 - RNA, 21 nt, Homo sapiens
agcuacaucuggcuacugggu

SEQ ID NO: 17 - RNA, 22 nt, Homo sapiens
aaagugcuucucuuuggugggu

SEQ ID NO: 18 - RNA, 22 nt, Homo sapiens
gcugacuccuaguccagggcuc

SEQ ID NO: 19 - RNA, 22 nt, Homo sapiens
caagcucgugucuguggguccg

SEQ ID NO: 20 - RNA, 26 nt, Homo sapiens
aaguaguugguuuguaugagaugguu

SEQ ID NO: 21 - RNA, 22 nt, Homo sapiens
ugagaacugaauuccauggguu

SEQ ID NO: 22 - RNA, 22 nt, Homo sapiens
uggagugugacaaugguguuug

SEQ ID NO: 23 - RNA, 22 nt, Homo sapiens
uggaauguaaggaagugugugg

SEQ ID NO: 24 - RNA, 22 nt, Homo sapiens
ugagaacugaauuccauaggcu

SEQ ID NO: 25 - RNA, 18 nt, Homo sapiens
uugagaaggaggcugcug

SEQ ID NO: 26 - RNA, 22 nt, Homo sapiens
cacuagauugugagcuccugga

SEQ ID NO: 27 - RNA, 22 nt, Homo sapiens
ucucccaacccuuguaccagug

SEQ ID NO: 28 - RNA, 20 nt, Homo sapiens
agagguauagggcaugggaa

SEQ ID NO: 29 - RNA, 23 nt, Homo sapiens
ugugcuugcucgucccgcccgca

SEQ ID NO: 30 - RNA, 22 nt, Homo sapiens
agggcuuagcugcuugugagca

SEQ ID NO: 31 - RNA, 21 nt, Homo sapiens
cacauuacacggucgaccucu

SEQ ID NO: 32 - RNA, 22 nt, Homo sapiens
aaagugcuuccuuuuagagggu

SEQ ID NO: 33 - RNA, 23 nt, Homo sapiens
caacggaaucccaaaagcagcug

SEQ ID NO: 34 - RNA, 19 nt, Homo sapiens
uggauuuuuggaucaggga

SEQ ID NO: 35 - RNA, 20 nt, Homo sapiens
guccgcucggcgguggccca

SEQ ID NO: 36 - RNA, 21 nt, Homo sapiens
cgcgggugcuuacugacccuu

SEQ ID NO: 37 - RNA, 22 nt, Homo sapiens
aaaagcuggguugagagggcga

SEQ ID NO: 38 - RNA, 23 nt, Homo sapiens
uguaguguuuccuacuuuaugga

SEQ ID NO: 39 - RNA, 23 nt, Homo sapiens
uaaggugcaucuagugcagauag

SEQ ID NO: 40 - RNA, 21 nt, Homo sapiens
uucaaguaauucaggauaggu

SEQ ID NO: 41 - RNA, 21 nt, Homo sapiens
uaaagugcugacagugcagau

SEQ ID NO: 42 - RNA, 22 nt, Homo sapiens
uguaaacauccuacacucagcu

SEQ ID NO: 43 - RNA, 21 nt, Homo sapiens
cauaaaguagaaagcacuacu

SEQ ID NO: 44 - RNA, 22 nt, Homo sapiens
uagcaccauuugaaaucgguua

SEQ ID NO: 45 - RNA, 22 nt, Homo sapiens
agagguaguagguugcauaguu

SEQ ID NO: 46 - RNA, 20 nt, Homo sapiens
uacaguauagaugauguacu

SEQ ID NO: 47 - RNA, 18 nt, Homo sapiens
aucccaccucugccacca

SEQ ID NO: 48 - RNA, 22 nt, Homo sapiens
uuaucagaaucuccagggguac

SEQ ID NO: 49 - RNA, 21 nt, Homo sapiens
aaagugcuuccuuuuugaggg

SEQ ID NO: 50 - RNA, 22 nt, Homo sapiens
uacccauugcauaucggaguug

SEQ ID NO: 51 - RNA, 22 nt, Homo sapiens
uagcuuaucagacugauguuga

SEQ ID NO: 52 - RNA, 23 nt, Homo sapiens
uguaaacauccuacacucucagc

SEQ ID NO: 53 - RNA, 22 nt, Homo sapiens
ucagugcaucacagaacuuugu

SEQ ID NO: 54 - RNA, 21 nt, Homo sapiens
uucacaguggcuaaguucugc

SEQ ID NO: 55 - RNA, 22 nt, Homo sapiens
cgaaucauuauuugcugcucua

SEQ ID NO: 56 - RNA, 22 nt, Homo sapiens
uagcagcacaucaugguuuaca

SEQ ID NO: 57 - RNA, 22 nt, Homo sapiens
ccaguauuaacugugcugcuga

SEQ ID NO: 58 - RNA, 22 nt, Homo sapiens
acugcagugaaggcacuuguag

SEQ ID NO: 59 - RNA, 22 nt, Homo sapiens
aagcugccaguugaagaacugu

SEQ ID NO: 60 - RNA, 22 nt, Homo sapiens
uauugcacauuacuaaguugca

SEQ ID NO: 61 - RNA, 22 nt, Homo sapiens
caugccuugaguguaggaccgu

SEQ ID NO: 62 - RNA, 21 nt, Homo sapiens
uacaguacugugauaacugaa

SEQ ID NO: 63 - RNA, 22 nt, Homo sapiens
ugauauguuugauauauuaggu

SEQ ID NO: 64 - RNA, 22 nt, Homo sapiens
caggccauauugugcugccuca

SEQ ID NO: 65 - RNA, 21 nt, Homo sapiens
uucacaguggcuaaguuccgc

SEQ ID NO: 66 - RNA, 23 nt, Homo sapiens
aacauucaacgcugucggugagu

SEQ ID NO: 67 - RNA, 23 nt, Homo sapiens
cagugcaauaguauugucaaagc

SEQ ID NO: 68 - RNA, 22 nt, Homo sapiens
uuauaauacaaccugauaagug

SEQ ID NO: 69 - RNA, 22 nt, Homo sapiens
ggauaucaucauauacuguaag

SEQ ID NO: 70 - RNA, 22 nt, Homo sapiens
uucaaguaauccaggauaggcu

SEQ ID NO: 71 - RNA, 22 nt, Homo sapiens
aaaagcuggguugagagggcaa

SEQ ID NO: 72 - RNA, 22 nt, Homo sapiens
ugagguaguaguuuguacaguu

SEQ ID NO: 73 - RNA, 23 nt, Homo sapiens
cgcauccccuagggcauuggugu

SEQ ID NO: 74 - RNA, 23 nt, Homo sapiens
ugugcaaaucuaugcaaaacuga

SEQ ID NO: 75 - RNA, 22 nt, Homo sapiens
acugcauuaugagcacuuaaag

SEQ ID NO: 76 - RNA, 22 nt, Homo sapiens
ugagguaguagguugugugguu

SEQ ID NO: 77 - RNA, 22 nt, Homo sapiens
acuggacuuagggucagaaggc

SEQ ID NO: 78 - RNA, 22 nt, Homo sapiens
cuauacagucuacugucuuucc

SEQ ID NO: 79 - RNA, 21 nt, Homo sapiens
cuguacaggccacugccuugc

SEQ ID NO: 80 - RNA, 21 nt, Homo sapiens
ucacagugaaccggucucuuu

SEQ ID NO: 81 - RNA, 23 nt, Homo sapiens
cccaguguucagacuaccuguuc

SEQ ID NO: 82 - RNA, 22 nt, Homo sapiens
ccuauucuugauuacuuguuuc

SEQ ID NO: 83 - RNA, 22 nt, Homo sapiens
acugauuucuuuugguguucag

SEQ ID NO: 84 - RNA, 22 nt, Homo sapiens
aacacaccugguuaaccucuuu

SEQ ID NO: 85 - RNA, 21 nt, Homo sapiens
gaacggcuucauacaggaguu

SEQ ID NO: 86 - RNA, 21 nt, Homo sapiens
aauaauacaugguugaucuuu

SEQ ID NO: 87 - RNA, 22 nt, Homo sapiens
guagauucuccuucuaugagua

SEQ ID NO: 88 - RNA, 21 nt, Homo sapiens
cggggcagcucaguacaggau

SEQ ID NO: 89 - RNA, 23 nt, Homo sapiens
uaaagugcuuauagugcagguag

SEQ ID NO: 90 - RNA, 22 nt, Homo sapiens
aaggagcucacagucuauugag

SEQ ID NO: 91 - RNA, 22 nt, Homo sapiens
ucagugcacuacagaacuuugu

SEQ ID NO: 92 - RNA, 22 nt, Homo sapiens
ccgcacuguggguacuugcugc

SEQ ID NO: 93 - RNA, 22 nt, Homo sapiens
ugagguaggagguuguauaguu

SEQ ID NO: 94 - RNA, 22 nt, Homo sapiens
cauugcacuugucucggucuga

SEQ ID NO: 95 - RNA, 21 nt, Homo sapiens
aauauuauacagucaaccucu

SEQ ID NO: 96 - RNA, 22 nt, Homo sapiens
aacacaccuauucaaggauuca

SEQ ID NO: 97 - RNA, 22 nt, Homo sapiens
uuauaaagcaaugagacugauu

SEQ ID NO: 98 - RNA, 22 nt, Homo sapiens
aaaccguuaccauuacugaguu

SEQ ID NO: 99 - RNA, 23 nt, Homo sapiens
ugaggggcagagagcgagacuuu

SEQ ID NO: 100 - RNA, 21 nt, Homo sapiens
aauggcgccacuaggguugug

SEQ ID NO: 101 - RNA, 22 nt, Homo sapiens
ucggauccgucugagcuuggcu

SEQ ID NO: 102 - RNA, 22 nt, Homo sapiens
aaacaaacauggugcacuucuu

SEQ ID NO: 103 - RNA, 22 nt, Homo sapiens
cuggcccucucugcccuuccgu

SEQ ID NO: 104 - RNA, 22 nt, Homo sapiens
gagcuuauucauaaaagugcag

SEQ ID NO: 105 - RNA, 23 nt, Homo sapiens
agcagcauuguacagggcuauga

SEQ ID NO: 106 - RNA, 23 nt, Homo sapiens
ugugcaaauccaugcaaaacuga

SEQ ID NO: 107 - RNA, 20 nt, Homo sapiens
acugccccaggugcugcugg

SEQ ID NO: 108 - RNA, 22 nt, Homo sapiens
ggauuccuggaaauacuguucu

SEQ ID NO: 109 - RNA, 22 nt, Homo sapiens
acaguagucugcacauugguua

SEQ ID NO: 110 - RNA, 22 nt, Homo sapiens
uacgucaucguugucaucguca

SEQ ID NO: 111 - RNA, 21 nt, Homo sapiens
ucgaggagcucacagucuagu

SEQ ID NO: 112 - RNA, 22 nt, Homo sapiens
cagugcaauguuaaaagggcau

SEQ ID NO: 113 - RNA, 22 nt, Homo sapiens
aaugcaccugggcaaggauuca

SEQ ID NO: 114 - RNA, 22 nt, Homo sapiens
caucaucgucucaaaugagucu

SEQ ID NO: 115 - RNA, 22 nt, Homo sapiens
uguaacagcaacuccaugugga

SEQ ID NO: 116 - RNA, 23 nt, Homo sapiens
agcuacauugucugcuggguuuc

SEQ ID NO: 117 - RNA, 22 nt, Homo sapiens
aguucuucaguggcaagcuuua

SEQ ID NO: 118 - RNA, 23 nt, Homo sapiens
caaagugcuguucgugcagguag

SEQ ID NO: 119 - RNA, 23 nt, Homo sapiens
ucaagagcaauaacgaaaaaugu

SEQ ID NO: 120 - RNA, 22 nt, Homo sapiens
ugccuacugagcugaaacacag

SEQ ID NO: 121 - RNA, 22 nt, Homo sapiens
cagugcaaugaugaaagggcau

SEQ ID NO: 122 - RNA, 22 nt, Homo sapiens
cacccguagaaccgaccuugcg

SEQ ID NO: 123 - RNA, 21 nt, Homo sapiens
uagcagcacagaaauauuggc

SEQ ID NO: 124 - RNA, 21 nt, Homo sapiens
uaguagaccguauagcguacg

SEQ ID NO: 125 - RNA, 23 nt, Homo sapiens
uagcaccauuugaaaucaguguu

SEQ ID NO: 126 - RNA, 22 nt, Homo sapiens
aagauguggaaaaauuggaauc

SEQ ID NO: 127 - RNA, 22 nt, Homo sapiens
uccgucucaguuacuuuauagc

SEQ ID NO: 128 - RNA, 22 nt, Homo sapiens
aaguucuguuauacacucaggc

SEQ ID NO: 129 - RNA, 21 nt, Homo sapiens
uaacagucuccagucacggcc

SEQ ID NO: 130 - RNA, 21 nt, Homo sapiens
ucagugcaugacagaacuugg

SEQ ID NO: 131 - RNA, 21 nt, Homo sapiens
ugagaugaagcacuguagcuc

SEQ ID NO: 132 - RNA, 23 nt, Homo sapiens
uggaagacuagugauuuuguugu

SEQ ID NO: 133 - RNA, 22 nt, Homo sapiens
aaacauucgcggugcacuucuu

SEQ ID NO: 134 - RNA, 22 nt, Homo sapiens
cuuucagucagauguuugcugc

SEQ ID NO: 135 - RNA, 22 nt, Homo sapiens
accaucgaccguugauuguacc

SEQ ID NO: 136 - RNA, 21 nt, Homo sapiens
cauuauuacuuuugguacgcg

SEQ ID NO: 137 - RNA, 21 nt, Homo sapiens
uaggacacauggucuacuucu

SEQ ID NO: 138 - RNA, 22 nt, Homo sapiens
cggaugagcaaagaaagugguu

SEQ ID NO: 139 - RNA, 22 nt, Homo sapiens
aaucauacacgguugaccuauu

SEQ ID NO: 140 - RNA, 22 nt, Homo sapiens
uagguaguuuccuguuguuggg

SEQ ID NO: 141 - RNA, 21 nt, Homo sapiens
caacaccagucgaugggcugu

SEQ ID NO: 142 - RNA, 22 nt, Homo sapiens
uuuuucauuauugcuccugacc

SEQ ID NO: 143 - RNA, 22 nt, Homo sapiens
caauguuuccacagugcaucac

SEQ ID NO: 144 - RNA, 22 nt, Homo sapiens
cuuaucagauuguauuguaauu

SEQ ID NO: 145 - RNA, 22 nt, Homo sapiens
uauacaagggcaagcucucugu

SEQ ID NO: 146 - RNA, 23 nt, Homo sapiens
agguuacccgagcaacuuugcau

SEQ ID NO: 147 - RNA, 22 nt, Homo sapiens
uauguaacacgguccacuaacc

SEQ ID NO: 148 - RNA, 22 nt, Homo sapiens
aaaaguaauugcggucuuuggu

SEQ ID NO: 149 - RNA, 21 nt, Homo sapiens
gcgacccauacuugguuucag

SEQ ID NO: 150 - RNA, 22 nt, Homo sapiens
agucauuggaggguuugagcag

SEQ ID NO: 151 - RNA, 25 nt, Homo sapiens
agggaucgcgggcggguggcggccu

SEQ ID NO: 152 - RNA, 23 nt, Homo sapiens
uauucauuuauccccagccuaca

SEQ ID NO: 153 - RNA, 21 nt, Homo sapiens
uuaauaucggacaaccauugu

SEQ ID NO: 154 - RNA, 22 nt, Homo sapiens
ugaccgauuucuccugguguuc

SEQ ID NO: 155 - RNA, 21 nt, Homo sapiens
cuauacaaucuacugucuuuc

SEQ ID NO: 156 - RNA, 23 nt, Homo sapiens
aaaagugcuuacagugcagguag

SEQ ID NO: 157 - RNA, 20 nt, Homo sapiens
cgugccacccuuuuccccag

SEQ ID NO: 158 - RNA, 21 nt, Homo sapiens
ucacagugaaccggucucuuu

SEQ ID NO: 159 - RNA, 22 nt, Homo sapiens
uaacagucuacagccauggucg

SEQ ID NO: 160 - RNA, 22 nt, Homo sapiens
cagugguuuuacccuaugguag

SEQ ID NO: 161 - RNA, 22 nt, Homo sapiens
uaacacugucugguaaagaugg

SEQ ID NO: 162 - RNA, 23 nt, Homo sapiens
caaagugcuuacagugcagguag

SEQ ID NO: 163 - RNA, 22 nt, Homo sapiens
uggagagaaaggcaguuccuga

SEQ ID NO: 164 - RNA, 22 nt, Homo sapiens
uguaaacauccucgacuggaag

SEQ ID NO: 165 - RNA, 22 nt, Homo sapiens
uguaaacauccccgacuggaag

SEQ ID NO: 166 - RNA, 22 nt, Homo sapiens
uggcagugucuuagcugguugu

SEQ ID NO: 167 - RNA, 21 nt, Homo sapiens
acuggacuuggagucagaagg

SEQ ID NO: 168 - RNA, 23 nt, Homo sapiens
aaugacacgaucacucccguuga

SEQ ID NO: 169 - RNA, 22 nt, Homo sapiens
uaauacugucugguaaaaccgu

SEQ ID NO: 170 - RNA, 23 nt, Homo sapiens
uucauuugguauaaaccgcgauu

SEQ ID NO: 171 - RNA, 23 nt, Homo sapiens
gaacgcgcuucccuauagagggu

SEQ ID NO: 172 - RNA, 22 nt, Homo sapiens
gaaaucaagcgugggugagacc

SEQ ID NO: 173 - RNA, 22 nt, Homo sapiens
ugagguaguagguuguauaguu

SEQ ID NO: 174 - RNA, 22 nt, Homo sapiens
ugagguaguagauuguauaguu

SEQ ID NO: 175 - RNA, 23 nt, Homo sapiens
agcagcauuguacagggcuauca

SEQ ID NO: 176 - RNA, 24 nt, Homo sapiens
ucccugagacccuuuaaccuguga

SEQ ID NO: 177 - RNA, 22 nt, Homo sapiens
accacugaccguugacuguacc

SEQ ID NO: 178 - RNA, 23 nt, Homo sapiens
aguuuugcagguuugcauccagc

SEQ ID NO: 179 - RNA, 23 nt, Homo sapiens
uaauacugccggguaaugaugga

SEQ ID NO: 180 - RNA, 22 nt, Homo sapiens
agagcuuagcugauuggugaac

SEQ ID NO: 181 - RNA, 21 nt, Homo sapiens
gccccugggccuauccuagaa

SEQ ID NO: 182 - RNA, 23 nt, Homo sapiens
ucccuguccuccaggagcucacg

SEQ ID NO: 183 - RNA, 24 nt, Homo sapiens
aauccuuggaaccuaggugugagu

SEQ ID NO: 184 - RNA, 22 nt, Homo sapiens
gccugcugggguggaaccuggu

SEQ ID NO: 185 - RNA, 22 nt, Homo sapiens
auauaauacaaccugcuaagug

SEQ ID NO: 186 - RNA, 21 nt, Homo sapiens
ugguagacuauggaacguagg

SEQ ID NO: 187 - RNA, 23 nt, Homo sapiens
uagugcaauauugcuuauagggu

SEQ ID NO: 188 - RNA, 22 nt, Homo sapiens
aaagugcuucccuuuggacugu

SEQ ID NO: 189 - RNA, 22 nt, Homo sapiens
ggagaaauuauccuuggugugu

SEQ ID NO: 190 - RNA, 22 nt, Homo sapiens
uuugugaccugguccacuaacc

SEQ ID NO: 191 - RNA, 22 nt, Homo sapiens
acuccagccccacagccucagc

SEQ ID NO: 192 - RNA, 23 nt, Homo sapiens
ucuuugguuaucuagcuguauga

SEQ ID NO: 193 - RNA, 22 nt, Homo sapiens
ugagguaguaaguuguauuguu

SEQ ID NO: 194 - RNA, 23 nt, Homo sapiens
ugucacucggcucggcccacuac

SEQ ID NO: 195 - RNA, 22 nt, Homo sapiens
aggcauugacuucucacuagcu

SEQ ID NO: 196 - RNA, 22 nt, Homo sapiens
ugguuuaccgucccacauacau

SEQ ID NO: 197 - RNA, 27 nt, Homo sapiens
cacuguaggugauggugagagugggca

SEQ ID NO: 198 - RNA, 20 nt, Homo sapiens
ugagcccuguccucccgcag

SEQ ID NO: 199 - RNA, 22 nt, Homo sapiens
acccgucccguucguccccgga

SEQ ID NO: 200 - RNA, 22 nt, Homo sapiens
acgcccuucccccccuucuuca

SEQ ID NO: 201 - RNA, 23 nt, Homo sapiens
cuggagauauggaagagcugugu

SEQ ID NO: 202 - RNA, 18 nt, Homo sapiens
gucccuguucaggcgcca

SEQ ID NO: 203 - RNA, 17 nt, Homo sapiens
gugggggagaggcuguc

SEQ ID NO: 204 - RNA, 22 nt, Homo sapiens
uucauucggcuguccagaugua

SEQ ID NO: 205 - RNA, 23 nt, Homo sapiens
uauggcuuuucauuccuauguga

SEQ ID NO: 206 - RNA, 23 nt, Homo sapiens
agcugguguugugaaucaggccg

SEQ ID NO: 207 - RNA, 23 nt, Homo sapiens
guccaguuuucccaggaaucccu

SEQ ID NO: 208 - RNA, 22 nt, Homo sapiens
uagcagcacauaaugguuugug

SEQ ID NO: 209 - RNA, 22 nt, Homo sapiens
aacauucaaccugucggugagu

SEQ ID NO: 210 - RNA, 22 nt, Homo sapiens
caaagaauucuccuuuugggcu

SEQ ID NO: 211 - RNA, 23 nt, Homo sapiens
acugcccuaagugcuccuucugg

SEQ ID NO: 212 - RNA, 22 nt, Homo sapiens
aacuggccuacaaagucccagu

SEQ ID NO: 213 - RNA, 23 nt, Homo sapiens
cccaguguuuagacuaucuguuc

SEQ ID NO: 214 - RNA, 22 nt, Homo sapiens
uaacacugucugguaacgaugu

SEQ ID NO: 215 - RNA, 22 nt, Homo sapiens
uccuucauuccaccggagucug

SEQ ID NO: 216 - RNA, 23 nt, Homo sapiens
caaagugcucauagugcagguag

SEQ ID NO: 217 - RNA, 22 nt, Homo sapiens
acagcaggcacagacaggcagu

SEQ ID NO: 218 - RNA, 22 nt, Homo sapiens
ugccugucuacacuugcugugc

SEQ ID NO: 219 - RNA, 21 nt, Homo sapiens
uugugcuugaucuaaccaugu

SEQ ID NO: 220 - RNA, 21 nt, Homo sapiens
ccaccaccgugucugacacuu

SEQ ID NO: 221 - RNA, 22 nt, Homo sapiens
ugucaguuugucaaauacccca

SEQ ID NO: 222 - RNA, 21 nt, Homo sapiens
aucacauugccagggauuacc

SEQ ID NO: 223 - RNA, 22 nt, Homo sapiens
cuuucagucggauguuugcagc

SEQ ID NO: 224 - RNA, 22 nt, Homo sapiens
cuuucagucggauguuuacagc

SEQ ID NO: 225 - RNA, 20 nt, Homo sapiens
ccucugggcccuuccuccag

SEQ ID NO: 226 - RNA, 23 nt, Homo sapiens
ugucugcccgcaugccugccucu

SEQ ID NO: 227 - RNA, 21 nt, Homo sapiens
ugucuugcaggccgucaugca

SEQ ID NO: 228 - RNA, 22 nt, Homo sapiens
uuuugcgauguguuccuaauau

SEQ ID NO: 229 - RNA, 22 nt, Homo sapiens
uuuugcaauauguuccugaaua

SEQ ID NO: 230 - RNA, 22 nt, Homo sapiens
uaugugccuuuggacuacaucg

SEQ ID NO: 231 - RNA, 22 nt, Homo sapiens
aaucauacagggacauccaguu

SEQ ID NO: 232 - RNA, 22 nt, Homo sapiens
ugaaggucuacugugugccagg

SEQ ID NO: 233 - RNA, 22 nt, Homo sapiens
ugaaacauacacgggaaaccuc

SEQ ID NO: 234 - RNA, 22 nt, Homo sapiens
aauccuuugucccugggugaga

SEQ ID NO: 235 - RNA, 22 nt, Homo sapiens
gggagccaggaaguauugaugu

SEQ ID NO: 236 - RNA, 21 nt, Homo sapiens
gugucuuuugcucugcaguca

SEQ ID NO: 237 - RNA, 21 nt, Homo sapiens
caaagcgcuucccuuuggagc

SEQ ID NO: 238 - RNA, 21 nt, Homo sapiens
aaagcgcuucccuucagagug

SEQ ID NO: 239 - RNA, 21 nt, Homo sapiens
gaaagcgcuucucuuuagagg

SEQ ID NO: 240 - RNA, 22 nt, Homo sapiens
caaaaaucucaauuacuuuugc

SEQ ID NO: 241 - RNA, 22 nt, Homo sapiens
cgaaaacagcaauuaccuuugc

SEQ ID NO: 242 - RNA, 21 nt, Homo sapiens
ugaguuggccaucugagugag

SEQ ID NO: 243 - RNA, 21 nt, Homo sapiens
uagauaaaauauugguaccug

SEQ ID NO: 244 - RNA, 23 nt, Homo sapiens
aaacucuacuuguccuucugagu

SEQ ID NO: 245 - RNA, 22 nt, Homo sapiens
cuguugccacuaaccucaaccu

SEQ ID NO: 246 - RNA, 22 nt, Homo sapiens
ugagaccucuggguucugagcu

SEQ ID NO: 247 - RNA, 22 nt, Homo sapiens
uccauuacacuacccugccucu

SEQ ID NO: 248 - RNA, 22 nt, Homo sapiens
cacuggcuccuuucuggguaga

SEQ ID NO: 249 - RNA, 22 nt, Homo sapiens
auaaagcuagauaaccgaaagu

SEQ ID NO: 250 - RNA, 22 nt, Homo sapiens
uucaacggguauuuauugagca